(19) United States
(12) Patent Application Publication
(10) Pub. No.: US 2025/0146919 A1
(43) Pub. Date: May 8, 2025

(54) SYSTEMS AND METHODS FOR DETECTING CONDITIONS IN AN ENVIRONMENT AROUND A DEVICE

(71) Applicant: N5 SENSORS, INC., Rockville, MD (US)

(72) Inventors: Brian THOMSON, Washington, DC (US); Vinicius Moraes FONSECA, Rockville, MD (US); Abhishek MOTAYED, Bethesda, MD (US)

(21) Appl. No.: 18/835,885

(22) PCT Filed: Feb. 7, 2022
(86) PCT No.: PCT/US2022/015437

§ 371 Date: Aug. 5, 2024

(51) Int. Cl. G01N 15/06; G01N 15/00; G01N 33/00

(52) U.S. Cl. CPC G01N 15/06; G01N 33/0037; G01N 33/0039; G01N 33/0042; G01N 2015/0046

(57) ABSTRACT

Systems and methods are provided for collecting and transmitting information associated with conditions in an environment around a device. The method includes: collecting, using at least one infrared (IR) camera, IR data of the environment around the device; collecting, using at least one gas sensor, gas concentration data of at least one gas; collecting, using at least one particulate matter detector, particulate matter concentrations in the environment around the device, wherein the at least one infrared camera, the at least one gas sensor and the at least one particulate matter detector forward their respective collected data to a radio; generating, using at least one solar panel, power for at least one of the at least one IR camera, the at least one gas sensor and the at least one particulate matter detector; and transmitting, using the radio, the collected data.

### TECHNICAL FIELD

The present invention relates to gas sensing devices and methods for reconfiguring gas sensing devices to detect different gases, i.e., relative to one or more gases for which a gas sensing device was previously configured to detect. The gas sensing devices can, for example, include optically-activated gas sensors, e.g., sensors which include a semiconductor nanostructure and at least one of metal or metal-oxide nanoparticles functionalizing the nanostructure and forming a hybrid sensor that enables light-assisted sensing of a target analyte. The present invention also relates to using gas sensing devices alone or in combination with other sensors to determine environmental conditions around the various devices in which the various sensors reside.

### BACKGROUND

Detection of chemical species in air, such as industrial pollutants, poisonous gases, chemical fumes, and volatile organic compounds (VOCs), is vital for the health and safety of communities around the world (see Watson J and Ihokura K (1999) Special issue on Gas-Sensing Materials, Mater. Res. Soc. Bull. 24:14). The development of reliable, portable gas sensors that can detect harmful gases in real-time with high sensitivity and selectivity is therefore extremely important (Wilson D M et al. (2001) “Chemical Sensors for Portable, Handheld Field Instruments,” IEEE Sensors Journal 1:256-274; Eranna G et al. (2004) “Oxide Materials for Development of Integrated Gas Sensors—A Comprehensive Review/Integrated Gas Sensors—A Comprehensive Review,” Critical Reviews in Solid State and Material Sciences 29:111-188).

Due to their small size, ease of deployment, and low-power operation, solid-state thin film sensors are favored over analytical techniques such as optical and mass spectroscopy, and gas chromatography for real-time environmental monitoring (Wilson D M et al. (2001), supra, IEEE Sensor Journal 1:256-274; Shimizu Y and Egashira M (1999) “Basic aspects and Challenges of Semiconductor Gas Sensors,” Mater. Res. Soc. Bull. 24:18; Sze S M (1994) Semiconductor Sensors 1^{st} ed, Willey; New York). Selectivity, which is a sensor's ability to discriminate between the components of a gas mixture and provide detection signal for the component of interest, is an important consideration for the sensor's real-life applicability. Conventional metal-oxide based thin film sensors, despite decades of research and development (Brattain J B W H (1952) “Surface properties of germanium,” Bell. Syst. Tech. Journal 32:1; Azad A M et al. (1992) “Solid-State Sensors: A Review,” J. Electrochem. Soc. 139 (12): 3690-3704), still lack selectivity for different species and typically require high working temperatures (Meixner H and Lampe U (1996) “Metal oxide sensors,” Sens. and Actuators B 33:198-202; Nicoletti S et al. (2003) “Use of Different Sensing Materials and Deposition Techniques for Thin-Film Sensors to Increase Sensitivity and Selectivity,” IEEE Sensors Journal 3:454-459; Demarne V and Sanjines R (1992) Gas Sensors-Principles, Operation and Developments ed. G. Sberveglieri, Kluwer Academic, Netherlands). As such, the usability of such conventional sensors is severely limited and poses long-term reliability problems.

For a chemical sensor, the active surface area is an important factor for determining its detection limits or sensitivity. It is known that the electrical properties of nanowires (NWs) change significantly in response to their environments due to their high surface to volume ratio (Cui Y et al. (2001), supra, Science 293:1289-1292; Zhang D et al. (2004) “Detection of NO₂ down to ppb levels using individual and multiple In₂O₃ nanowire devices,” Nano. Lett. 4:1919-1924; Kong J et al. (2000) “Nanotube Molecular Wires as Chemical Sensors,” Science 287:622-625; Comini E et al. (2002) “Stable and highly sensitive gas sensors based on semiconducting oxide nanobelts,” Appl. Phys. Lett. 81:1869). NWs are therefore well suited for direct measurement of changes in their electrical properties (e.g. conductance/resistance, impedance) when exposed to various analytes. Substantial research has demonstrated the enhanced sensitivity, reactivity, and catalytic efficiency of the nanoscale structures (Cui Y et al. (2001), supra, Science 293:1289; Li C et al. (2003) “In₂O₃ nanowires as chemical sensors,” Appl. Phys. Lett. 8:1613; Wan Q et al. (2004) “Fabrication and ethanol sensing characteristics of ZnO nanowire gas sensors,” Appl. Phys. Lett. 84:3654; Wang C et al. (2005) “Detection of H₂S down to ppb levels at room temperature using sensors based on ZnO nanorods,” Sens. and Actuators B 113:320-323; Wang H T et al. (2005) “Hydrogen-selective sensing at room temperature with ZnO nanorods,” Appl. Phys. Lett. 86:243503; Raible I et al. (2005) “V₂O₅ nanofibers: novel gas sensors with extremely high sensitivity and selectivity to amines,” Sens. and Actuators B 106:730-735; McAlpine M C et al. (2007) “Highly ordered nanowire arrays on plastic substrates for ultrasensitive flexible chemical sensors,” Nat Mater 6:379-384).

There have been attempts to demonstrate sensors based on nanotube/nanowire decorated with nanoparticles of metal and metal-oxides. For example, Leghrib et al. reported gas sensors based on multiwall carbon nanotubes (CNTs) decorated with tin-oxide (SnO₂) nanoclusters for detection of NO and CO (see Leghrib R et al. (2010) “Gas sensors based on multiwall carbon nanotubes decorated with tin oxide nanoclusters,” Sens. and Actuators B: Chemical 145:411-416). Using mixed SnO₂/TiO₂ included with CNTs, Duy et al. demonstrated ethanol sensing at a temperature of 250° C. (Duy N V et al. (2008) “Mixed SnO₂/TiO₂ Included with Carbon Nanotubes for Gas-Sensing Application,” J. Physica E 41:258-263). Balázsi et al. fabricated hybrid composites of hexagonal WO3 powder with metal decorated CNTs for sensing NO₂ (Balázsi C et al. (2008) “Novel hexagonal WO3 nanopowder with metal decorated carbon nanotubes as NO2 gas sensor,” Sensors and Actuators B: Chemical 133:151-155). Kuang et al. demonstrated an increase in the sensitivity of SnO₂ nanowire sensors to H₂S, CO, and CH4 by surface functionalization with ZnO or NiO nanoparticles (Kuang Q et al. (2008) “Enhancing the photon-and gas-sensing properties of a single SnO2 nanowire based nanodevice by nanoparticle surface functionalization,” J. Phys. Chem. C 112:11539-11544). ZnO NWs decorated with Pt nanoparticles were utilized by Zhang et al., showing that the response of Pt nanoparticles decorated ZnO NWs to ethanol is three times higher than that of bare ZnO NWs (Zhang Y et al. (2010) “Decoration of ZnO nanowires with Pt nanoparticles and their improved gas sensing and photocatalytic performance,” Nanotechnology 21:285501). Chang et al. showed that by adsorption of Au nanoparticles on ZnO NWs, the sensor sensitivity to CO gas could be enhanced significantly (Chang S-J et al. (2008) “Highly sensitive ZnO nanowire CO sensors with the adsorption of Au nanoparticles,” Nanotechnology 19:175502). Dobrokhotov et al. constructed a chemical sensor from mats of GaN NWs decorated with Au nanoparticles and tested their sensitivity to N₂ and CH₄ (Dobrokhotov V et al. (2006) “Principles and mechanisms of gas sensing by GaN nanowires functionalized with gold nanoparticles,” J. Appl. Phys 99:104302). GaN NWs coated with Pd nanoparticles were employed for the detection of H₂ in N₂ at 300K by Lim et al. (Lim W et al. (2008) “Room temperature hydrogen detection using Pd-coated GaN nanowires,” Appl. Phys. Lett. 93:072109).

Although such results demonstrate the potentials of the nanowire-nanocluster based hybrid sensors, fundamental challenges and deficiencies in such prior attempts remain. Most of the results provide for mats of nanowires. Although such mats may increase sensitivity, the complex nature of inter-wire conduction makes interpreting the results difficult. Also, room-temperature operation of such previous sensors has not been demonstrated, and the selectivity is shown for only a very limited number of chemicals. Conventional sensor devices require high operating temperatures (>250° C.) and large response times (more than 5 minutes). Indeed, such temperature-assisted sensors typically provide for an integrated heater for the device. Further, the reported sensitivities of such conventional devices were quite low even with long response times. Further, such conventional devices typically do not provide for air as the carrier gas. However, the ability of a sensor to detect chemicals in air is what ultimately determines its usability in real-life.

Thus, such demonstrations have resulted in poor selectivity of known chemical sensors, and therefore have not resulted in commercially viable gas sensors. For real-world applications, selectivity between different classes of compounds (such as between aromatic compounds and alcohols) is highly desirable. For example, the threat of terrorism and the need for homeland security call for advanced technologies to detect concealed explosives safely and efficiently. Detecting traces of explosives is challenging, however, because of the low vapor pressures of most explosives (Moore, D S (2004) “Instrumentation for trace detection of high explosives,” Review of Scientific Instruments 75 (8): 2499-2512; Yinon J (2002) “Field detection and monitoring of explosives,” TrAC Trends in Analytical Chemistry 21 (4): 292-301; Senesac L. and Thundat T G (2008) “Nanosensors for trace explosive detection,” Materials Today 11 (3): 28-36. Moreover, the difficulty of explosive detection is aggravated by the noisy environment which masks the signal from the explosive, the potential for high false alarms, and the need to determine a threat quickly. As such, trained canine teams remain the most reliable means of detecting explosive vapors to date; however, dogs are expensive to train and tire easily.

An ideal chemical sensor would be able to distinguish between the individual analytes belonging to a particular class of compounds, e.g. detection of the presence of benzene or toluene in the presence of other aromatic compounds, detection of a particular explosive compound, detection of a particular alcohol, etc. This is extremely challenging as most semiconductor-based sensors use metal-oxides (such as SnO₂, In₂O₃, ZnO) as the active elements, which are limited due to the non-selective nature of the surface adsorption sites. The surface/adsorbate interactions of conventional sensor structures are limited and non-specific. Thus, conventional sensor devices lack the same selectivity as their bulk-counterpart devices.

An ideal chemical sensor would be able to distinguish between the individual analytes belonging to a particular class of compounds, e.g., detection of the presence of benzene or toluene in the presence of other aromatic compounds, detection of a particular explosive compound, detection of a particular alcohol, etc. This is extremely challenging as most semiconductor-based sensors use metal-oxides (such as SnO.sub.2, In.sub.2O.sub.3, ZnO) as the active elements, which are limited due to the non-selective nature of the surface adsorption sites. The surface/adsorbate interactions of conventional sensor structures are limited and non-specific. Thus, conventional sensor devices lack the same selectivity as their bulk-counterpart devices.

U.S. Pat. No. 9,476,862, the disclosure of which is incorporated here by reference and which has one or more common inventors with the present application, describes nanostructure sensor devices that address these deficiencies of conventional devices by providing a semiconductor nanostructure having an outer surface and at least one of metal or metal-oxide nanoparticle clusters functionalizing the outer surface of the nanostructure and forming a photoconductive nanostructure/nanocluster hybrid sensor enabling light-assisted sensing of a target analyte. Additionally, U.S. patent application Ser. No. 15/891,709 entitled “Device Having an Array of Sensors on a Single Chip”, the disclosure of which is incorporated here by reference and which has one or more common inventors with the present application, describes using one or more nanoparticle gas sensors on a single chip, which sensors are configured to detect at least one type of gas.

Another area in which current conventional sensors are lacking is in the detection of chemicals associated with environmental conditions such as wildfires. Current sensing systems are believed to be expensive and include many of the drawbacks as described above with respect to room temperature sensors and analyte selectivity which would be a drawback in detecting conditions associated with wildfires or other types of environmental conditions. Further there is a lack of so-called “complete” systems which would start with the detection of an undesirable environmental condition and then proceed to use that detection to perform data transmission, processing, alert generation and display of alerts associated with the undesirable environmental condition.

The present application thus focuses on providing devices with sensors capable of obtaining information associated with environmental conditions which overcome the drawbacks of current sensing devices.

### SUMMARY

The present invention is directed to using one or more devices which are configured to determine environmental conditions, e.g., atmospheric conditions, associated with a plurality of use cases that generate measurable characteristics. For example, the use cases can include, but are not limited to, wildfires, air pollution monitoring and air monitoring for a variety of gases which can then be implemented in a variety of places, e.g., smart cities, airports, railways, offices, forests, other outdoor environments, and areas where monitoring the environment could be useful for public safety. To further include data transmission, processing, alert generation and display of such alerts associated with the determined environmental conditions.

According to an embodiment, there is a device configured to collect and transmit information associated with conditions in an environment around the device. The device comprising: at least one infrared (IR) camera configured to collect IR data of the environment around the device; at least one gas sensor configured to collect gas concentration data of at least one gas; at least one particulate matter detector configured to collect particulate matter concentrations in the environment around the device, wherein the at least one infrared camera, the at least one gas sensor and the at least one particulate matter detector forward their respective collected data to a radio; at least one solar panel configured to generate power for at least one of the at least one IR camera, the at least one gas sensor and the at least one particulate matter detector; and the radio configured to transmit the collected data.

According to an embodiment, there is a system for collecting data, processing the data, generating alerts and displaying the alerts in a real-time or near real-time basis associated with atmospheric content testing. The system comprising: at least one infrared (IR) camera configured to collect IR data of the environment around the device; at least one gas sensor configured to collect gas concentration data of at least one gas; at least one particulate matter detector configured to collect particulate matter concentrations in the environment around the device, wherein the at least one infrared camera, the at least one gas sensor and the at least one particulate matter detector forward their respective collected data to a radio, wherein the radio is configured to transmit the collected data to at least one server; at least one solar panel configured to generate power for at least one of the at least one IR camera, the at least one gas sensor and the at least one particulate matter detector; the at least one server configured to process the received data according to preconfigured instructions and to generate alerts based on a ruleset based on the processed data then to transmit the alerts; and a user interface configured to receive and display the alerts.

According to an embodiment, there is method for collecting and transmitting information associated with conditions in an environment around a device. The method comprising: collecting, using at least one infrared (IR) camera, IR data of the environment around the device; collecting, using at least one gas sensor, gas concentration data of at least one gas; collecting, using at least one particulate matter detector, particulate matter concentrations in the environment around the device, wherein the at least one infrared camera, the at least one gas sensor and the at least one particulate matter detector forward their respective collected data to a radio; generating, using at least one solar panel, power for at least one of the at least one IR camera, the at least one gas sensor and the at least one particulate matter detector; and transmitting, using the radio, the collected data.

### BRIEF DESCRIPTION OF THE DRAWINGS

The patent or application file contains at least one drawing/photograph executed in color. Copies of this patent or patent application publication with color drawing(s) will be provided by the Office upon request and payment of the necessary fee.

FIG. 1, plates (a) and (b), are schematic representations of a GaN (Nanowire)-TiO₂ (Nanocluster) hybrid sensor according to the present invention. Figure. 1, plate (a) shows the sensor in the dark showing surface depletion of the GaN nanowire, and FIG. 1, plate (b) shows the sensor under UV excitation with photodesorption of O₂ due to hole capture.

FIG. 2, plate (a), illustrates graphically the photoresponse of a hybrid device (diameter 300 nm) to 1000 ppm of benzene and toluene mixed in air and nitrogen. FIG. 2, plate (b) illustrates the response of a hybrid device (diameter 500 nm) to different concentrations of water in air.

FIG. 3 is a schematic representation of depletion in the TiO₂ NC in the presence of oxygen and water, and its effect on the photogenerated charge carrier separation in GaN NW. Circles in valence band indicate holes and circles in conduction band indicate electrons.

FIG. 4 illustrates graphically the photo-response of the GaN/(TiO₂—Pt) device to 1000 μmol/mol of ethanol in air and nitrogen, and to 1000 μmol/mol of water in air. The devices did not respond to water in nitrogen. The air-gas mixture was turned on at 0 s and turned off at 100 s.

FIG. 5, plate (a) illustrates graphically UV photo-response of the GaN/(TiO₂—Pt) hybrid device to 1000 μmol/mol (ppm) of methanol, ethanol, and water in air, and hydrogen in nitrogen. The air-gas mixture was turned on at 0 s and turned off at 100 s. FIG. 5, plate (b) illustrates the cyclic response of the GaN/(TiO₂—Pt) hybrid device when exposed to 2500 μmol/mol (ppm) of hydrogen in nitrogen. The bias voltage for all the devices was 5 V.

FIG. 6, plate (a) is a scanning electron microscope (SEM) image of the NW bridge structure according to the present invention. FIG. 6, plate (b) shows ZnO nanoparticles on the facets of GaN NW. FIG. 6, plate (c) illustrates graphically current-voltage (I-V) characteristics of the device before and after rapid thermal anneal (RTA). FIG. 6, plate (d) is an x-ray diffraction (XRD) Ω-2θ scan of a 300-nm-thick ZnO film.

FIG. 7 illustrates graphically device response to 500-μmol/mol (ppm) of methanol. The inset graph at the bottom left shows the sensitivity of two devices toward 500 μmol/mol (ppm) of each isomer of butanol (with Device 1 shown as the right bar above each isomer, and Device 2 shown as the left bar above each isomer). The inset graph at the bottom right shows the response to ethanol, acetone, benzene, and hexane. Sensitivity(S) is given by (I_{g}−Iₐ)×100/Iₐ, where I_{g} is the device current in the presence of an analyte in breathing air and Iₐ is the current in pure breathing air, both measured 300 s after the flow is turned on. Percentage standard deviation of the device sensitivity is 3.2% based on the five data points collected over a period of 3 days in response to the breathing air.

FIG. 8 illustrates graphically device response to different flow rates of breathing air (plate (a)) and nitrogen gas (plate (b)). The flow rates of the gas are denoted as α=20 sccm, b=40 sccm, c=60 sccm, d=80 sccm, and e=100 sccm.

FIG. 9, plate (a) is a schematic illustration of a nanostructured semiconductor-nanocluster hybrid gas sensor according to an embodiment of the present invention. The sensor works with low-intensity light from an LED. The emission wavelength is determined by the semiconductor and metal-oxide bandgaps. FIG. 9, plate (b) illustrates schematically an exemplary thin-film device including a semiconductor backbone functionalized with TiO₂ on a sapphire substrate. The smoothness of the substrate and film after thermal processing is shown in FIG. 9, plates (c) and (d).

FIG. 10 is a schematic illustration of the mechanism of sensing using the disclosed nanocluster-functionalized semiconductor devices. The sensing is due to the effective separation of photogenerated charge carriers in the semiconductor backbone caused by surface potential modification of the backbone by the nanocluster upon adsorption of chemicals. The light produces electron-hole pairs in the semiconductor, and also surface defects on the cluster due to photo desorption of oxygen and water.

FIG. 11 illustrates schematically the epitaxial layer structure utilized in sensor device fabrication according to an embodiment of the invention.

FIG. 12 illustrates schematically sensor designs according to the present invention, including a sensor having serial architecture (plate (a)), and a sensor having parallel architecture (plate (b)).

FIG. 13 are schematic illustrations of a series architecture design of a sensor with four segments, including a top view (plate (a)) and a cross-section view taken along the dashed line (plate (b)). The sensor output is the voltage between the +Vₛₑₙₛₒᵣ and ground pads. The V_{cal} are the real-time calibration probes for baseline and temperature drift compensating.

FIG. 14 illustrates graphically a generic sensor calibration curve. Sensitivity S is defined as the slope of the sensor output response vs. analyte concentration plot. The sensor output may be a change in current, voltage, or resistance.

FIG. 15 is a schematic illustration of photoexcitation of both the metal-oxide cluster and the GaN backbone using 365 nm light.

FIG. 16 is a schematic illustration showing selectivity tuning using a multicomponent design of nanoclusters. As shown, the target analyte is NO₂ and the interfering chemical is CO₂.

FIG. 17 illustrates graphically depletion depth induced by Pt nanoclusters on GaN and TiO₂ (as calculated by Equation (12) below).

FIG. 18 is a schematic illustration of an integration scheme for standalone system, showing components at roughly their actual size.

FIG. 19 is a schematic illustration of a hybrid sensor fabrication process according to the present invention.

FIG. 20, plates (a-c), are field-emission scanning electron microscopy (FESEM) images of three different sputtered thickness of TiO₂ coatings: including 2 nm (plate (a)), 5 nm (plate (b)), and 8 nm (plate (c)) of TiO₂ sputtered on GaN nanowires.

FIG. 21 illustrates graphically an XRD Ω-2θscan of 150 nm thick TiO₂ film deposited on SiO₂/Si substrate at 300° C. and annealed at 650° C. for 45 s in RTA. All indices correspond to the anatase phase [PDF #84-1285].

FIG. 22 illustrates typical morphologies of a 20 nm thick TiO₂ film sputtered on n-GaN nanowires and annealed at 700° C. for 30 s. FIG. 22, plate (a) is a TEM image showing non-uniformly distributed 2 nm to 10 nm diameter individual TiO₂ particles, with some of the particles marked by white circles. FIG. 22, plate (b) is a high-resolution transmission electron microscopy (HRTEM) image of an edge of the GaN nanowire with the sputtered TiO₂ film. The FFT pattern from the boxed area is shown in exploded view in the upper left inset, indicating 0.35 nm lattice fringes which are consistent with a (101) reflecting plane of anatase.

FIG. 23, plate (a) is a BF-STEM image with 5 nm to 10 nm TiO₂ nanoparticles barely visible near an edge of a GaN nanowire, with some of the nanoparticles marked by circles. FIG. 23, plate (b) is an ADF-STEM image of a TiO₂-containing aggregate on the edge of a GaN nanowire. FIG. 23, plate (c) is an X-ray spectrum of an individual 5 nm TiO₂ particle shown by circled portion ‘A’ in plate (a). FIG. 23, plate (d) is an EEL spectra recorded on position 1 (tip of the aggregate) and position 2 (edge of the GaN nanowire), as identified in plate (b), respectively.

FIG. 24 illustrates I-V characteristics of a GaN NW two-terminal device in the dark at different stages of processing. The inset shows the nanowire device with length 5.35 μm and diameter 380 nm. The scale bar is 4 μm. The thickness of sputtered TiO₂ film was 8 nm.

FIG. 25, plate (a) illustrates graphically the dynamic photocurrent of a bare GaN NW. FIG. 25, plate (b) illustrates the dynamic photocurrent of a TiO₂ coated (8 nm deposit) GaN NW. The diameters of both nanowires were about 200 nm. The applied bias is 5 V in both cases.

FIG. 26 illustrates graphically the dynamic response of a single GaN—TiO₂ hybrid device to 1000 ppm of toluene. For each cycle, the gas exposure time was 100 s. The inset shows the nanowire device with 8.0 μm length and diameter 500 nm. The scale bar is 5 μm.

FIG. 27, plate (a) illustrates the response of a single nanowire-nanocluster hybrid sensor (inset shows nanowire with diameter 500 nm) to 1000 ppm benzene, toluene, ethylbenzene, chlorobenzene, and xylene in presence of UV excitation. FIG. 27, plate (b) illustrates the response of a different sensor (inset shows nanowire with diameter 300 nm) to 200 ppb toluene, benzene, ethylbenzene, and xylene with UV excitation. The total flow in to the chamber was kept constant at 20 sccm. The response to air is also shown. The scale bars are 5 μm.

FIG. 28 illustrates graphically a hybrid sensor's photoresponse characteristics: FIG. 28, plate (a) shows the characteristics of the device shown in FIG. 27, plate (a) for 100 to 10000 ppm concentration range of toluene; FIG. 28, plate (b) shows the characteristics of the device shown in FIG. 27, plate (b) for 50 ppb to 1 ppm concentration range of toluene.

FIG. 29 illustrates sensitivity plots of a GaN—TiO₂ nanowire-nanocluster hybrid device (diameter 300 nm) for benzene, toluene, ethylbenzene, chlorobenzene, and xylene. The plot identifies the sensor's ability to measure wide range of concentration of the indicated chemicals.

FIG. 30 is an HRTEM image of a GaN NW with TiO₂ sputtered on them, with plate (a) showing the GaN NW before Pt and plate (b) showing after Pt deposition. Circled areas in plate (a) indicate partially aggregated polycrystalline TiO₂ particles on the NW surface and on the supporting carbon film. Arrows in plate (b) in the inset at the upper left mark Pt clusters decorating a 6 nm diameter particle of titanium. The TiO₂ particle exhibits 0.35 nm fringes corresponding to (101) lattice spacing of anatase polymorph. 2 nm to 5 nm thick amorphized surface film are indicated by black arrows.

FIG. 31 illustrate an HAADF-STEM of a GaN NW coated with TiO₂ and Pt., with plate (a) showing 1 nm to 5 nm bright Pt nanoparticles (shown by arrows) decorating surfaces of a polycrystalline TiO₂ island-like film and of a GaN nanowire. Medium grey aggregated TiO₂ particles (outlined by dashed line in plate (a)) are barely visible on a thin carbon support near the edge of the nanowire. Plate (b) is a high magnification image of the supporting film near the edge of the nanowire exhibiting 0.23 nm to 0.25 nm (111) and 0.20 nm to 0.22 nm (200) fcc lattice fringes belonging to Pt nanocrystallites, with arrows indicating amorphous-like Pt clusters of 1 nm and less in diameter.

FIG. 32 illustrates I-V characteristics of the hybrid sensor device at different stages of processing. FIG. 32, plate (a) shows GaN/(TiO₂—Pt) hybrids; FIG. 32, plate (b) shows GaN/Pt hybrids. The inset image in plate (b) shows the plan-view SEM image of a typical GaN NWNC hybrid sensor. The scale bar in the inset is 4 μm.

FIG. 33 illustrates graphically depletion depth induced by Pt NCs on GaN and TiO₂ as calculated by equation 12.

FIG. 34 illustrates comparative sensing behavior of the three hybrids for 1000 μmol/mol (ppm) of analyte in air: light gray bar graphs (benzene, toluene, ethylbenzene, xylene, chlorobenzene) represent GaN/TiO₂ hybrids, patterned bar graphs (ethanol, methanol, and hydrogen) represent GaN/(TiO₂—Pt) hybrids, and white bar graph (hydrogen) represents GaN/Pt hybrids. Other chemicals which did not produce any response in any one of the hybrids are not included in the plot. The zero line is the baseline response to 20 sccm of air and N₂. For this plot the magnitude of the sensitivity is used. The error bars represent the standard deviation of the mean sensitivity values for every chemical computed for 5 devices with diameters in the range of 200 nm-300 nm.

FIG. 35, plate (a) illustrates graphically the photo-response of GaN/(TiO₂—Pt) hybrid device to different concentrations of methanol in air. FIG. 35, plate (b) shows photo-response of the same device to different concentrations of hydrogen in nitrogen. The air-gas mixture was turned on at 0 s and turned off at 100 s.

FIG. 36, plate (a) is a sensitivity plot of the GaN/(TiO₂—Pt) hybrid device to ethanol, methanol, and water in air and to hydrogen in nitrogen ambient. FIG. 36, plate (b) shows graphically a comparison of the sensitivity of GaN/(TiO₂—Pt) and GaN/Pt devices to different concentrations of hydrogen in nitrogen.

FIG. 37 illustrates schematically an exemplary fabrication flow chart for semiconductor-nanocluster based gas sensors according to the present invention.

FIG. 38, plate (a) is an image of large area etched nanostructures of GaN on silicon and sapphire substrate formed according to disclosed processes such as shown in FIG. 37. FIG. 38, plate (b) shows an image of a nanostructure of GaN on silicon and sapphire using ICP etching and post-etching surface treatment. This nanostructure forms the backbone of the disclosed sensors in disclosed embodiments.

FIG. 39 is an RTEM image of a GaN NW with TiO₂ sputtered on them.

Circled portions indicate partially aggregated polycrystalline TiO₂ particles on the NW surface and on the supporting carbon film.

FIG. 40 illustrates graphically I-V characteristics of a GaN NW two-terminal device at different stages of processing.

FIG. 41, plate (a) illustrates graphically response of a single, nanowire-nanocluster hybrid sensor to 100 ppb of benzene, toluene, nitrobenzene, nitrotoluene, dinitrobenzene, dinitrotoluene and trinitrotoluene in the presence of UV excitation. FIG. 41, plate (b) shows the response of the device to different concentrations of trinitrotoluene.

FIG. 42 is a sensitivity plot of a GaN—TiO₂ nanowire-nanocluster hybrid device for benzene, toluene, nitrotoluene, nitrobenzene, DNT, DNB and TNT.

FIG. 43 illustrates sensitivity of two different nanowire-nanocluster hybrid sensors to 100 ppb of the different aromatic compounds.

FIG. 44, plate (a), illustrates the dynamic responses of a TiO₂ based sensor exposed to 250 ppm NO₂ mixed with breathing air under UV illumination and dark at room temperature. Plate (b) illustrates the response under UV at mixture of 100 ppm, 250 ppm, and 500 ppm with breathing air. The inset in plate (b) shows the measured responses under UV as a function of NO₂ concentrations with uncertainty. Sensitivity S is presented by (I_{g}−Iₐ)×100/Iₐ, wherein I_{g} is the device current in the presence of an analyte in breathing air and Iₐ is the current in pure breathing air, both measured 300 s after the flow is turned on.

FIG. 45 illustrates schematically an NO₂ gas sensing mechanism of the TiO₂ sensor under UV illumination: plate (a) shows the mechanism in a dark environment with breathing air in; plate (b) shows the mechanism under UV illumination in breathing air; and plate (c) shows the mechanism under UV illumination with mixture of NO₂ and breathing air (all at room temperature).

FIG. 46 illustrates graphically the dynamic response of the TiO₂ based sensor exposed to 500 ppm NO₂ under UV illumination and under dark at room temperature.

FIG. 47, plate (a) illustrates a GIXRD scan of thermally processed ultrathin TiO₂ film, and plate (b) illustrates optical properties (bandgap).

FIG. 48, plate (a) illustrates schematically a SnO₂—Cu nanocluster CO₂ sensor. Plates (b) and (c) are AFM images of the SnO₂—Cu nanocluster CO₂ sensor.

FIG. 49 illustrates the dynamic responses of the SnO₂—Cu based sensor exposed to CO₂ at room temperature at concentrations of 1000 ppm and 5000 ppm. For each cycle, the gas exposure time was 300 s.

FIG. 50 illustrates graphically the response of the SnO₂ based sensor at different relative humidity (RH) concentrations at room temperature.

FIG. 51, plate (a) illustrates the dynamic response of a TiO₂ based sensor exposed to methanol at room temperature and at a concentration of 500 ppm. Plate (b) illustrates the dynamic response of a ZnO based sensor exposed to benzene at room temperature and a concentration of 500 ppm. Plate (c) illustrates the dynamic response of the ZnO based sensor exposed to hexane at room temperature and a concentration of 100 ppm.

FIG. 52 illustrates graphically the dynamic responses of a ZnO—Pd—Ag based sensor exposed to H₂ at room temperature.

FIG. 53 illustrates schematically an exemplary layout of on chip elements of a sensor device in accordance with the present invention.

FIG. 54 illustrates schematically a micro-heater embedded into a sensor device in accordance with disclosed embodiments of the present invention.

FIG. 55 illustrates the temperature profile of 50 μm microheater made from a Ti/Ni metal stack MH recorded at 5 V bias voltage (plate (a)) and 10 V bias voltage (plate (b)).

FIG. 56 illustrates graphically the dynamic response of a functionalized GaN sensor device for sensing H₂S (concentration 50 ppm) in dry air.

FIG. 57 illustrates graphically the dynamic response of a functionalized GaN sensor device for sensing NO₂ (concentration 500 ppm) in dry air (22° C., relative humidity 0-5%).

FIG. 58 illustrates graphically the dynamic response of a functionalized GaN sensor device for sensing SO₂ (concentration 10 ppm) in dry air (22° C., relative humidity 0-5%).

FIG. 59 illustrates graphically the dynamic response of a functionalized GaN sensor device for sensing CO₂ (concentration 5000 ppm) in dry air (22° C., relative humidity 0-5%).

FIG. 60 illustrates an exemplary sensor module in accordance with the present invention.

FIG. 61 shows a gas sensor system;

FIG. 62 shows a nanowire photoconductive chemiresistor;

FIG. 63 illustrates a sensor according to an embodiment;

FIG. 64 shows a multi-gas sensor according to an embodiment;

FIG. 65 a flowchart of a process which results in a single package gas sensor according to an embodiment;

FIGS. 66A and 66B show a top and bottom portion of a single gas sensor package prior to assembly completion according to an embodiment;

FIG. 67 shows a single package gas sensor according to an embodiment;

FIG. 68 shows a sketch of a portion of the single package gas sensor according to an embodiment;

FIG. 69 shows another sketch of a portion of the single package gas sensor according to an embodiment;

FIG. 70 shows another sketch of a portion of the single package gas sensor according to an embodiment;

FIG. 71 shows an example of a desired placement of an LED die and gas sensor die on a sensor die and a representation of a gas ingress location according to an embodiment;

FIG. 72 shows another example of a desired placement of the LED die and

gas sensor die on the sensor die and the representation of the gas ingress location according to an embodiment;

FIG. 73 show an example of an undesired placement of an LED die and gas sensor die on a sensor die and a representation of a gas ingress location;

FIG. 74 shows another example of an undesired placement of the LED die and gas sensor die on the sensor die and the representation of the gas ingress location;

FIG. 75 shows a flowchart of a method for assembling a single package gas sensor according to an embodiment;

FIG. 76 is a block diagram of an ASIC controller for controlling gas sensors according to an embodiment;

FIG. 77 is a block diagram of a portion of the ASIC controller of FIG. 76 shown in more detail;

FIG. 78 is another block diagram of a portion of the ASIC controller of FIG. 76 shown in more detail;

FIG. 79 is a flow chart illustrating a method according to an embodiment;

FIG. 80 shows a neural network architecture according to an embodiment;

FIG. 81 is a graph representing the performance of the backpropagation artificial neural network (BPANN) according to an embodiment;

FIG. 82 shows a sequence for detecting Cl₂ according to an embodiment;

FIG. 83 shows a confusion matrix according to an embodiment;

FIG. 84 depicts another neural network architecture according to an embodiment;

FIG. 85 shows a graph representing the performance of another BPANN according to an embodiment;

FIG. 86 illustrates a sequence for detecting H₂S according to an embodiment;

FIG. 87 shows another confusion matrix according to an embodiment;

FIG. 88 is a flow chart illustrating a method for reconfiguring a gas sensing device according to an embodiment;

FIG. 89 shows a communications interface communicating with a gas sensing device to reconfigure same according to an embodiment;

FIG. 90 shows a device for detecting conditions in an environment according to an embodiment;

FIG. 91 shows a sensor package in the device according to an embodiment;

FIG. 92 depicts a radio according to an embodiment;

FIG. 93 illustrates a system for supporting a process of detecting environmental conditions in an environment;

FIG. 94 shows the steps performed when using one or more devices and

the system during a controlled burn test according to an embodiment;

FIG. 95 shows an alert map associated with the controlled burn test according to an embodiment;

FIG. 96 shows a timing chart associated with the controlled burn test according to an embodiment;

FIG. 97 depicts a summary of alerts associated with the controlled burn test according to an embodiment; and

FIG. 98 shows a method for collecting and transmitting information associated with conditions around a device according to an embodiment.

### DETAILED DESCRIPTION

The present invention is directed to controllers for sensor devices, e.g., sensors including a semiconductor nanostructure, such as a micro or nanodevice, or nanowire (NW), having a surface functionalized or decorated with metal or metal-oxide nanoparticles or nanoclusters. When metal/metal-oxide nanoparticles selected according to the disclosed methods are placed on the surface of a nanostructure, significant changes result in the physical properties of the system. The nanoparticles increase the adsorption of chemical species by introducing additional adsorption sites, thereby increasing the sensitivity of the resulting system. Further embodiments describe single package gas sensors and controllers for such sensors. Before discussing single package gas sensors and controllers (see headers below), a detailed discussion of exemplary sensors which can be packaged and/or controlled in accordance with those embodiments will first be discussed (reproduced, at least in large part, from the above cited '862 patent).

The metal or metal-oxide nanoparticles may be selected to act as catalysts designed to lower the activation energy of a specific reaction, which produces active radicals by dissociating the adsorbed species. These radicals can then spill-over to a semiconductor structure (see Sermon P A and Bond G C (1973) “Hydrogen Spillover,” Catal. Rev. 8 (2): 211-239; Conner W C et al. (1986) “Spillover of sorbed species,” Adv. Catal. 34:1), where they are more effective in charge carrier transfer. Further, the selected nanoparticles modulate the current through the nanowire through formation of nanosized depletion regions, which is in turn a function of the adsorption on the nanoparticles. Nanoparticles or nanoclusters suitable for the present invention include virtually any metal-oxide and/or metal. Thus, it should be understood that the present invention is not limited to the particular exemplary metal-oxides and/or metals disclosed in the various embodiments and examples herein.

According to one embodiment, nanowire-nanocluster hybrid chemical sensors were realized by functionalizing n-type (Si doped) gallium nitride (GaN) NWs with TiO₂ nanoclusters. The sensors selectively sense benzene and related aromatic environmental pollutants, such as toluene, ethylbenzene, and xylene (sometimes referred to as BTEX). GaN is a wide-bandgap semiconductor (3.4 eV), with unique properties (Morkoç H (1999) Nitride Semiconductors and Devices, Springer series in Materials Science, Vol. 32, Springer, Berlin). Its chemical inertness and capability of operating in extreme environments (high-temperatures, presence of radiation, extreme pH levels) is thus suitable for the disclosed sensor design. TiO₂ is a photocatalytic semiconductor with a bandgap energy of 3.2 eV (anatase phase). Photocatalytic oxidation of various organic contaminants over titanium dioxide (TiO₂) has been previously studied (see Mills A and Hunte S L (1997) “An overview of of semiconductor photocatalysis,” J. Photochem. Photobiol. A 108:1-35; Luo Y and Ollis D F (1996) “Heterogeneous photocatalytic oxidation of trichloroethylene and toluene mixtures in air: Kinetic promotion and inhibition, time-dependent catalyst activity,” J. Catal. 163:1-11). The TiO₂ nanoclusters were thus selected to act as nanocatalysts to increase the sensitivity, lower the detection time, and enable the selectivity of the structures to be tailored to organic analytes.

The hybrid sensor devices may be developed by fabricating two-terminal devices using individual GaN NWs followed by the deposition of TiO₂ nanoclusters using radio frequency (RF) magnetron sputtering. The sensor fabrication process employed standard micro-fabrication techniques. X-ray diffraction (XRD) and high-resolution analytical transmission electron microscopy using energy-dispersive X-ray and electron energy-loss spectroscopies confirmed the presence of anatase phase in TiO₂ clusters after post-deposition anneal at 700° C.

A change of current was observed for these hybrid sensors when exposed to the vapors of aromatic compounds (e.g., benzene, toluene, ethylbenzene, xylene, and chlorobenzene mixed with air) under UV excitation, while they had minimal or no response to non-aromatic organic compounds such as methanol, ethanol, isopropanol, chloroform, acetone, and 1, 3-hexadiene. The sensitivity range for the noted aromatic compounds, except chlorobenzene, were from about 1% down to about 50 parts per billion (ppb) at room-temperature. By combining the enhanced catalytic properties of the TiO₂ nanoclusters with the sensitive transduction capability of the nanowires, an ultra-sensitive and selective chemical sensing architecture is achieved.

As discussed in further detail in Example 1 below, GaN—TiO₂ (nanowire-nanocluster) hybrid sensors demonstrated a response to specific volatile organic compounds mixed with air at ambient temperature and humidity. In the presence of UV light (e.g., having a wavelength in the range of about 10 nm to about 400 nm), these hybrid sensor devices exhibited change in the photocurrent when exposed to benzene, toluene, ethylbenzene, xylene, and chlorobenzene mixed in air. However, gases like methanol, ethanol, isopropanol, chloroform, acetone, and 1, 3-hexadiene exhibited little or no change in the electrical characteristics of the devices, thus demonstrating the selective response of these sensors to the aromatic compounds. Benzene, toluene, ethylbenzene, and xylene were detected by the disclosed sensors at a concentration level as low as 50 ppb in air. In addition, the disclosed sensor devices are highly stable and able to sense aromatic compounds in air reliably for a wide range of concentrations (e.g., 50 ppb to 1%).

In addition, the disclosed sensors demonstrated highly sensitive and selective detection of traces of nitro-aromatic explosive compounds. As discussed in further detail in Example 5 below, GaN/TiO₂ nanowire-nanocluster hybrid sensors detected different aromatic and nitroaromatic compounds at room temperature. For example, the GaN/TiO₂ hybrids were able to detect trinitrotoluene (TNT) concentrations as low as 500 μmol/mol (ppt) in air and dinitrobenzene concentrations as low as 10 nmol/mol (ppb) in air in approximately 30 seconds. The noted sensitivity range of the devices for TNT was from 8 ppm down to as low as 500 ppt. The detection limit of dinitrotoluene, nitrobenzene, nitrotoluene, toluene and benzene in air is about 100 ppb with a response time of =75 seconds. Devices according to the present invention exhibited sensitive and selective response to TNT when compared to interfering compounds like toluene. Thus, the disclosed sensors are suitable for use as highly sensitive, selective, low-power and smart explosive detectors, which are relatively inexpensive to manufacture in larger quantities.

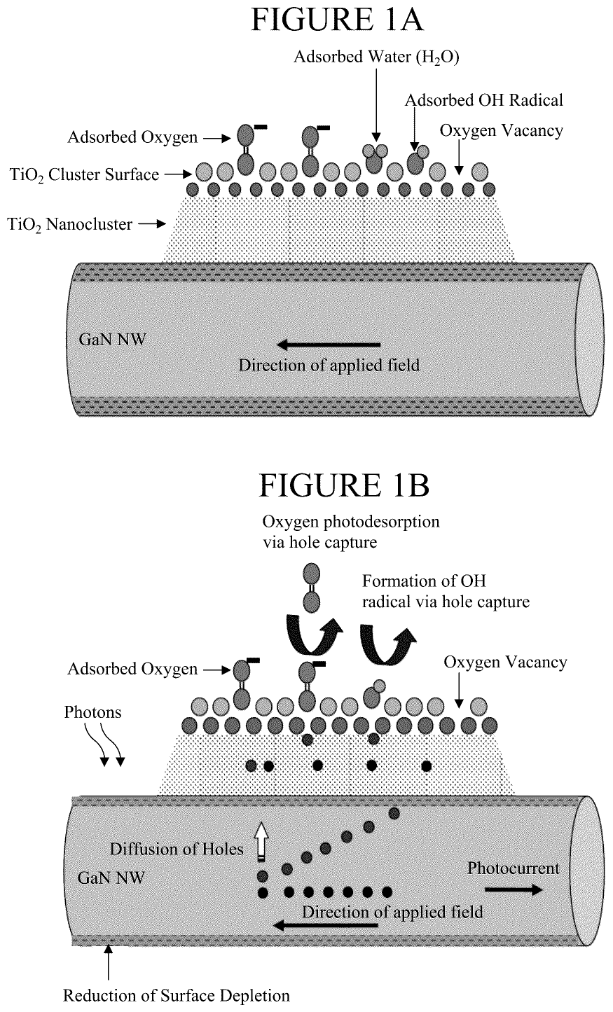

Based on structural analysis, an exemplary mechanism that qualitatively explains the hybrid sensor's response to different analytes is shown in FIG. 1. With regard to the photocatalytic processes on the TiO₂ surface, the oxygen vacancy defects (Ti³⁺ sites) on the surface of TiO₂ are the active sites responsible for adsorption of species like oxygen, water, and organic molecules (see Yates Jr J T (2009) “Photochemistry on TiO2: mechanisms behind the surface chemistry,” Surf. Sci. 603:1605-1612). Interestingly, a relatively defect free TiO₂ surface, generated by annealing in high-oxygen flux, is chemically inactive (Li M et al. (1999) “Oxygen-induced restructuring of rutile TiO₂ (110): formation mechanism, atomic models, and influence on surface chemistry,” Faraday Discuss. 114:245). Experimental studies and simulations reveal that molecular oxygen is chemisorbed on the surface vacancies (Ti³⁺ sites), acquiring a negative charge as shown in FIG. 1, plate (a) (Anpo M et al. (1999) “Generation of superoxide ions at oxide surfaces,” Top. Catal. 8:189-198; de Lara-Castells M P and Krause J L (2003) “Theoretical study of the UV-induced desorption of molecular oxygen from the reduced TiO₂ (110) surface,” J. Chem. Phys. 118:5098). This is due to the presence of the localized electron density at or near exposed Ti³⁺ atoms on the TiO₂ surface (Henderson M A et al. (1999) “Interaction of Molecular Oxygen with the Vacuum-Annealed TiO2(110) Surface: Molecular and Dissociative Channels,” J. Phys. Chem. B 103:5328-5337). Water may also be present on the TiO₂ cluster surface via molecular or dissociative adsorption, producing OH species on the defect sites (Lee F K et al. (2007) “Role of water adsorption in photoinduced superhydrophilicity on TiO₂ thin films,” Appl. Phys. Lett. 90:181928; Bikondoa O et al. (2006) “Direct visualization of defect-mediated dissociation of water on TiO₂ (110),” Nat. Mater. 5:189-192).

Although most of the theoretical and experimental studies on oxygen and water adsorption are done for the (110) surface of rutile phase, there are studies that suggest that similar adsorption behavior is also expected for the anatase surface (Wahab H S et al. (2008) “Computational investigation of water and oxygen adsorption on the anatase TiO₂ (100) surface,” J. Mol. Chem. Struct. 868:101-108). The GaN NW has a surface depletion region as shown in FIG. 1, plate (a), which determines its dark conductivity (Sanford N A et al. (2010) “Steady-state and transient photoconductivity in c-axis GaN nanowires grown by nitrogen-plasma-assisted molecular beam epitaxy,” J. Appl. Phy. 107:034318).

In the presence of UV excitation with an energy above the bandgap energy of anatase TiO₂ (3.2 eV) and GaN (3.4 eV), electron-hole pairs are generated both in the GaN NW and in the TiO₂ cluster, as shown in FIG. 1, plate (b). Photogenerated holes in the nanowire tend to diffuse towards the surface due to the surface band bending. This effect of separation of photogenerated charge carriers results in a longer lifetime of photogenerated electrons, which in turn enhances the photoresponse of the nanowire devices. On the TiO₂ cluster surface, however, the photogenerated charge carriers lead to a different phenomenon. In n-type semiconductor oxides such as TiO₂, the surface adsorption produces upward band-bending, which drives the photogenerated holes towards the surface. The chemisorbed oxygen molecule (O₂) and hydroxide ions (OH) can readily capture a hole and desorb as shown in FIG. 1, plate (b) (Perkins C L and Henderson M A (2001) “Photodesorption and Trapping of Molecular Oxygen at the TiO₂(110)—Water Ice Interface,” J. Phys. Chem. B. 105:3856-3863; Thompson T L and Yates J T Jr. (2006) “Control of a surface photochemical process by fractal electron transport across the surface: O(2) photodesorption from TiO(2)(110),” J. Phys. Chem. B 110:7431-7435). The decrease of photocurrent through these hybrid sensors when exposed to 20 sscm of air may be due to the increase in oxygen concentration at the surface of TiO₂ clusters, leading to an increase in trapping of photogenerated holes at the surface. This process results in increased lifetime of photogenerated electrons. As these nanowires are n-type, excess negative charge on the surface of the wire (on the TiO₂ clusters) reduces the nanowire current, thus providing a local-gating effect due to net negative charge accumulation in the TiO₂ clusters. Thus, the photoinduced oxygen desorption and subsequent capture of holes by organic adsorbate molecules on the surface of TiO₂ clusters produces the local-gating effect, which is responsible for the sensing action of the disclosed sensor devices. The adsorbed hydroxyl ions may also trap a hole forming OH species. Other effects such as diffusion of carriers between the clusters and the nanowire may also have a role in the sensing properties of the sensors.

Although some embodiments are described in term of excitation in the presence of UV light, it should be understood that excitation by radiation of other wavelengths may be more suitable for devices having other types of metal-oxide and/or metal nanoparticles. For example, excitation in the presence of visible light (i.e., having a wavelength of between about 380 nm and about 740 nm) is suitable for some embodiments.

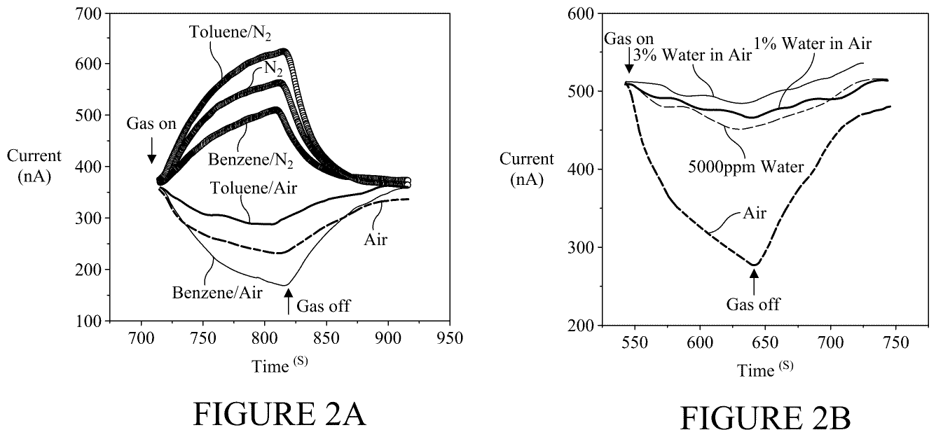

The process noted above and shown in FIG. 1 also explains sensor response when exposed to N₂ flow, as shown in FIG. 2, plate (a). In the presence of 20 sccm of N₂ flow, the photocurrent in the sensors increases significantly in comparison with 20 sccm of air flow. In an N₂ environment, oxygen is desorbed from the surface vacancy sites by capturing photogenerated holes, but does not get re-adsorbed, resulting in significant reduction of hole capture. As such, the photogenerated electron-hole pairs recombine effectively in the cluster. Thus, the photocurrent through the nanowire/nanocluster hybrid sensor, which is otherwise increased due to the local-gating effect by the TiO₂ clusters, is absent in an N₂ environment.

In the presence of water in air, the photocurrent through these sensors recovers towards the level without air flow, as seen in FIG. 2, plate (b), indicating a reduction of the hole trapping due to adsorption of water on the TiO₂ surface. Water may be adsorbed as a molecule on the defect sites replacing O₂ (see Herman G S et al. (2003) “Experimental Investigation of the Interaction of Water and Methanol with Anatase-TiO₂(101),” J. Phys. Chem. B 107:2788-2795). With increasing water concentration, more defects are filled with water. If the adsorbed water dissociates and produces OH species, then it is possible that it will act as hole traps and decrease the photocurrent the same way the photodesorption of oxygen does. A competition between the molecular water adsorption (reducing hole capture) and dissociative water adsorption (increasing hole capture) is possible, with the dominant process ultimately determining the photocurrent level in the nanowires in the presence of water.

The presence of aromatic compounds such as benzene, ethylbenzene, chlorobenzene, and xylene in air reduced the photocurrent (e.g. see FIG. 2, plate (a)). Organic molecules are known hole-trapping adsorbates (see Yamakata A et al. (2002) “Electron-and hole-capture reactions on Pt/TiO₂ photocatalyst exposed to methanol vapor studied with time-resolved infrared absorption spectroscopy,” J. Phys. Chem. B 106:9122-9125). Most aromatic compounds show high affinity for electrophilic aromatic substitution. The exact mechanism of photooxidation of adsorbed organic compounds on TiO₂ is complex. However, it is believed that oxidation occurs by either indirect oxidation via the surface-bound hydroxyl radical (i.e., a trapped hole at the TiO₂ surface) or directly via the valence-band hole before it is trapped either within the particle or at the particle surface (see Nosaka Y et al. (1998) “Factors governing the initial process of TiO2 photocatalysis studied by means of in situ electron spin resonance measurements,” J. Phys. Chem. B 102:10279-10283; Mao Y et al. (1991) “Identification of organic acids and other intermediates in oxidative degradation of chlorinated ethanes on titania surfaces en route to mineralization: a combined photocatalytic and radiation chemical study,” J. Phys. Chem. 95:10080-10089). In the presence of air (with residual water) hydroxyl mediated hole transfer to adsorbates such as benzene, xylene is dominant, whereas in the N₂ environment direct transfer of valence band holes to aromatic adsorbates could be possible.

Irrespective of the hole transfer mechanism, the presence of additional hole traps reduces the sensor photocurrent, as observed in the presence of benzene mixed with N₂ and air as shown in FIG. 2, plate (a). The model disclosed herein qualitatively explains the observed trends for compounds tested, such as benzene, ethylbenzene, chlorobenzene, and xylene. However, toluene exhibits a different trend, which may be due to other second order effects other than or in addition to the hole trapping mechanism.

The disclosed mechanism is further validated when comparing ionization energies of various compounds tested with the responses generated when the sensors are exposed to them (see Table I). The effectiveness of the process of hole transfer to the adsorbed organic molecules relates to the compound's ability to donate an electron (i.e. the lower the ionization energy of a compound, the easier for it to donate an electron or capture a hole). The observed sensitivity trend for benzene (lowest sensitivity), ethylbenzene, and xylene (highest sensitivity) correlates with their ionization energies as shown in Table I, with benzene being the highest and xylene the lowest among the three.

TABLE I
Physical Properties of Various Compounds Tested

| Organic Compound | Sensitivity | Ionization Potential (eV) |
|---|---|---|
| Chloroform | No | 11.37 |
| Ethanol | No | 10.62 |
| Isopropanol | No | 10.16 |
| Cyclohexane | Yes | 9.98 |
| Acetone | No | 9.69 |
| Benzene | Yes (Min) | 9.25 |
| Chlorobenzene | Yes | 9.07 |
| Toluene | Yes | 8.82 |
| Ethylbenzene | Yes | 8.77 |
| Xylene | Yes (Max) | 8.52 |
| 1,3-Hexadiene | No | 8.50 |

As shown in Table I, the sensitivity trend is consistent for aromatics, given 1,3-Hexadiene produced no response in the sensors. Although most functional groups with either a non-bonded lone pair or p-conjugation show oxidative reactivity towards TiO₂ (Hoffman M R et al. (1995) “Environmental Applications of Semiconductor Photocatalysis,” Chem. Rev. 95:69-96), aromatic compounds are more easily photocatalyzed than aliphatic ones under the same conditions (Carp O et al. (2004) “Photoinduced reactivity of titanium dioxide,” Prog. Solid St. Chem. 32:33-177).

Thus, the metal-oxide nanoclusters (TiO₂) on GaN NWs or nanostructures demonstrate the disclosed architecture for highly selective gas sensing. The exemplary sensors are capable of selectively sensing benzene and related aromatic compounds at nmol/mol (ppb) level in air at room-temperature under UV excitation.

According to another embodiment, the specific selectivity of the disclosed nanowire (or nanostructure)/nanocluster hybrid sensors may be tailored using a multi-component nanocluster design. For example, catalytic metals (e.g., platinum (Pt), palladium (Pd), and/or any other transition metals) are deposited onto the surface of oxide photocatalysts in order to enhance their catalytic activity. Metal clusters on a metal-oxide catalyst alter the behavior of the metal-oxide catalyst by any one, or a combination of, the following mechanisms: 1) changing the surface adsorption behavior as metals often have very different heat of adsorption values compared to the metal-oxides; 2) enabling catalytic decomposition of certain analytes on the metal surface, which otherwise would not be possible on the oxide surface; 3) transporting active species to the metal-oxide support by the spill-over effect from the metal cluster; 4) generating a higher degree of interface states, thus increasing reactive surface area reaction area; 5) changing the local electron properties of the metal clusters, such as workfunction, due to adsorption of gases; and 6) effectively separating photogenerated carriers in the underlying metal-oxide. The effect of transition metal loading such as iron (Fe), copper (Cu), Pt, Pd, and rhodium (Rh) onto TiO₂ has been evaluated for photocatalytic decomposition of various chemicals in both gas-solid and liquid-solid regimes.

In one implementation, the selectivity of the titanium dioxide (TiO₂) nanocluster-coated gallium nitride (GaN) nanostructure sensor device is altered by addition of platinum (Pt) nanoclusters. In another implementation, the sensor device includes Pt nanocluster-coated GaN nanostructure. The hybrid sensor devices may be developed by fabricating two-terminal devices using individual GaN NWs or nanostructures followed by the deposition of TiO₂ and/or Pt nanoclusters (NCs) using a sputtering technique, as described above.

The sensing characteristics of GaN/(TiO₂—Pt) nanowire-nanocluster (NWNC) hybrids and GaN/(Pt) NWNC hybrids is altered as compared to GaN/TiO₂ sensors. The GaN/TiO₂ NWNC hybrids show remarkable selectivity to benzene and related aromatic compounds with no measurable response for other analytes, as discussed above. However, the addition of Pt NCs to GaN/TiO₂ sensors dramatically alters the sensing behavior, making them sensitive only to methanol, ethanol, and hydrogen, but not to other chemicals tested, as discussed in further detail in Example 2 below.

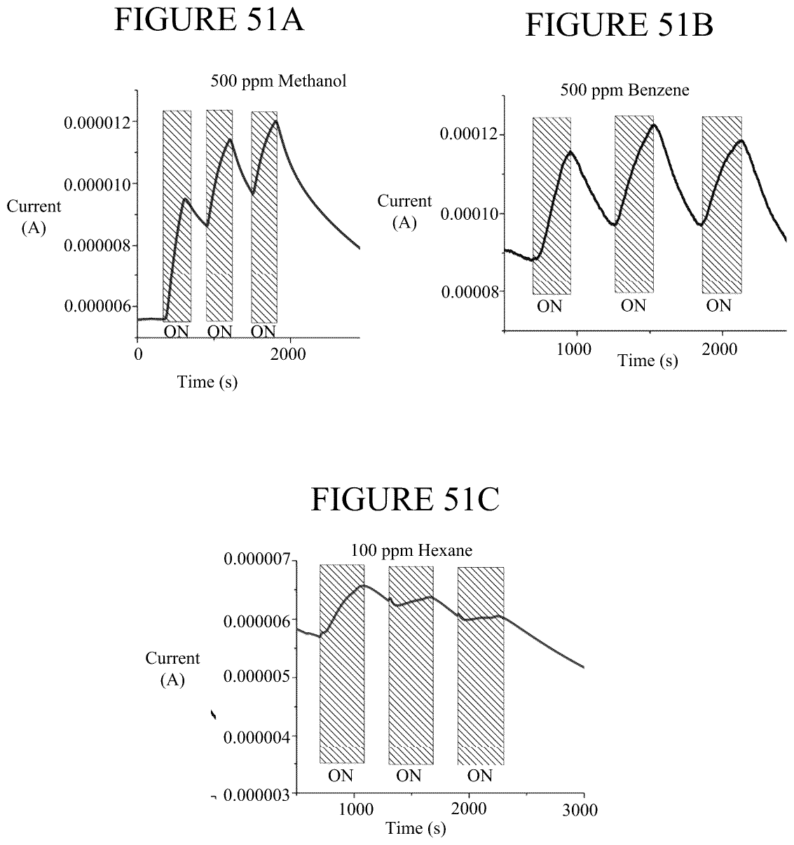

The GaN/(TiO₂—Pt) hybrid sensors were able to detect ethanol and methanol concentrations of 100 nmol/mol (ppb) in air in approximately 100 seconds, and hydrogen concentrations from 1 μmol/mol (ppm) to 1% in nitrogen in less than 60 seconds. However, GaN/Pt hybrid sensors showed limited sensitivity only towards hydrogen and not towards any alcohols. All the hybrid sensors are operable at room temperature and are photomodulated (i.e., responding to analytes only in the presence of light, e.g., ultra violet (UV) light). The selectivity achieved is significant from the standpoint of numerous applications requiring room-temperature sensing, such as hydrogen sensing and sensitive alcohol monitoring. For example, the dynamic response of an exemplary TiO₂ based sensor exposed to methanol at room temperature and at a concentration of 500 ppm is illustrated in FIG. 51, plate (a). The disclosed sensors therefore demonstrate tremendous potential for tailoring the selectivity of the hybrid nanosensors for a multitude of environmental and industrial sensing applications.

A qualitative understanding of the selective sensing mechanism of the disclosed sensors may be developed by considering how different molecules adsorb on the nanocluster surfaces, and determining the roles of intermediate reactions in the sensitivity of the sensors. While some of the embodiments, examples and explanation describe the invention in terms of NWs, it should be understood that other nanostructures or microstructures may be utilized. Accordingly, the present invention is not limited to sensors including NWs.

### The Photocurrent in GaN/(TiO₂—Pt) Hybrid Sensors in the Presence of Air, Nitrogen, and Water:

The oxygen vacancy defects (Ti³⁺ sites) on the surface of TiO₂ are the “active sites” for the adsorption of species like oxygen, water, and organic molecules (Yates Jr J T (2009) “Photochemistry on TiO2: mechanisms behind the surface chemistry,” Surf. Sci. 603:1605-1612; Bikondoa O et al. (2006) “Direct visualization of defect-mediated dissociation of water on TiO₂(110),” Nat. Mater. 5:189-192). It has been observed that oxygen adsorption on photocatalyst powders such as TiO₂ and ZnO quenches the photoluminescence (PL) intensity, while adsorption of water produces an enhancement of the PL. Electron-trapping adsorbates, such as oxygen, increase the band-bending of TiO₂, which facilitates the separation of photogenerated electron hole pairs in the oxide. Subsequently, the PL intensity is decreased as the photogenerated charge carries cannot recombine efficiently. Conversely, in the case of water, the band bending is reduced, resulting in an increase in the PL intensity. In explaining the observed behavior of the hybrid sensors, the depletion effect induced by the TiO₂ clusters on GaN NW is considered. Considering an inverse relationship, i.e., increase in depletion of the TiO₂ cluster leads to a decrease in the depletion width in the GaN NW and vice versa, some of the observed sensing behavior is explained.

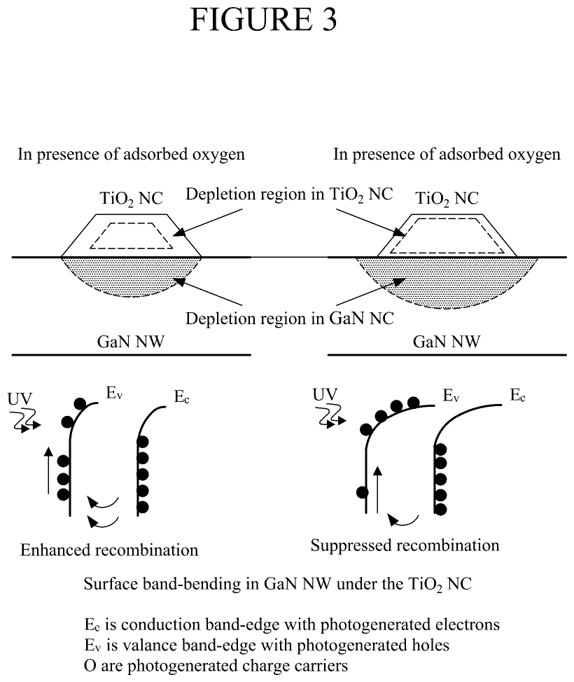

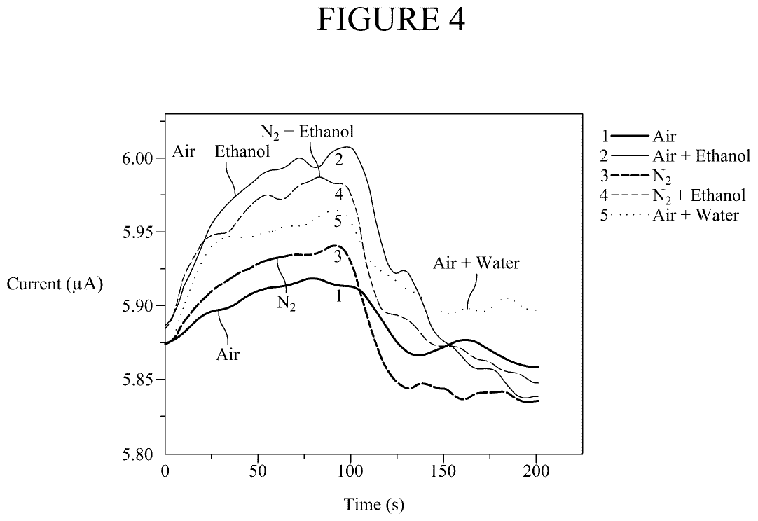

As shown in FIG. 3, when oxygen is adsorbed on the TiO₂ NC surface, the depletion width in the NC increases, leading to a decrease in the depletion width in the NW. Adsorption of water, nitrogen, and alcohol produce the reverse effect: they decrease the depletion width of the TiO₂ NC, leading to an increase in the band-bending on the GaN NW. Increased band-bending in the GaN NW results in an effective separation of charge carriers, leading to an increase in photocurrent through the NW. This qualitatively explains the increase in the photocurrent when the hybrid sensor is exposed to water mixed with air or with pure nitrogen (see FIG. 4). However, the increase in the photocurrent when exposed to 20 sccm of air flow is not fully explained. Under air flow, more oxygen should adsorb on the NCs, causing an increase in the depletion width of the cluster. This should have resulted in a decrease in the photocurrent based on our assumption; however, an increase in the photocurrent is exhibited (FIG. 4) when 20 sccm of air is passed through the chamber.

In the absence of UV light, the absorption or desorption of chemicals from the cluster surfaces cannot modulate the dark current through the nanowire. In the dark, the surface depletion layer of the GaN NW is thicker compared to under UV excitation (see Mansfield L M et al. (2009) “GaN nanowire carrier concentration calculated from light and dark resistance measurements,” Journal of Electronic Materials 38:495-504). The minority carrier (hole) concentration is also significantly lower. Thus the NCs are ineffective in modulating the dark current through the NW.

### Mechanism of Sensing of Alcohols and Hydrogen by GaN/(TiO₂—Pt) NWNC Sensors

Adsorption of alcohols (RCH₂—OH) on the TiO₂ surface leads to their oxidation (Kim K S and Barteau M A (1989) “Reaction of Methanol on TiO₂,” Surface Science 223:13-32). Although there are various mechanisms of oxidation of adsorbed alcohols on TiO₂ surface, focus is on the oxidation of alcohols by photogenerated holes. The process is described by the following reactions:

RCH₂—OH (g) [symbol] RCH₂—OH (ads) (Equation 1)

RCH₂—OH (ads)+h⁺/(photogenerated hole) [symbol] RCH₂—OH⁺(ads) (Equation 2)

RCH₂—OH⁺(ads) [symbol] RCH—OH·(ads)+H⁺(ads) (Equation 3)

RCH—OH·(ads) [symbol] RCHO(ads)+H⁺(ads)+e⁻ (Equation 4)

where (ads) and (g) represent adsorbed and gas phase species, respectively. For Equation 4 to proceed in the forward directions, the H⁺ species should be removed effectively. It is possible that from TiO₂ NCs the H⁺ species can spill-over on to Pt clusters nearby, where they can be reduced to form H₂:

2 H⁺(ads)+2 e⁻ [symbol] H₂(g) (Equation 5)

As H⁺ reduction and hydrogen-hydrogen recombination is weak on the bare TiO₂ surface (Fujishima A et al. (2008) “TiO₂ photocatalysis and related surface phenomena,” Surf. Sci. Rep. 63:515-582), the rate of alcohol oxidation to aldehyde might be affected by the H⁺ reduction and hydrogen-hydrogen recombination on the Pt NCs. Adsorption of alcohols and their subsequent oxidation due to trapping of photogenerated holes leads to a decrease in the band bending of TiO₂ NCs. As shown in FIG. 3, this leads to an increase in the NW photocurrent, which is observed for the GaN/(TiO₂—Pt) sensors when exposed to methanol and ethanol (FIG. 4). It is likely that the production of H.sub.2 on Pt is the key for sensing alcohols by GaN/(TiO₂—Pt) sensors. Additionally, H₂ on Pt surface can dissociate and diffuse to the Pt/TiO₂ interface. Atomic hydrogen is shown to produce an interface dipole layer, which reduces the effective work-function of Pt (Du X et al. (2002) “A New Highly Selective H2 Sensor Based on TiO2/PtO-Pt Dual-Layer Films,” Chem. Mater. 14:3953-3957). Effective reduction of Pt workfunction also reduces the depletion width in TiO₂, which according to the model in FIG. 4, also leads to an increase in the photocurrent when these sensors are exposed to alcohols. In the presence of hydrogen in nitrogen, the workfunction change of Pt NCs due to hydrogen adsorption is the likely cause for the sensing behavior of these sensor hybrids.

Selectivity of GaN/(TiO₂—Pt), GaN/Pt, and GaN/TiO₂NWNC Hybrid Sensors

A significant finding of the present invention is the change in the selectivity of GaN/TiO₂ hybrid sensors due to the addition of Pt NCs. The observed selectivity behavior of the three hybrids can be qualitatively explained if the heat of adsorption of the analytes on TiO₂ and Pt surfaces is considered as shown in Table II and their ionization energies presented in Table III.

TABLE II
Heat of Adsorption for Methanol, Benzene,
and Hydrogen on Pt and TiO₂ (Anatase*)

| | Hydrogen | | Benzene |
|---|---|---|---|
| Surface | (kJ/mol) | Methanol (kJ/mol) | (kJ/mol) |
| TiO₂ | Negligible | 92 | 64 |
| Pt | 100 | 48 | 117 |

*The heat of absorption values for TiO2 rutile surfaces are comparable

TABLE III
Ionization Energy of the Analytes (CRC Handbook of Chemistry
and Physics, 84th ed.; CRC Press: Boca Raton, FL., 2003):

| Organic Compound | Ionization Energy (eV) |
|---|---|
| Methanol | 10.85 |
| Hydrogen | 13.5 |
| Benzene | 9.25 |

Referring to Table II, benzene has a higher heat of adsorption on Pt than on TiO₂. Therefore, benzene will preferentially adsorb on Pt in the TiO₂—Pt cluster. Now, in the absence of Pt, when the benzene is adsorbed on TiO₂ it can interact with the photogenerated charge carriers resulting in the sensing behavior of GaN/TiO₂ devices. However, if benzene is adsorbed on Pt (such as in the case of TiO₂—Pt and Pt nanoclusters on GaN) then benzene molecules cannot interact with photogenerated charge carriers in TiO₂, and therefore are ineffective in producing any current modulation in the nanowire. Thus, benzene is detected by GaN/TiO₂ sensor devices, but not by GaN/(TiO₂—Pt) and GaN/Pt sensor devices.

Further, methanol is detected by GaN/(TiO₂—Pt) sensors only, and not by GaN/TiO₂ and GaN/Pt sensors. From Table III, methanol (unlike benzene) effectively adsorbs on TiO₂, whether Pt is present or absent (as the heat of adsorption of methanol is higher on TiO₂ than Pt). It is believed that methanol on TiO₂ in the absence of Pt does not participate in photogenerated carrier trapping as efficiently as benzene and other aromatic compounds on the TiO₂ nanoclusters. Referring to Table III, the ionization energy of methanol, hydrogen, and benzene is shown. The effectiveness of the process of hole transfer to the adsorbed organic molecules is related to the compound's ability to donate an electron (i.e. the lower the ionization energy of a compound, the easier for it to donate an electron or capture a hole). However, in the presence of Pt nanoclusters nearby, methanol adsorption on TiO₂ ultimately leads to formation of H⁺ through photo-oxidation of methanol, and eventually H₂, which is the key molecule for sensing of methanol by (TiO₂—Pt) NCs on GaN NW. A similar mechanism applies for ethanol sensing by the GaN/(TiO₂—Pt) hybrids.

Hydrogen is detected by GaN/(TiO₂—Pt) and GaN/Pt hybrids, and not by GaN/TiO₂ NWNC sensors, and GaN/(TiO₂—Pt) sensors have a higher response to hydrogen than to alcohols. From Table II, hydrogen has negligible heat of adsorption on TiO₂, thus GaN/TiO₂ devices are not sensitive to hydrogen. However, in the presence of Pt NCs on TiO₂, hydrogen can adsorb on the Pt NCs. Once adsorbed, hydrogen can modify the workfunction of Pt, resulting in a change in the photocurrent through the nanowire. However, this is not the only mechanism, as that would imply that GaN/Pt hybrids should be equally sensitive to H₂. It is believed that when hydrogen is adsorbed on the TiO₂—Pt NC, it also reduces the TiO₂ surface. Thus, in the presence of only Pt on GaN, workfunction modification of Pt solely produces change in the photocurrent in the NW. However, in the presence of Pt and TiO₂ NCs, hydrogen adsorption leads to the modulation of the photocurrent in GaN NW, through modulation of Pt workfunction together with the change in the depletion layer of the TiO₂ NCs, resulting in a larger change of the photocurrent, thus higher sensitivity.

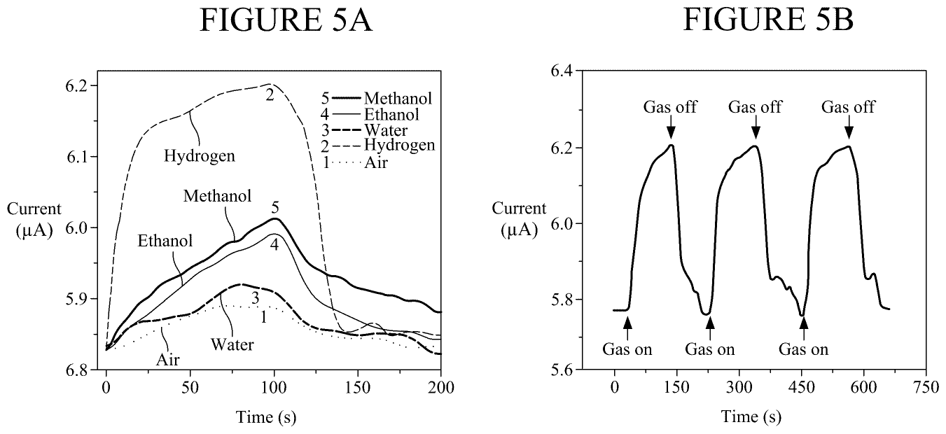

The faster and larger response of GaN/(TiO₂—Pt) towards H₂ compared to the alcohols (as shown in FIG. 5) is due to the fact that in the case of alcohols, hydrogen is produced after photo-oxidation of the adsorbed alcohols, which is a two-step process with lower yield. In the case of H₂ in nitrogen, there is a direct availability of H₂ molecules.

GaN/(TiO₂—Pt) sensors are not sensitive to high carbon-containing (C>2) alcohols such as propanol and butanol. In this regard, it has been shown that the hydrogen production from the photocatalytic oxidation of alcohols on TiO₂/Pt surface is related to the polarity of the alcohols (i.e., the higher the polarity of the alcohol the greater the yield of photocatalytic hydrogen production) (see Yang Y Z et al. (2006) “Photo-Catalytic Production of Hydrogen Form Ethanol over M/TiO2 Catalysts (M=Pd, Pt or Rh),” Applied Catalysis B: Environmental 67:217-222). The polarity (Y) is defined as Y=(εₛ−1)/(εₛ+2), where εₛ is the relative permittivity of the solvent. Table IV lists the polarity of various alcohols tested.

TABLE IV
Solvent Polarity of Various Alcohols

| Solvent | Polarity |
|---|---|
| Methanol | 0.91 |
| Ethanol | 0.89 |
| n-Propanol | 0.86 |
| i-Propanol | 0.85 |
| Butanol | 0.84 |

The relative difficulty of producing hydrogen from higher carbon-containing alcohols (C>2) is believed to be the cause of the GaN/(TiO₂—Pt) sensor's inability to detect alcohols with C greater than 2. The sensor's greater response to methanol than ethanol (at least for concentrations above 500 μmol/mol) is also consistent with the polarities of the alcohols.

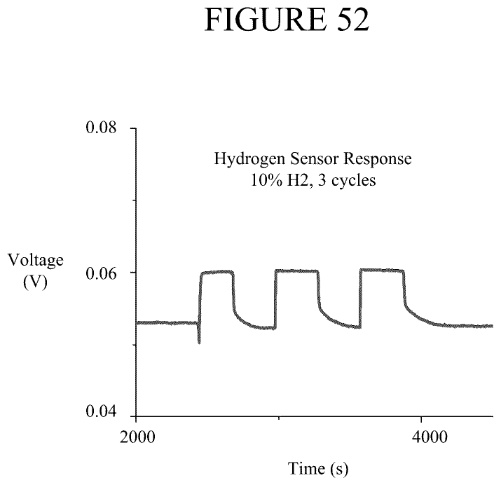

The GaN/(TiO₂—Pt) hybrid sensors are operable at room-temperature sensing of hydrogen, and thus are suitable for various applications (e.g., industrial production facilities, oil refineries, hydrogen monitoring in hydrogen-powered vehicles, alcohol monitoring systems for industrial and law-enforcement purposes, etc.). The disclosed mechanisms and methods may be implemented for achieving other multicomponent NWNC based sensors. For example, the dynamic response of a ZnO—Pd—Ag based sensor exposed to H₂ at room temperature is illustrated in FIG. 52. Through combinations of metal and metal-oxides available, a library of sensors may be produced, each with precisely tuned selectivity, on a single chip for detecting a wide variety of analytes in many different environments.

Thus, an inactive semiconductor nanostructure (e.g., NW) surface may be functionalized with selected analyte-specific active metal-oxide nanoparticles. For example, another embodiment of the present invention provides for alcohol sensors using gallium nitride (GaN) nanowires (NWs) functionalized with zinc oxide (ZnO) nanoparticles. The dynamic response of exemplary ZnO based sensors exposed to benzene (concentration 500 ppm) and hexane (concentration 100 ppm) at room temperature is shown in FIG. 51, plates (b) and (c). The disclosed sensors operate at room temperature, are fully recoverable, and demonstrate a response and recovery time on the order of 100 seconds or less. The sensing is assisted by ultraviolet (UV) light within the 215-400 nm band and with the intensity of 375 nW/cm² measured at 365 nm.

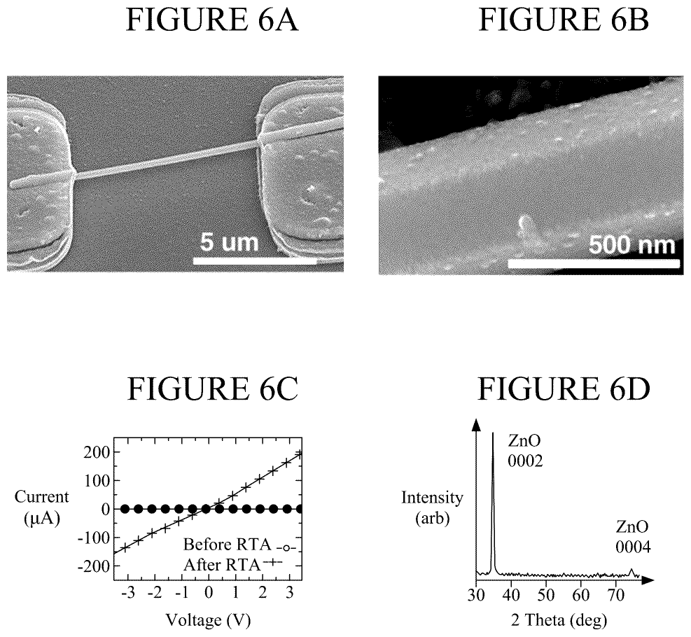

As discussed above, the conductivity model of GaN nanostructure is comprised of a conducting channel surrounded by a surface depletion region, where modulation in the width of the depletion region induces a change in the conductivity of the NW. Similarly, ZnO nanoparticles have a surface depletion layer, which enhances upon exposure to air due to electron capture by surface-adsorbed oxygen. When UV light is turned on, the photogenerated holes in ZnO assist in removing the adsorbed oxygen, thus releasing the electrons captured by surface oxygen back into ZnO. The photoinduced excess of electrons in the ZnO nanoparticles promotes photogenerated charge separation in the GaN nanostructure, thereby resulting in increased conductivity. Conversely, there is a reduction in the number of free electrons in the ZnO nanoparticles when exposed to air, leading to a reduced conductivity. As seen in FIG. 6, this effect increases with increasing flow rate of air due to enhanced coverage of the device surface with adsorbed oxygen.

The device response to alcohols may be explained by the following generic reaction occurring on the surface of ZnO:

2CH₃OH+O⁻₂(adsorbed)→2HCHO+2H₂O+e⁻ (Equation 6)

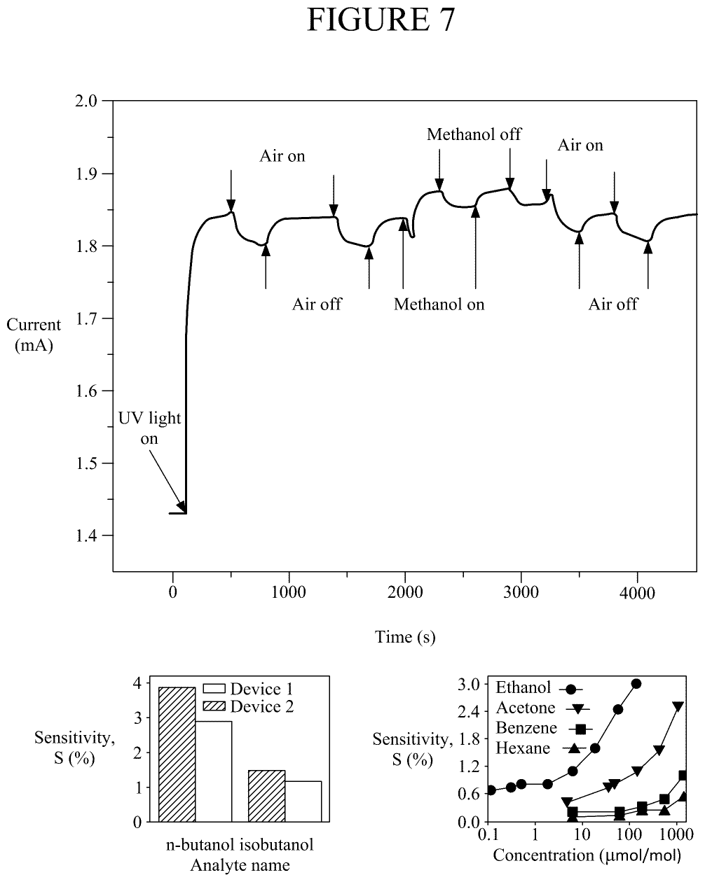

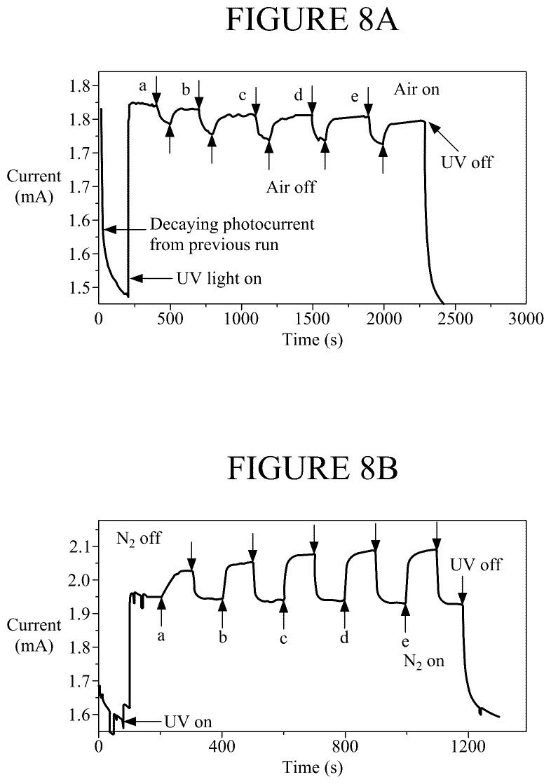

As shown in FIG. 7, the exposure to alcohol vapors leads to increased device conductivity due to the removal of adsorbed oxygen. In the case of exposure to N₂, although there is no surface reaction, N₂ assists in desorption of the oxygen, thus restoring the conductivity, as shown in FIG. 8.

The disclosed hybrid GaN nanostructure/ZnO nanoparticle devices are suitable for UV-assisted alcohol sensing at room temperature. These devices are a suitable candidate for making nanosensor arrays because of their tunable selectivity, ability to detect the pbb level of analytes, and fast response and recovery time.

The disclosed hybrid chemiresistive architectures utilizing nanoengineered wide-bandgap semiconductor backbone functionalized with multicomponent photocatalytic nanoclusters of metal-oxides and/or metals are particularly suitable for larger scale manufacturing techniques, such as for commercial applications. The sensors operate at room-temperature via photoenabled sensing. A substantial benefit of the disclosed sensors is the utilization of all standard microfabrication techniques, thus resulting in economical, multianalyte single-chip sensor solution. By combining the “designer” adsorption properties of multicomponent nanoclusters together with sensitive transduction capability of nanostructured semiconductor backbones, photoenabled, room temperature, ultra-sensitive, and highly selective chemical sensors are achieved.

The sub-micron structures may be formed on an epitaxial thin-film grown on non-conductive/semi-insulating substrate using deep UV lithography and a combination of plasma etching and wet-etching. Such structures are functionalized with multicomponent nanoclusters of metal-oxides and metals using reactive-sputter deposition, as noted above.

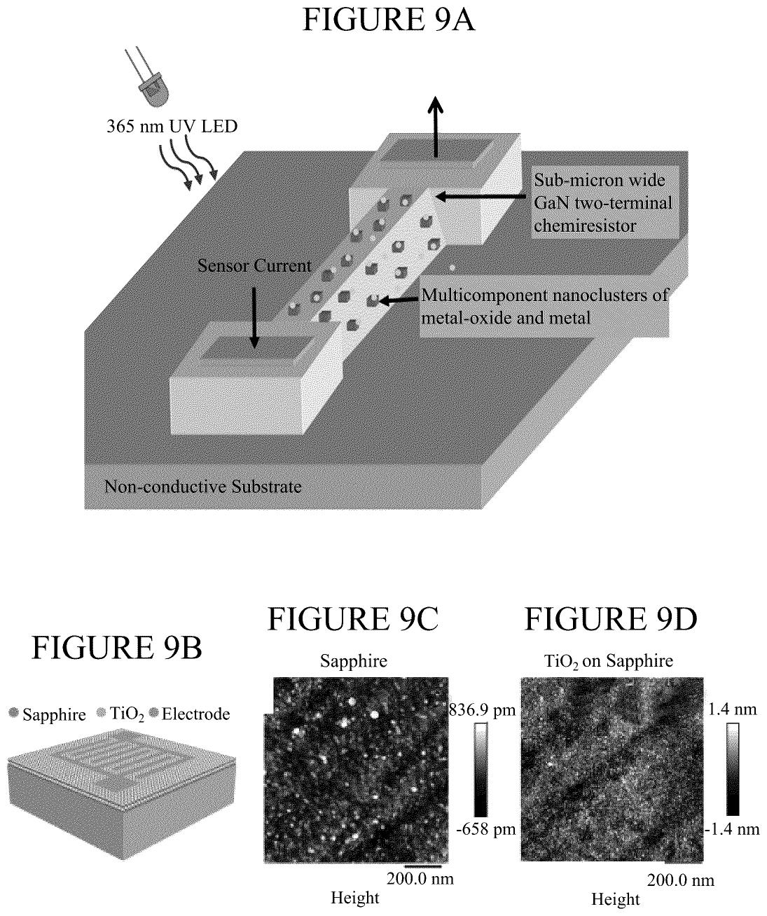

Referring to FIG. 9, an exemplary structure of a semiconductor-nanocluster hybrid sensor is illustrated. Referring to FIG. 9, plate (a), the sensor may comprise a two-terminal sub-micron wide semiconductor backbone, functionalized with nanoclusters of metal-oxides and/or metals. For example, the sensor may include a lightly-doped 0.8-0.25 μm wide semiconductor two-terminal structure on a non-conductive substrate (e.g. sapphire) formed using traditional deep UV photolithography and plasma etching. Functionalization is a discontinuous layer of multicomponent nanoclusters (e.g., each nanocluster comprising one or more photocatalytic metal-oxide nanoclusters (diameter 20 nm and smaller) and smaller metal nanoparticles (5 nm and smaller) deposited on top of it). The multicomponent design may include more than one oxide and metal types in the nanoclusters, and exhibits tailored adsorption properties by virtue of the multicomponent design. The functionalization layer is deposited using reactive sputtering technique followed by thermal treatment—all standard semiconductor microfabrication processes. The sensors work with low-intensity light, such as from an LED. The emission wavelength is determined by the semiconductor and metal-oxide bandgaps. FIG. 9, plate (b) illustrates schematically an exemplary thin-film device including a semiconductor backbone functionalized with TiO₂ on a sapphire substrate. The smoothness of the substrate and film after thermal processing is shown in FIG. 9, plates (c) and (d).

Surface defects of metal-oxides are the active sites for adsorption of various chemicals. However, at room-temperature the adsorbed oxygen and water are very stable. This necessitates heating in traditional metal-oxides sensors. Most metal-oxides are well-know photocatalysts, with photoexcitation wavelengths in the range of ultraviolet to visible, corresponding to the material bandgap. A disclosed approach uses dynamic surface-defects generation in the metal-oxide cluster through illumination, which allows for efficient photodesorption of adsorbed water and oxygen. This has at least two benefits: 1) low-power, room-temperature operation, which also increases the lifetime of the sensors, and 2) real-time dynamic range modulation by changing the intensity of light (for ppt level detection the intensity of the LED can be increased as compared to ppm level detection).

The sensor architecture provides for the combination of a crystalline top-down fabricated semiconductor backbone with a discontinuous nanocluster surface layer. In metal-oxide gas sensors, the resistance changes due to diffusion and adsorption of gases along the grain boundaries. As the present architecture uses a discontinuous, nano-island like metal-oxide layer, the bottleneck of gas diffusion through grain boundaries, as in traditional metal-oxide sensors, is not present. This makes the disclosed sensors respond relatively fast as compared to conventional sensors, and operable at room-temperature. Unlike traditional metal-oxide sensors, the disclosed design provides that the current is carried by the high-quality, high mobility semiconductor backbone, which makes the sensor fast. Also, the absence of conduction in the nanocluster layer makes the active layer inherently stable as compared to traditional metal-oxide thin film sensors (e.g., grain boundary motion, defect generation and propagation, and reduction of the metal-oxide layer is not possible due to the absence of a “closed-circuit”).

Due to the nanocluster layer of the disclosed sensors, designed with a specific adsorption profile, they are extremely efficient in adsorbing target analytes. This enables the design of highly-selective sensors. Two component, three component, four component, or five or more component cluster designs are possible for unprecedented selectivity tailoring.

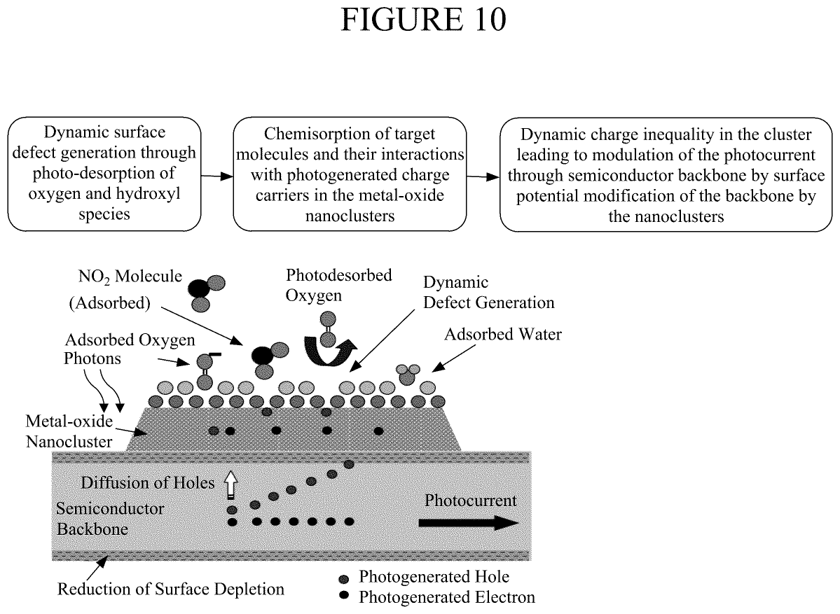

Most semiconductors have depletion regions associated with them. The surface band bending, which is a consequence of the surface depletion, facilitates the diffusion of the photogenerated holes to the surface. This separation of carriers effectively suppresses their recombination. The degree of separation is determined by the surface potential modification by the clusters. Such separation of photocarriers increases their lifetimes, leading to higher photocurrent and thus sensitivity towards such surface potential modifications. The processes that enable sensing of different adsorbed molecules with the disclosed multicomponent nanocluster functionalization is shown schematically in FIG. 10.

Assuming typical values of the response/recovery times for 500 ppt of NO₂, from the kinetic theory of gases the flux F of NO₂ arriving on a surface is given by the formula:

$$F = \frac{{N}_{A} {P}_{partial}}{\sqrt{2 π MRT}} \qquad ( Equation 7 )$$

where N_{A} is the Avagadros' number, M is the average molar weight of the molecule, P is the pressure, T is the temperature, and R is the gas constant.

For 500 ppt concentration of NO₂ in air, three molecules of NO₂ are impinging on a 20 nm diameter metal-oxide cluster per second. Now, the residence time t of an adsorbate at temperature T on a surface is given by the relation τ=τ0 exp (ΔH_{ads}/RT), where ΔH_{ads} is the heat of adsorption, and TO is correlated with surface atom vibration (roughly 10⁻¹² s). Thus, at 298 K the residence time for NO₂ molecule on WO₃ nanocluster is approximately 15 seconds (considering ΔH_{ads} for NO₂ on WOs to be 18 kcal/mol). Considering roughly 10²¹ cm⁻³ of defect density for typical metal oxides, results in roughly 300 adsorption sites on a 20 nm diameter nanocluster. Assuming sticking coefficient of 1, by 110 seconds the surface defects are saturated. Thus, response time may be estimated to be in the order to 100 seconds, and recovery time in the order to 15-30 seconds. Although the design of the nanocluster is described from pure thermodynamic standpoint, other surface kinetics (such as diffusion, desorption) may also be considered.

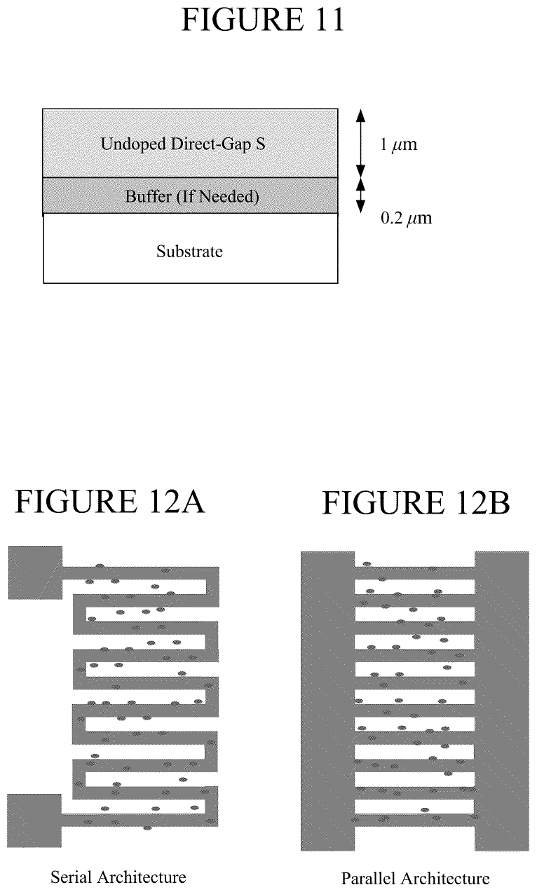

For fabricating the sensor backbone, un-doped (1×10¹⁶ cm⁻³) to lightly doped (1×10¹⁷ cm⁻³) semiconductor epitaxial layer (1 μm thick) on sapphire/insulating/semi-insulating substrates may be utilized, as shown in FIG. 11. Lower doping is needed for the sensors to be photo enabled. The thickness of buffer layer controls the defects arising from lattice and thermal mismatch. Ideally suited layer structures require a relatively thin buffer layer (e.g., about 250 nm) to suppress the parasitic conduction in the buffer layer. Similar designs may also be provided with other direct gap semiconductors, such as ZnO, InN, AlGaN and virtually any other direct gap semiconductor material.

The design of submicron semiconductor backbone including physical layout and geometry is described with reference to FIG. 12. Both serial and parallel architectures for the semiconducting resistive backbone have unique advantages and disadvantages as the chemiresistor backbone. Serial architecture has higher resistance which results in lower-power operation, whereas parallel architecture produces more robust devices insensitive to material quality variation in the individual sections. However, the calculation will show that the response R is the same for both serial and parallel architecture:

$$R = \frac{{R}_{analyte} - {R}_{air}}{{R}_{air}} \qquad ( Equation 8 )$$

wherein R_{analyte} and Rₐᵢᵣ are the resistances in presence of analyte and in air, respectively. However, the resolution of the sensor (i.e., smallest change in concentration it can measure as required for proposed large dynamic range sensors) is greater in a serial architecture.

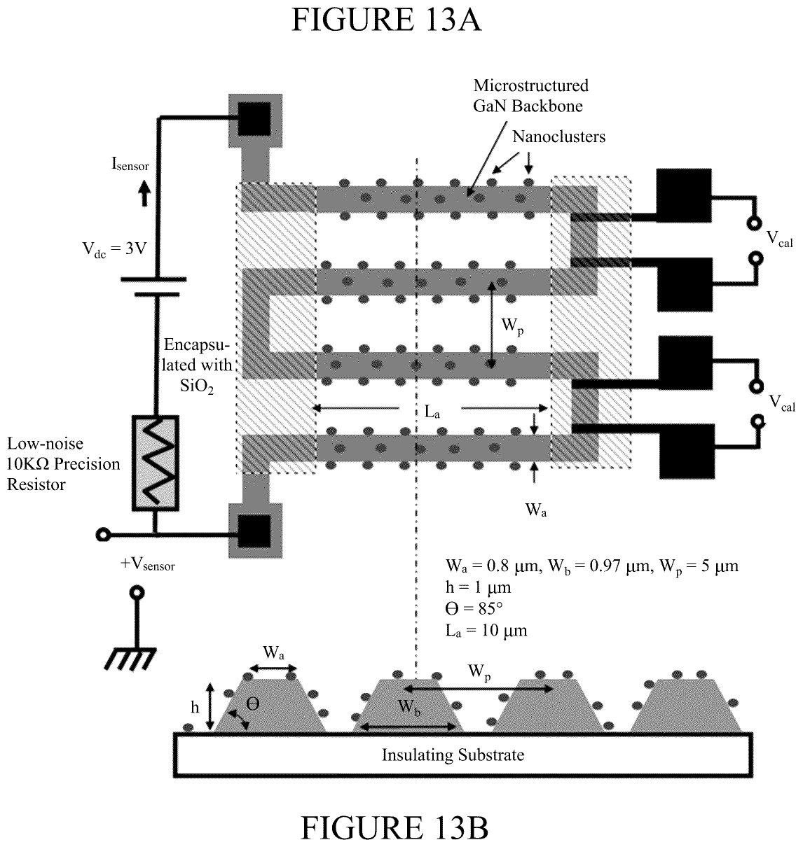

The series sensor element provides for a meander shape, with integrated passive sections as real-time calibration elements. An exemplary design is shown in FIG. 13, plate (a). The surface area-volume ratio for this structure is roughly 3.1. The sidewalls of the backbone may be intentionally angled, such as at 85° as shown in FIG. 13, plate (b). This ensures uniform coverage of the nanoclusters on the sidewalls of the structure, and also ensures uniform photoexcitation of the semiconductor backbone. The device is biased by a standard three dc voltage source (two AA batteries in series) and the sensor output is the voltage measured between the pads+Vₛₑₙₛₒᵣ and ground. The design provides various benefits including: 1) high sensitivity and resolution; 2) low-power consumption; 3) simplified interface circuit; and 4) ability for real-time base-line drift calibration and temperature compensation even in presence of analytes.

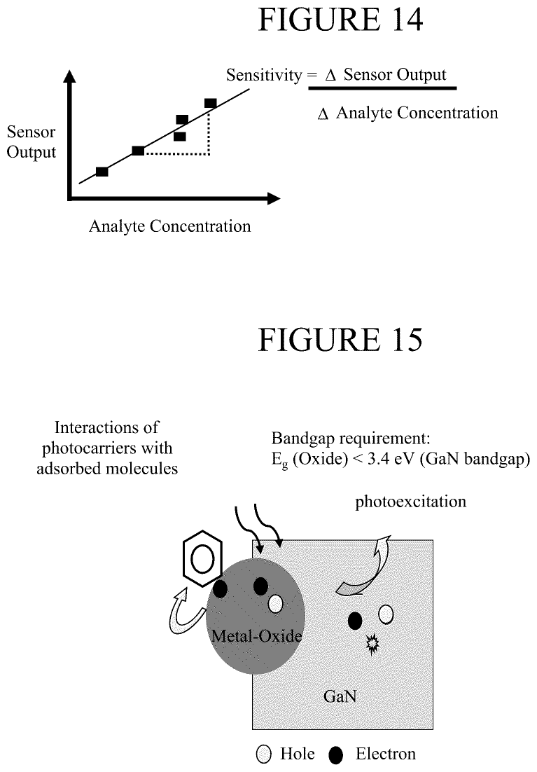

Using circuit analysis, it can be shown that Sensitivity S (as defined in FIG. 14) may be simplified considering R_{L}<<R as:

$$S = \frac{{R}_{L} \times {V}_{dc}}{NR} [ \frac{1}{\frac{R}{Δ R} + 1} ] \qquad ( Equation 9 )$$

wherein R_{L} is the external low-noise precision load resistance (e.g., see FIG. 13, plate (a)), Nis the number of segments, R is the resistance without analyte of single segment, and ΔR is the resistance change of the single segment in presence of the analyte, and V_{dc} is the dc source voltage.

Thus for higher sensitivity, N should be small, and R_{L} and V_{dc} should be large. However, resolution of a sensor is the smallest change in concentration of the analyte it can measure (it is different from lowest detection limit), and is often limited by the noise. Considering only thermal noise current in the total sensor, the output sensor voltage noise can be expressed as:

$${V}_{sensor} ( noise ) = {R}_{L} \sqrt{\frac{4 {k}_{B} T Δ f}{NR}} \qquad ( Equation 10 )$$

wherein k_{B} is the Boltzmann Constant, Tis the temperature, and Δf is the bandwidth. Considering both Equations 9 and 10, the tradeoff between high sensitivity and resolution is clear. The effect of N (i.e., number of segments) on the sensor performances such as sensitivity, detection limits, and resolution, may be investigated.

Referring again to FIG. 13, plate (a), the resistance of the active sensor area may be computed using the formula, neglecting the bends:

$$R \approx \frac{ρ \times ( 4 \times {L}_{a} )}{h \times ( {W}_{a} + {W}_{b} ) / 2} \qquad ( Equation 11 )$$

wherein ρ=1/(nqu), ρ is the resistivity, n is the carrier concentration, and μ is the mobility (see also dimensions shown in FIG. 13, plate (b)).

For example, for the GaN backbone with dimensions shown in FIG. 13, the active-area photoresistance under 365 nm excitation from LED is =60 kΩ, assuming a mobility of 300 cm²V⁻¹ s⁻¹ and electron concentration of 1×10¹⁷ cm⁻³. The device is considered to be excited by low-intensity (10 W/cm²) 365 nm LED. The GaN absorption coefficient α=105 cm⁻¹ for the 365 nm photon is assumed. If the sensor is biased with 3 V dc and with an external 10 KΩ resistor, the power dissipation is approximately only 40 μW. The sensor power dissipation when in offstate (LED turned off and the sensor has only dark current) is even lower. The total power requirement for the sensor must also include the power required for LED operation. There are several low-power UV (365 nm) LEDs (FOX GROUP) that could be run by LED drivers. Power dissipation for the LED could be low as 0.5 mW, if we drive the LED for very low intensity. Using a LED driver to control the intensity has an added benefit of the real-time dynamic range configuration.

The simplified chemiresistive architecture lends itself easily to integration with interface devices as compared to more complex devices such as metal-oxide-semiconductor field-effect transistors (MOSFETs). The nano-watt operation amplifier (OP-Amp) TS1001 from Touchstone Semiconductor is identified, which can provide a gain of 100 when operated in single-input voltage amplifier configuration. The Op-Amp operated from a single AA battery dissipated about 1 μW.

In one implementation, a feature of the present design is the inclusion of the voltage probes (V_{cal}) for calibration of base line drift of the photoresistance of the total structure. As the area under the calibration probes is encapsulated with thick SiO₂, the voltage drop (V_{cal}) for a fixed intensity of illumination through the entire structure will enable compensation for drift in the baseline photoresistance arising from persistence photoconductivity or temperature-induced drift.

Another feature of the present design is the “tailored” adsorption profile through the multicomponent nanocluster design, as described above. The design provides for suppressing the competitive adsorption of an interfering chemical on a surface with two different adsorption profiles, which is achieved using a primary and a secondary component.

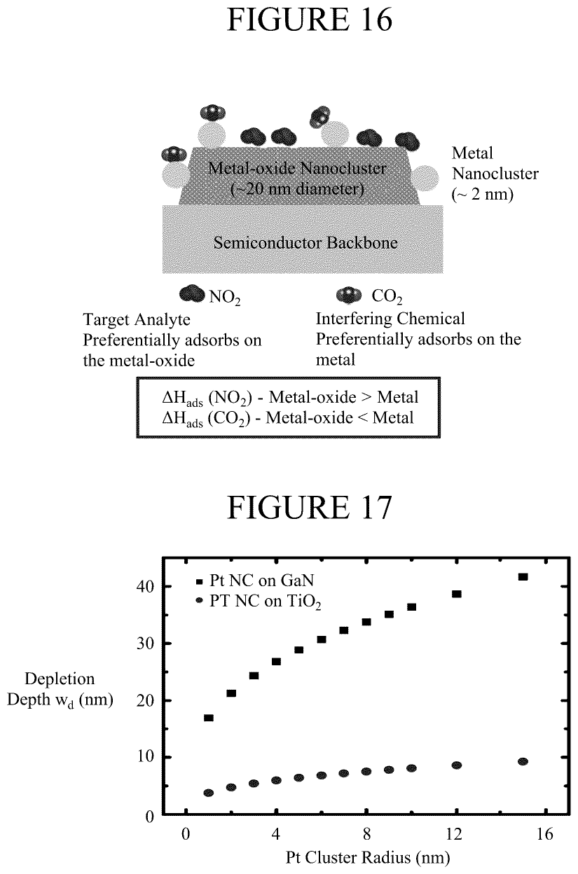

In this regard, FIG. 16 illustrates an exemplary multicomponent design for the target analyte of NO₂ and for the interfering chemical of CO₂. Adsorption profile for another target analyte or set of analytes along with a set of interfering chemicals may alternatively be provided utilizing a similar configuration. The primary metal-oxide component is chosen so that the heat of adsorption of NO₂ on its surface is large compared to CO₂. The secondary component (e.g., the metal) is chosen with the heat of adsorption for CO₂ larger than the metal-oxide. Thus NO₂ and CO₂ preferentially adsorb on the metal-oxide and the metal, respectively. When NO₂ is adsorbed on the metal-oxide, it interacts with the photogenerated charge carriers, producing modulation of the semiconductor backbone photocurrent, as explained above. However, when CO₂ is adsorbed on the metal, due to the large concentration of electrons, there is minor change in the cluster potential. Consideration of other effects, such as catalytic decomposition on the metal, spill-over from the metal to metal-oxide, and change of metal-work function due to adsorption of gases, may also be appropriate.

Due to the highly dispersed nature of the metal phase, even if there is a change in the physical properties of the metals, it has only marginal impact on the cluster properties. Although the general design principles are described, the specific designs of the appropriate clusters may be fine-tuned for optimal performance and selectivity. For example, Table V below demonstrates possible cluster designs for NO₂ and benzene sensing. Considering the heat of adsorption of NO₂ on WO₃ and Pt, bigger WO₃ clusters with much smaller and dispersed phase of Pt may be favorable. Although, adsorption energy for NO₂ is comparable on both WO₃ and Pt, due the higher surface area of metal-oxide clusters, most of NO₂ will adsorb on the WO₃, whereas CO₂ will mostly adsorb on the Pt. For BTEX sensing, the TiO₂/Fe is favorable.

TABLE V
Heat of adsorption on different candidates
for the multicomponent cluster design.
Possible Cluster Designs for NO₂ sensing:

| | NO₂ | CO₂ |
|---|---|---|
| Metal-Oxide/Metals | (kcal/mol) | (kcal/mol) |
| MgO | 9.0 | 3.5 |
| TiO₂ | 21.0 | 29 |
| WO₃ | 18.4 | negligible |
| Fe(III) | 64.5 | 69 |
| Pt(III) | 19 | 40.5 |

Possible Cluster Designs for Benzene sensing:

| | Benzene | CO₂ |
|---|---|---|
| Metal-Oxide/Metals | (kcal/mol) | (kcal/mol) |
| TiO₂ | 15.2 | 29 |
| Fe (III) | 22 | 69 |

Note that the values in Table V are average adsorption energies at room temperature for low adsorbate coverage. The values are collected from experimental results (temperature programmed desorption and calorimetric studies) and theoretical calculations (such as density function theory). The values shown are for common and stable oxide surfaces. Experimental heat of adsorption values are dependent on various factors, including the morphology and crystal orientation of the surface.

Other design considerations for the nanoclusters include:

1) Bandgap of the oxide: as single wavelength excitation is used for both photodesorption of surface oxygen and hydroxyl species, and for creating photocarriers in the semiconductor (e.g. GaN), the bandgap of the oxide should be lower or equal to GaN bandgap (as shown in FIG. 15). Candidates are shown in Table VI below.

TABLE VI
Bandgaps of Common Metal Oxides

| Metal-Oxides | Bandgap (eV) |
|---|---|
| MgO | 7.1 |
| TiO₂ | 3.2 |
| WO₃ | 2.8 |
| Fe2 O3 | 2.1 |
| V2 O5 | 2.3 |
| NiO | 3.6 |
| Al₂O₃ | 7.0 |

Candidates are in bold and underlined, E_{g} < 3.4 eV.

2) Nature of surface defect types: surface defects (i.e. the active adsorption sites) of metal-oxides are of three types: bronstead, lewis-acid/base sites, and redox sties. Organic compounds such as benzene predominantly adsorb by dehydgrogenetion (i.e., removal of H+) requiring surface lewis sites. On the other hand, NO₂ predominantly adsorbs as surface nitrate (NO₃⁻), requiring base sites. Most metal-oxide surfaces at room-temperature are hydroxylated, and thus photoexcitation will increase the concentration of one type of predominant defects.

3) Redox potentials of the oxide: redox potentials of oxides indicate the ability of photogenerated carriers to oxidize or reduce any adsorbed molecule. Depending on whether molecules will be oxidized or reduced on the surface, they interact with charge carriers differently in the clusters.

4) Stability of the adsorbates: Stability of the adsorbed species is an important consideration, as it determines the recovery time, and ultimately usability of the sensors. As can be seen for Fe, where the very high adsorption energy might produce very stable NO adsorbed species on the surface, rendering the nanoclusters inactive after exposure to high concentrations of NO₂.

5) Nature of the adsorbed species (molecular or dissociative): nature of the adsorbed species determines the photochemical reaction pathways and ultimately the sensitivity. Additional multicomponent nanocluster designs for NO₂ and BTEX sensing are shown in Table VII.

TABLE VII
Possible designs of nanoclusters

| Metal-Oxides/Metal | Target Analyte |
|---|---|
| WO₃/Pt | NO₂ |
| TiO₂/Fe | BTEX |

The use of heterogeneous metal-oxide supported metal catalysts in industrial production, abatement, and remediation for the past few decades has been extensive, and generated an exhaustive body of literature that may be readily utilized for nanocluster designs according to the present invention. Indeed, some of the systems are well-understood, so that a desired selectivity outcome may be readily predicted. The well-known strong metal/support interactions (SMSI) effects in heterocatalysts are different, as the metals are not reduced on the oxides in the disclosed devices.

Computing the size and coverage of the clusters is an important consideration, given the size and coverage of the NCs ultimately determines the overall sensitivity of the device. Thus, determination of the most effective size and coverage of the clusters is desirable. It is known that the surface area and relative particle size has a significant effect on the catalytic properties of metals and metal oxides. However, due to the presence of metals on the metal-oxide clusters, there will be significant depletion of the metal-oxide clusters. Thus, overly small metal-oxide clusters would be substantially depleted and hamper effectiveness, whereas overly large clusters would also result in lower sensitivity. Consideration of the nature of the depletion regions formed by such nano-sized metal clusters on a semiconductor is therefore prudent.

The classical Schottky model depletion theory cannot predict accurately the zero-bias depletion width produced by metallic nanoclusters on a semiconductor. According to Zhdanov's model, the depletion depth associated with such metal nanoclusters on a semiconductor can be estimated by the following relationship:

$${w}_{d} = {( \frac{3 {r}_{c} {V}_{bi}}{2 π {q}^{2} {N}_{d}} )}^{1 / 3} \qquad ( Equation 12 )$$

wherein w_{d} is the depletion width, r_{c} is the radius of the nanocluster, V_{bi} is the built-in voltage for the junction, q is the elementary charge, and N_{d} is the dopant concentration in the semiconductor.

The plot in FIG. 17 demonstrates the depletion width of TiO₂ clusters due to Pt particles. It is clear that 4 nm of Pt clusters on 20 nm diameter TiO₂ clusters would produce depletion of about 5 nm in the TiO₂.

Coverage of the metal-oxide nanocluster functionalization is determined by the limit of formation of continues metal-oxide film. The coverage is dependent on various parameters such as metal-oxide wetting of the semiconductor, morphology of phases formed after thermal treatment, etc., and may be verified by SEM imaging. The metal coverage should be sparse to ensure only partial depletion of the clusters.

With regard to fabrication, techniques such as wet chemical etching may not be suitable for etching nanoscale, high aspect-ratio nanostructures due to undercutting of the mask and sloped sidewalls. Hence, the development of a dry etching process with relatively less low damage and precise-depth control capability is preferred for the fabrication of nanostructures. Such etching of semiconductor nanostructures is described in further detail in Example 4 below.

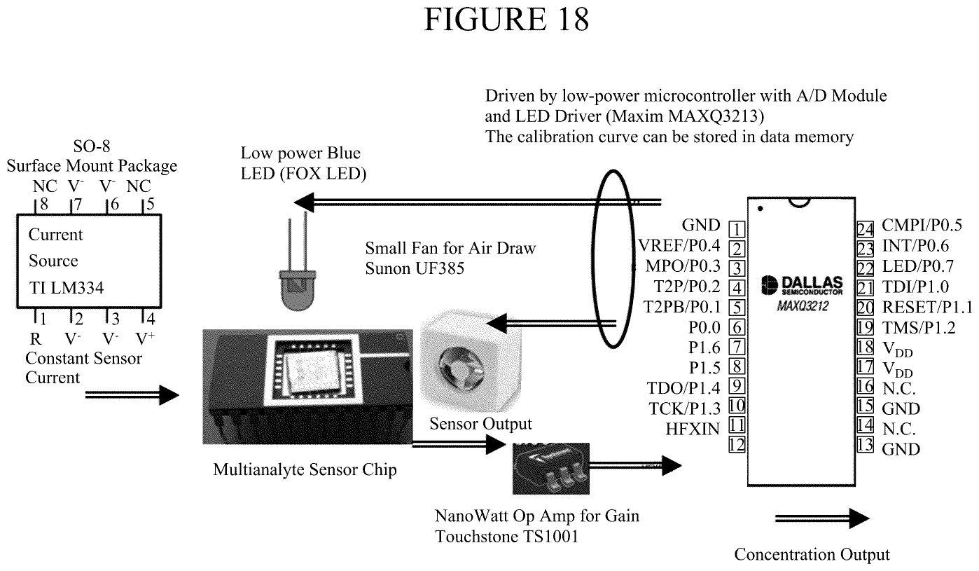

Referring to FIG. 18, the components for an exemplar interface circuit is illustrated. The LED intensity may be controlled by the microcontroller (MAXQ3213, with a LED driver). By relatively simple design change of a selected multicomponent cluster, different applications are readily provided. In addition, using wide bandgap material as a backbone enables the sensor to work at elevated temperatures, and in presence of radiation and other harsh environmental conditions.

As shown in Table VIII below, the possible designs of the multi-component nanoclusters are virtually unlimited, resulting in the ability to provide sensors for numerous applications.

TABLE VIII
Exemplary Designs of Multicomponent Nanoclusters

Nanocluster Components:

| Semiconductor | Metal Oxide: | Metal: |
|---|---|---|
| GaN | Titanium Oxide | Titanium |
| InN | Tin Oxide | Nickel |
| AlGaN | Iron Oxide | Chromium |
| | Magnesium Oxide | Cobalt |
| | Vanadium Oxide | Ruthenium |
| | Nickel Oxide | Rhodium |
| ZnO | Zirconium Oxide | Gold |
| InAs | Aluminum Oxide | Silver |
| | Copper Oxide | Platinum |
| | Zinc Oxide | Palladium |
| | Strontium Oxide | Vandium |

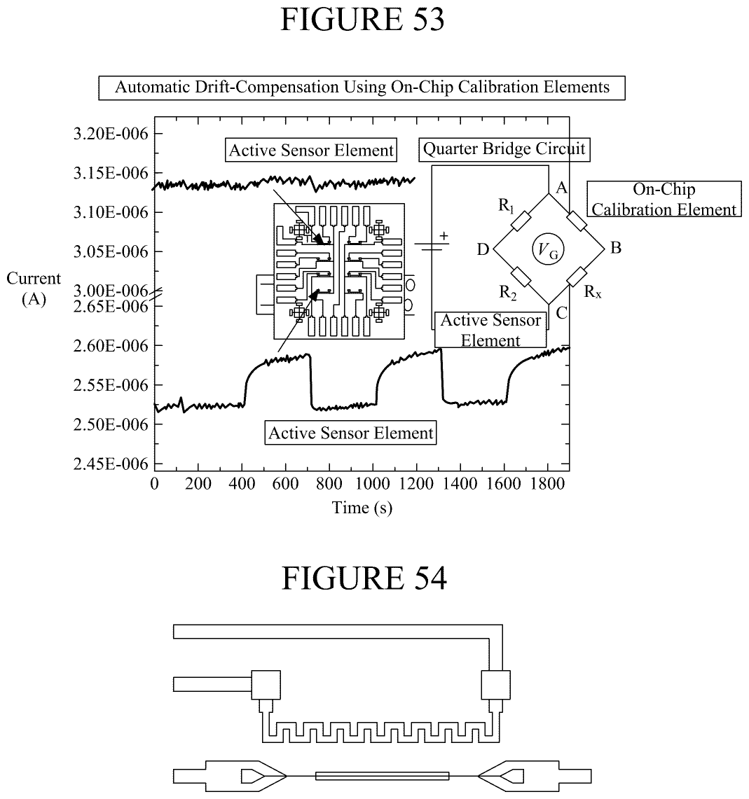

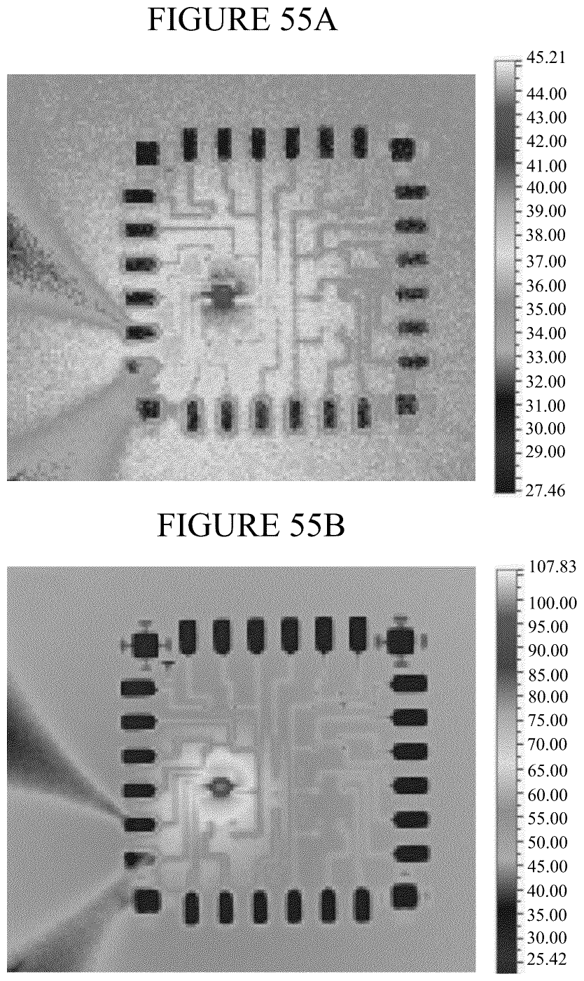

Thus, in accordance with the disclosed methodologies, sensor devices suitable for a wide range of applications are achieved. Further, the particular architecture of the sensor devices may be readily tailored for the desired application and associated conditions, as well as one or multiple active sensor elements configured for sensing particle targets. For example, an exemplary sensor device includes eight individually addressable active sensor elements, as shown in FIG. 53, which can each detect a different target analyte (e.g., various gases). The sensor device may include an on-chip calibration element for automatic drift compensation. The sensor device may also include an on-chip micro-heater, as shown in FIG. 54, for stabilizing temperature and/or humidity. The temperature profiles of a 50 μm microheater made from a Ti/Ni metal stack MH recorded at 5 V bias voltage and 10 V bias voltage are shown in FIG. 55, plates (a) and (b), respectively.

Thus, the disclosed sensor devices may comprise various active sensor elements and passive elements for formation of on-chip circuits. Multiple active elements may be provided with a combination of different functionalization to detect multiple gases in a single chip. The chip may include precise passive elements (elements which have the same semiconductor backbone but passivated from the environment), for calibration on the same chip, which has the same temperature coefficient for current as the active sensor element. Thus, any change due to the temperature or aging can be a calibrated out using the on-chip calibration element(s). Using such on-chip components (e.g., see FIG. 53), bridge circuits may be provided directly on the chip, allowing for sensor devices with high resolution.

Although the sensor devices may comprise a micro-heater element as noted above, such element is not required. The disclosed sensor devices do not need to be heated for sensing, and are capable of sensing a host of gases at room temperature. Total power consumption is extremely low (e.g., an exemplary 8 active sensor element device provided for a total power consumption about 10 microwatts. Further, the disclosed sensor devices are stable and recoverable even in the presence of corrosive gases (e.g, HCN, CL₂, HCl, etc), and capable of withstanding very high gas concentrations. The sensor devices are also capable of operating in oxygen rich or relatively lean conditions.

In accordance with disclosed embodiments, the active sensor(s) elements are designed by first selecting a nanoclusters and/or a layer of a base photocatalytic metal oxide (e.g., TiO₂, V₂O₅, Cr₂O₃, Fe₂O₃, CoO, NiO, CuO, ZnO, ZrO₂, WO₃, MoO₃, SnO₂). Nanoclusters of a catalytic metal (e.g., Ti, V, Cr, Fe, Co, Ni, Cu, Al, Zr, Nb, Mo, Ru, Rh, Pd, Ag, Hf, Ta, W, Re, Ir, Pt, Au) are then applied on top of the base photocatalytic metal oxide nanoclusters. Alternatively in other embodiments, nanoclusters of a second photocatalytic metal oxide different than the base metal oxide are applied on top of the base metal oxide, providing for dual metal oxide functionalizations. Thus, the sensor element comprises a base layer or nanoclusters of a first metal-oxide, and nanoclusters of a second metal oxide or metal. The selection of the particular metal oxide and metal provides for the desired selectively.

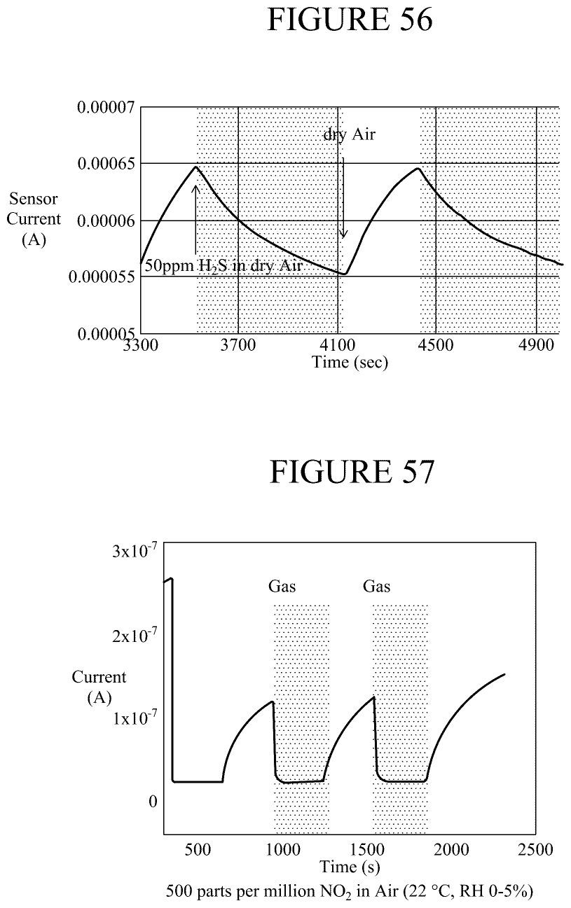

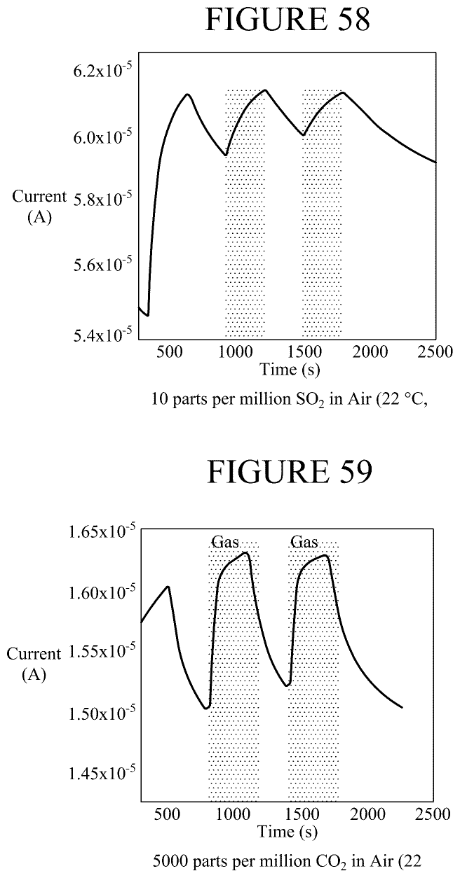

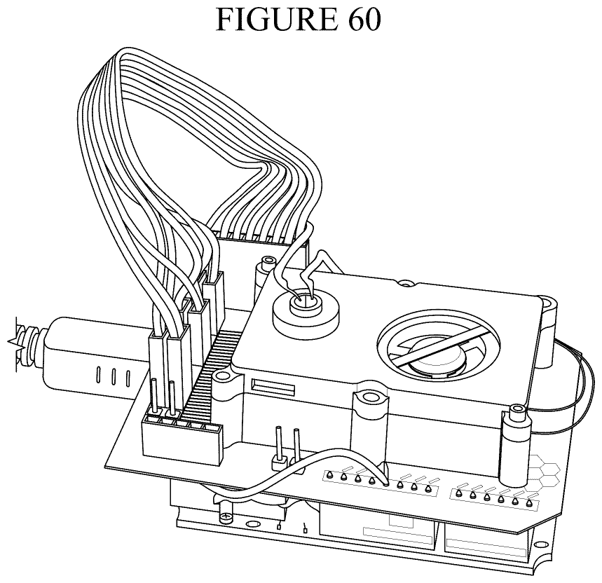

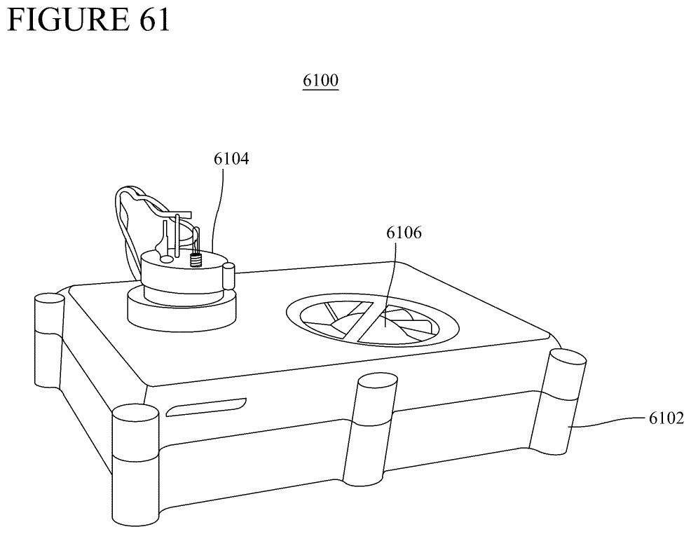

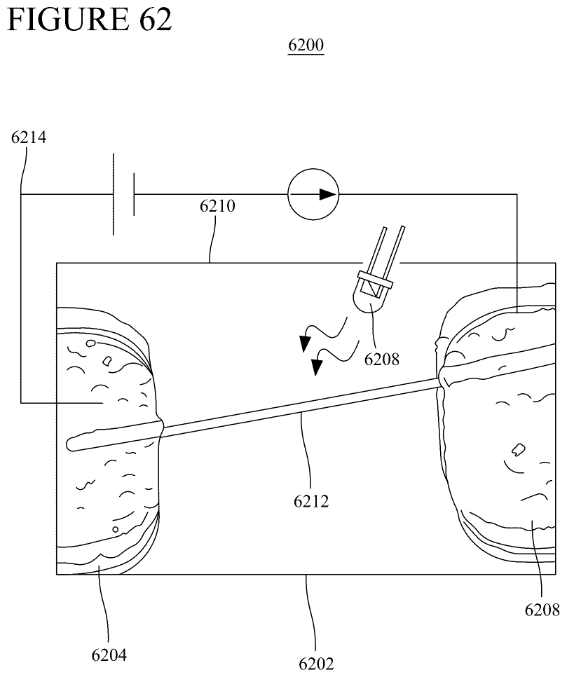

For example, the dynamic response of functionalized GaN NW with selected metal oxide for selectively sensing hydrogen sulfide (H₂S) in dry air is shown in FIG. 56. The response of the functionalized GaN NW for sensing NO₂ in dry air is shown in FIG. 57. The response of the functionalized GaN NW for sensing SO₂ in dry air is shown in FIG. 58. The response of the functionalized GaN NW for sensing CO₂ in dry air using the metal oxide sensor devices of the present invention is shown in FIG. 59. Thus, a wide range of target gases, from reducing to oxidizing to inert gases, is achieved.

A summary of operational and performance specifications of sensing devices in accordance with disclosed embodiments is set forth in Table IX below:

| | | |
|---|---|---|
| | | Response (%) = |
| Analyte | Range of Detection | (R_{gas} − Rₐᵢᵣ /Rₐᵢᵣ) |

| | | | |
|---|---|---|---|
| Ammonia | 1-100 | ppm | 15 |
| Chlorine | 0.5-10 | ppm | 212 |
| Hydrogen chloride | 1-100 | ppm | 74 |
| Hydrogen cyanide | 1-100 | ppm | 10 |
| Hydrogen sulphide | 10-1000 | ppm | 20 |

| | | |
|---|---|---|
| Hydrogen | 0.5-10% | 500 |
| Oxygen | 10-30% | 40 |
| Carbon dioxide | 01.-1% | 2 |

| | | | |
|---|---|---|---|
| Carbon monoxide | 10-300 | ppm | 15 |
| Nitrogen dioxide | 100-500 | ppm | 2 |
| Nitric oxide | 5-1000 | ppm | 2.6 |
| Methane | 50-5000 | ppm | 9 |

The disclosed devices are suitable for environmental monitoring, hazmat, large-scale industrial monitoring and control, explosive threat detection, and other markets where rapid detection of gases and chemicals in air is desired. Compared to conventional sensors, the disclosed sensors of the present invention are extremely small (e.g., 4 mm×4 mm, or 2.5 mm×2.5 mm, or smaller) and inexpensive, exhibit low power consumption (e.g., less than 100 microwatts, and in some embodiments less than about 10 microwatts), but capable of sensing a large dynamic range (e.g., 100 parts per billion to >2%), detect a variety of chemicals under various conditions with no cross-sensitivity (thus minimizing false positives), and exhibit a long operating life. In addition, the disclosed sensors of the present invention may be manufactured using the same manufacturing methodologies utilized for producing conventional integrated circuits. An exemplary sensor module is shown in FIG. 60, which has dimensions of about 8 cm×6 cm×1 cm, a weight of 0.4 pounds, power consumption requirements for continuous operation of about 0.2 watts, eight active sensor elements or channels for simultaneous measurement of eight different target gases, and including a built-in air sampling element with microblower.

The disclosed sensor devices may be installed in residential and commercial buildings for on-demand ventilation control, resulting in a decrease in energy consumption. The sensors can detect the presence of harmful VOCs (Benzene, Xylene, and formaldehyde), which are often emitted by building materials, paints, and furniture, and are also associated with human metabolism. After detecting an increase in the levels of targeted harmful chemicals, the ventilation system may be adjusted for safety, comfort and health of the occupants. Alternatively or in addition, the sensors could monitor CO levels and gas leaks in buildings for safety. Thus, the disclosed sensor technology may be readily implemented in indoor monitoring systems, thereby generating large cost savings in terms of energy efficiency, health of the occupants, and low-maintenance costs.

In case of accidental release of chemicals, the disclosed sensors are suitable for use by first-responders to detect the presence of chemicals and associated hazards. Thus, the challenges of a disaster may be managed more safely and efficiently. The disclosed hybrid sensor technology may be implemented in ultra-small, handheld units, which identify multiple hazardous materials with low power consumption. Such devices would be ideal for first responders.

The disclosed sensors are also suitable for industrial monitoring applications. For example, the sensors may be used for monitoring different gases for process control in industrial facilities such as oil refineries, manufacturing plants, etc. They may be installed at various points throughout an industrial facility for point detection for leaks of toxic chemicals. The may also be implemented in personal monitoring devices for recording personal exposure levels for compliance purposes with state and federal maximum exposure level regulations. The disclosed technology therefore promises unlimited control over the sensor design, thus having the ability to produce sensors for various different industries and processes.

Implementations of the disclosed technology for law enforcement and safety applications are also provided. For example, the disclosed sensors may be utilized in breath analyzers for law-enforcement and individual use. The hybrid sensors may also be integrated into hand-held devices (e.g., cell phones) as plug-in modules to existing devices. For example, the disclosed sensor may be integrated into a hand-held device to enable a user to check his or her blood alcohol level.

Implementations of the disclosed sensor technology are also suitable for defense and security applications. The sensors may be used for safety monitoring in public places such as subway/rail stations, airports, public buildings, and in transit systems. For example, the sensors may be utilized to monitor and detect deliberate release of harmful chemicals and explosives, thus protecting civilians from attacks. They may also be integrated into equipment carried or worn by soldiers for detection of harmful chemicals, explosives, or other terrorist elements.

Having generally described the invention, the same will be further understood through reference to the following additional examples, which are provided by way of illustration and are not intended to be limiting of the present invention unless specified.

### EXAMPLES

### Example 1

Nanowire-nanocluster hybrid chemical sensors were realized by functionalizing gallium nitride (GaN) nanowires (NWs) with titanium dioxide (TiO₂) nanoclusters for selectively sensing benzene and other related aromatic compounds.

### Materials and Methods

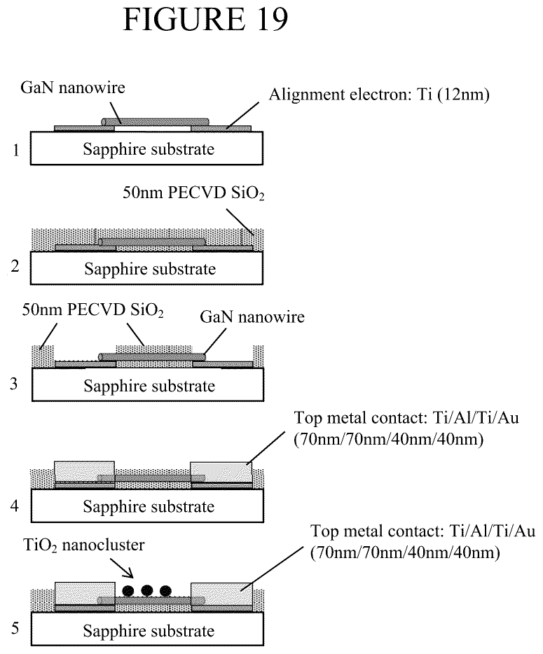

C-axis, n-type, Si-doped GaN grown by catalyst-free molecular beam epitaxy on Si (111) substrates were utilized. For details of NW growth, see Bertness K A et al. (2008) “Mechanism for spontaneous growth of GaN nanowires with molecular beam epitaxy,” J. Crystal Growth 310 (13): 3154-3158). An exemplary process of sensor fabrication is shown in FIG. 19. Post-growth device fabrication was done by dielectrophoretically aligning the nanowires on 9 mm×9 mm sapphire substrates (see Motayed A et al. (2006) “Realization of reliable GaN nanowire transistors utilizing dielectrophoretic alignment technique,” J. Appl. Phy. 100:114310). The device substrates had 12 nm thick Ti alignment electrodes of semi-circular geometry with gaps between them ranging from 4 μm to 8 μm. After the alignment of the nanowires, the samples were dried at 75° C. for 10 min on a hot plate for evaporation of the residual solvent. This was followed by a plasma enhanced chemical vapor deposition (PECVD) of 50 nm of SiO₂, at a deposition temperature of 300° C. This passivation layer was deposited to ensure higher yield for the fabrication process.

After the oxide deposition, photolithography was performed to define openings for the top contact. The oxide in the openings was etched using reactive ion etching (RIE) with CF₄/CHF₃/O₂ (50 sccm/25 sccm/5 sccm) gas chemistry. The top contact metallization was deposited in an electron-beam evaporator with base pressure of 10⁻⁵ Pa. The deposition sequence was Ti (70 nm)/Al (70 nm)/Ti (40 nm)/Au (40 nm). The oxide layer over the nanowires between the end contacts was then etched in buffered HF etching solution for 15 seconds. A negative resist was used to protect the end metal contacts from the etching solution.

The TiO₂ nanoclusters were deposited on the exposed GaN NWs using RF magnetron sputtering. The deposition was done at 325° C. with 50 sccm of Ar flow, and 300 W RF power. The deposition rate was about 0.2 Å/s. Thermal annealing of the complete sensor devices (GaN NW with TiO₂ nanoclusters) was done at 650° C. to 700° C. for 30 seconds in a rapid thermal processing system with 6 slpm (standard liter per min) flow of ultrahigh purity Ar. A relatively slow ramp rate of 100° C. per min was chosen to reduce the stress in the metal-nanowire contact area during heating. The anneal step was optimized to facilitate Ohmic contact formation to the GaN NWs and also to induce crystallization of the TiO₂ clusters. Additional lithography was performed to form thick metal bond pads with Ti (40 nm) and Au (160 nm).

The crystallinity and phase analysis of the sputtered TiO₂ films were assessed by X-ray diffraction (XRD). The XRD scans were collected on a Bruker-AXS D8 scanning X-ray micro-diffractometer equipped with a general area detector diffraction system (GADDS) using Cu-Kα radiation. The two-dimensional 2θ-χ patterns were collected in the 2θ=23° to 51° range followed by integration into conventional Ω-θ scans. The microstructure and morphology of the sputtered TiO₂ films used for fabrication of sensors were characterized by high-resolution analytical transmission and scanning transmission electron microscopy (HRTEM/STEM) and cold field-emission scanning electron microscopy (FESEM). GaN nanowires with sputtered TiO₂ were deposited onto a lacey carbon films supported by Cu-mesh grids and analyzed in a 300 kV TEM/STEM microscope. The instrument was equipped with an X-ray energy dispersive spectrometer (XEDS) and an electron energy-loss spectrometer (EELS) as well as bright-field (BF) and annular dark-field (ADF) STEM detectors to perform spot and line profile analyses.

The device substrates, i.e., the sensor chips, were wire-bonded on a 24 pin ceramic package for the gas sensing measurements. The device characterization and the time dependent sensing measurements were done using an Agilent B1500A semiconductor parameter analyzer. Each sensor chip was placed in a custom-designed stainless steel test chamber of volume 0.73 cm³ with separate gas inlet and outlet. The test chamber had a quartz window on top for UV excitation provided by a 25 W deuterium bulb (DH-2000-BAL, Ocean Optics) connected to a 600 μm diameter optical fiber cable with a collimating lens at the end for uniform illumination over the sample surface. The operating wavelength range of the bulb was 215 to 400 nm. The intensity at 365 nm measured using an optical power meter was 375 nW cm⁻². For all the sensing experiments regular breathing air (<9 ppm of water) was used as the carrier gas. A wide range of concentrations from 1% to as low as 50 parts per billion (ppb) of various organic compounds were achieved with a specific arrangement of bubbler and mass flow controllers (MFCs). During the sensor measurements, the net flow (air+VOC mix) into the test chamber was set to a constant value of 20 sccm. After the sensor devices were exposed to the organic compounds, they were allowed to regain their baseline current with the air-chemical mixture turned-off, without purging or evacuating the test-chamber.

### Results

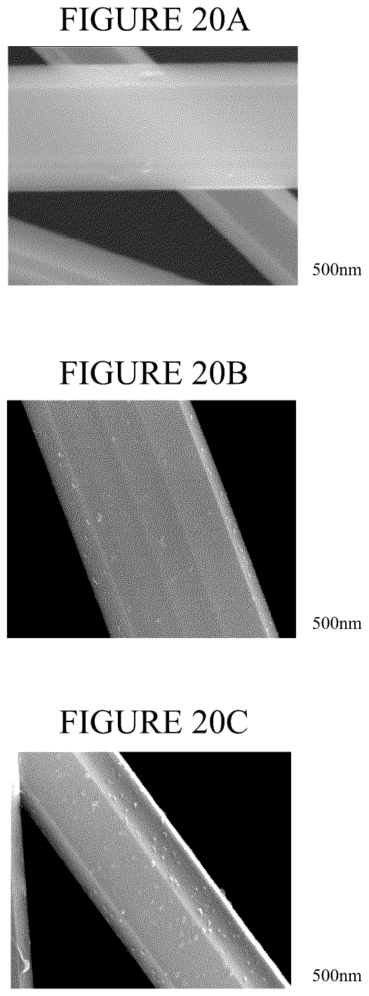

FIG. 20 shows GaN nanowires with three different nominal thicknesses of TiO₂ coatings sputtered on them: 2 nm (FIG. 20, plate (a)); 5 nm (FIG. 20, plate (b)); and 8 nm (FIG. 20, plate (c)). Rather sparse, well-defined clusters can be seen for both the 5 nm and 8 nm area-averaged sputtered coatings of TiO₂. The average size of these large clusters was about 15 nm. For the 8 nm sputtered coating, the coverage of the TiO₂ clusters is much denser. However, TEM studies revealed the presence of clusters with much smaller diameter (less than about 4 nm) on the nanowire surface.

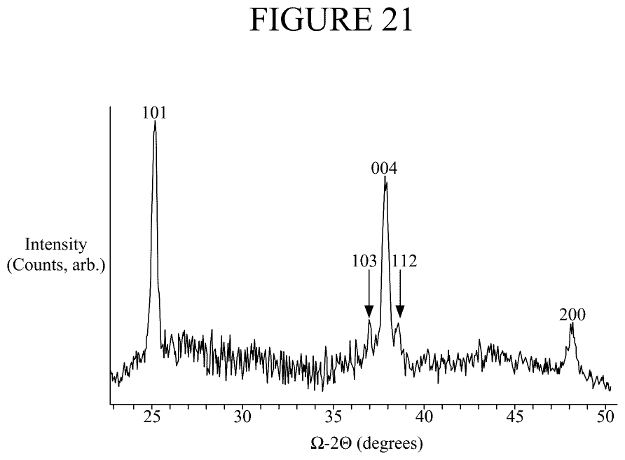

Detection of XRD signal from the TiO₂ decorated GaN NWs was difficult due to the minuscule size and total volume of TiO₂ nanoclusters. We therefore prepared a 150 nm thick TiO₂ film by sputtering it onto a SiO₂ coated Si substrate at 300° C. followed by anneal at 650° C. for 45 s in argon. The processing conditions produced an identical morphology as in the TiO₂ decorated NW case. Referring to FIG. 21, we identified from the XRD that TiO₂ is in the single-phase anatase form. As-deposited TiO₂ films were found to be amorphous.

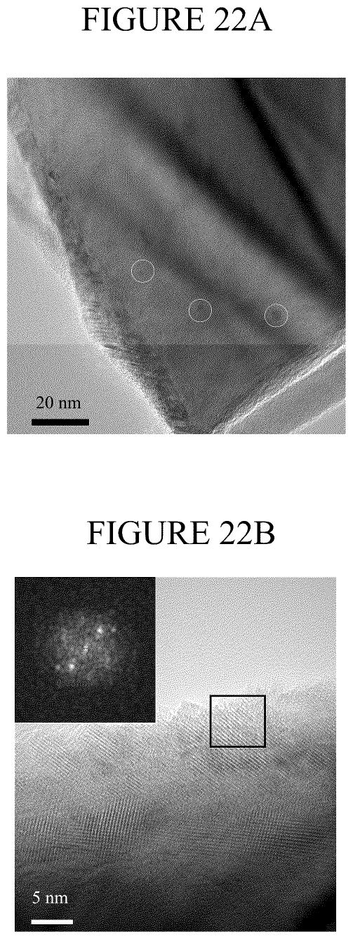

The XRD results agree with the TEM analysis on TiO₂ decorated GaN NWs, which revealed that upon annealing at 700° C. for 30 s, the TiO₂ islands became partially crystalline, as shown in FIG. 22. Three most common phases of TiO₂ are anatase, rutile, and brookite. Thermodynamic calculations predict that rutile is the most stable TiO₂ phase in the bulk state at all temperatures and atmospheric pressure (see Norotsky A et al. (1967) “Enthalpy of Transformation of a High-Pressure Polymorph of Titanium Dioxide to the Rutile Modification,” Science 158:338; Jamieson J C and Olinger B (1969) “Pressure-temperature studies of anatase, brookite, rutile, and TiO₂ (II); A discussion,” Am. Min. 54:1477-1480). However, comparative experiments with particle size showed that the phase stability might reverse with decreasing particle size, possibly due to the influence of surface free energy and surface stress (Zhang H Z and Banfield J. F (2000) “Understanding polymorphic phase transformation behavior during growth of nanocrystalline aggregates: insights from TiO₂,” J. Phys. Chem. B 104:3481-3487). Anatase is the most stable phase when the particle size is less than about 11 nm, whereas rutile is most stable at sizes greater than about 35 nm. Although both rutile and anatase TiO₂ are commonly used as photocatalyst, anatase form shows greater photocatalytic activity for most reactions (Linsbigler A L et al. (1995) “Photocatalysis on TiO₂ Surfaces: Principles, Mechanisms, and Selected Results,” Chem. Rev. 95:735-7; Tanaka K et al. (1991) “Effect of crystallinity of TiO₂ on its photocatalytic action,” Chem. Phys. Lett. 187:73-76). This is one consideration for sputtering nominally 8 nm of TiO₂ for the sensor fabrication.

Although we have sputtered 8 nm of TiO₂ for fabricating the hybrid sensors, for the TEM studies 20 nm of TiO₂ coating was utilized. The thick GaN nanowires prevented acquisition of any TEM diffraction from thinner TiO₂ coatings. The TEM results presented for 20 nm thick TiO₂ was representative of the clusters formed for 8 nm deposited TiO₂ in actual sensors. Typical morphologies of a 20 nm thick TiO₂ film sputtered on n-GaN nanowires and annealed at 700° C. for 30 seconds are illustrated by TEM data in FIG. 22. The TEM image in FIG. 22, plate (a) shows 2 nm to 10 nm diameter individual TiO₂ particles non-uniformly distributed on the surface of a GaN nanowire. Some of the particles are identified by circles. Crystallinity of some nanoparticles observed is shown in the HRTEM image in FIG. 22, plate (b) with nanocrystallites on the edge of a GaN nanowire with the sputtered TiO₂. The FFT pattern from the boxed area is seen in exploded view in the upper left inset image, showing 0.35 nm lattice fringes which are consistent with a (101) reflecting plane of anatase but not available in hexagonal wurtzite-type GaN crystals.

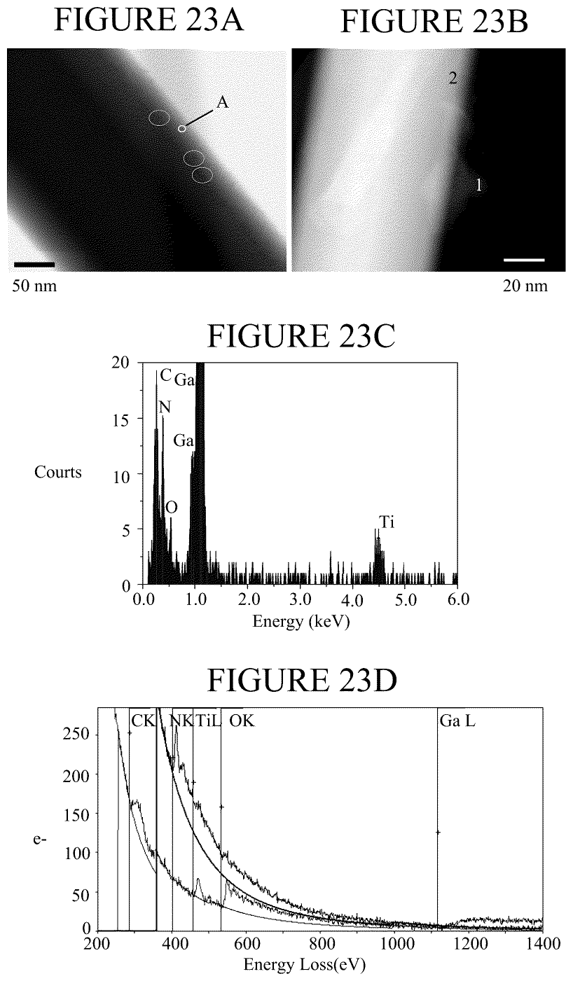

Referring to FIG. 23, plate (a), a BF-STEM image shows 5 to 10 nm TiO₂ nanoparticles barely visible against the GaN nanowire. An ADF-STEM image of a TiO₂ island on a GaN nanowire is shown in FIG. 23, plate (b). The presence of TiO₂ was confirmed by analysis of selected areas as well as of individual particles using XEDS and EELS and nanoprobe capabilities. Referring to FIG. 23, plate (c), the X-ray spectrum of an individual 5 nm TiO₂ particle (identified by the marked circle “A” in FIG. 23, plate (a)) exhibits the Tikα peak at 4.51 keV and the weak Okα peak at 0.523 keV. The NKα peak at 0.39 keV and gallium lines (the GaL series at 1.0 keV to 1.2 keV) and the CKα peak at 0.28 keV are also identified. EEL spectrum acquired at Position “1” marked in FIG. 23, plate (b) (the tip of a TiO₂-containing aggregate) exhibits the TiL edge at 456 eV and the OK edge at 532 eV and also the CK edge at 284 eV. A reference spectrum recorded at Position 2 marked in FIG. 23, plate (b) (an edge of the GaN nanowire) reveals traces of titanium and oxygen with the NK edge at 401 eV and the Gal edge at 1115 eV, respectively.

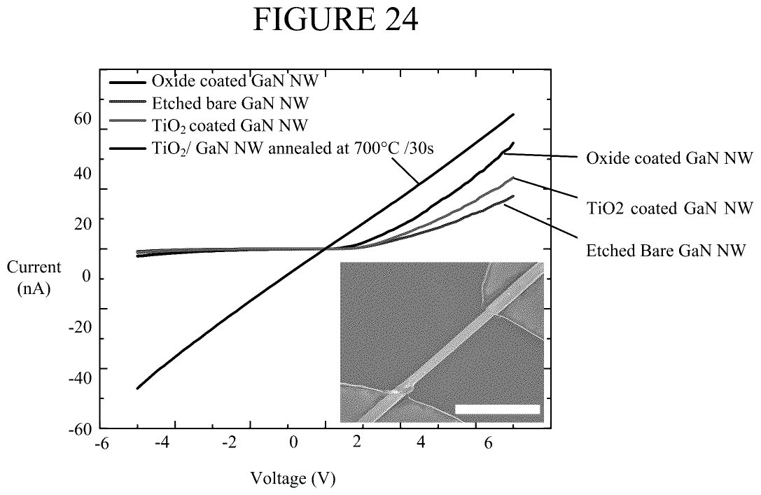

FIG. 24 shows the current-voltage (I-V) characteristics of a GaN NW two-terminal device at different stages of processing. The I-V curves of the as-deposited devices were non-linear and asymmetric. The current decreased when the SiO₂ layer over the NW was etched. However, the current increased with the deposition of TiO₂ nanoclusters. Oxygen adsorption on the bare GaN nanowire surface can introduce surface states (Zywietz et al. (1999) “The adsorption of oxygen at GaN surfaces,” Appl. Phys. Lett. 74:1695), resulting in the decrease of the nanowire conductivity. The devices annealed at 700° C. for 30 seconds showed significant changes in their I-V characteristics with a majority of the devices exhibiting linear I-V curves. This is consistent with the fact that low resistance ohmic contacts to the nitrides require annealing at 700° C. to 800° C. (see Motayed A et al. (2003) “Electrical, thermal, and microstructural characteristics of Ti/Al/Ti/Au multilayer ohmic contacts to n-type GaN,” J. Appl. Phys. 93 (2): 1087-1094).

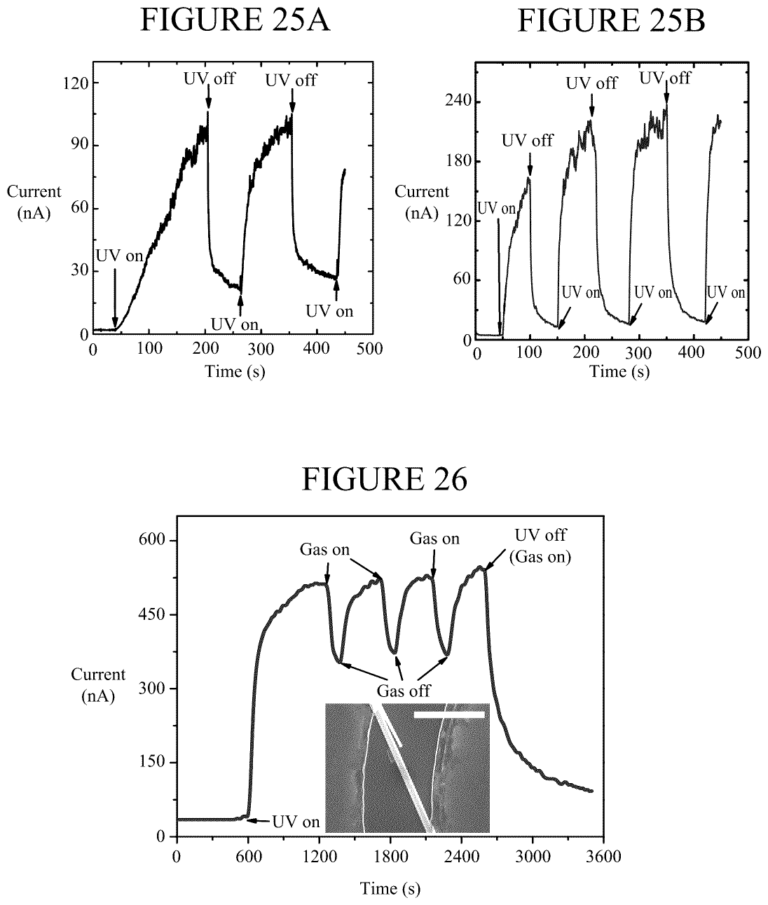

FIG. 25 shows the photoconductance of a bare GaN NW device and the TiO₂ coated GaN NW device. The NW devices with TiO₂ nanoclusters showed almost two orders of magnitude increase in the current when exposed to UV light as compared to the similar diameter bare NW devices. Increase of photoconductance due to surface functionalization has been observed in ZnO nanobelts coated with different polymers (Lao C S et al. (2007) “Giant Enhancement in UV Response of ZnO Nanobelts by Polymer Surface-Functionalization,” J. Am. Chem. Soc. 129:12096-12097). This enhancement of photoconductance is often attributed to the separation of photogenerated charge carriers by a surface depletion field, thereby increasing the lifetime of the photogenerated carriers. After the light is turned off, the photo current decays rapidly, but not to the dark current value, which is likely due to the persistent photoconductivity of the NWs (see Sanford N A et al. (2010) “Steady-state and transient photoconductivity in c-axis GaN nanowires grown by nitrogen-plasma-assisted molecular beam epitaxy,” J. Appl. Phy. 107:034318).

The current through the bare GaN NW devices did not change when exposed to different VOCs mixed in air, even for concentrations as high as few percents. On the other hand, the TiO₂ coated hybrid devices responded even to the pulses of 20 sccm airflow. This is expected, considering that the conduction in most metal-oxides is affected by the presence of oxygen. The response of the TiO₂ nanocluster-GaN nanowire hybrid sensor to 1000 ppm of toluene in air is illustrated in FIG. 26. Exposure to the VOC in the dark had no effect on the hybrid device. However, in presence of UV excitation, when 1000 ppm of toluene (mixed in air) was introduced into the gas chamber, the sensor photocurrent decreased dramatically to approximately ⅔ of its base value. After 100 seconds of gas exposure, the gas flow is turned off and the sensor is allowed to recover at room temperature without any additional purging. The repeatability of the sensing action of these hybrid sensors is evident from FIG. 26.

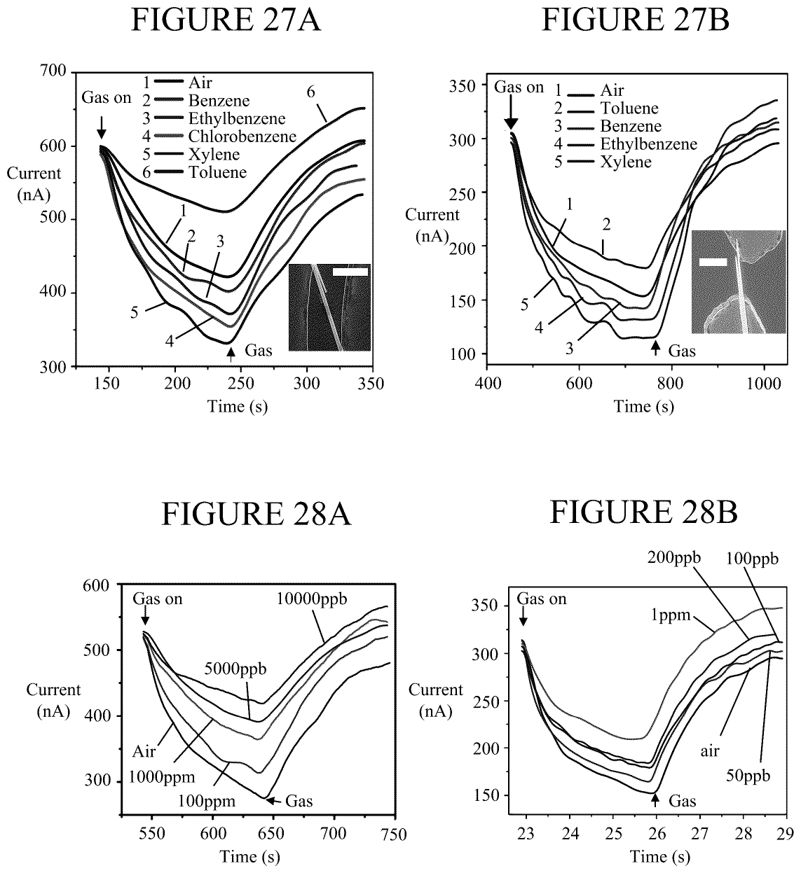

Interestingly, the hybrid sensors did not respond when exposed to methanol, ethanol, isopropanol, chloroform, acetone, and 1,3-hexadiene, even for concentrations as high as several percent. Also, the photocurrent for these sensors increased with respect to air when exposed to toluene vapors, whereas for every other aromatic compound, the photocurrent decreased relative to air, as shown in FIG. 27, plate (a). More than twenty sensor devices were tested, with all exhibiting the same trend. In addition, the use of toluene from different sources resulting in the same sensor behavior. FIG. 27, plate (b) shows the response of a different device for 200 ppb concentrations of the same chemicals. It is clear that even for toluene concentration as low as 200 ppb, the relative change in photocurrent is the reverse of that observed with other chemicals. If the photocurrent in the presence of air for these sensors is used as their baseline calibration, then we can distinctly identify toluene from other aromatic compounds present in air using our hybrid devices. The response time is defined as the time taken by the sensor current to reach 90% of the response (I_{f}-I₀) when exposed to the analyte. The If is the steady sensor current level in the presence of the analyte, and I₀ is the current level without the analyte, which in our case is in the presence of air. The recovery time is the time required for the sensor current to recover to 30% of the response (I_{f}-I₀) after the gas flow is turned off (Garzella C et al. (2000) “TiO₂ thin films by a novel sol-gel processing for gas sensor applications,” Sens. and Actuators B: Chemical 68:189-196). The response and recovery times for ppm levels of BTEX concentrations were ≈60 seconds and ≈75 seconds, respectively. The response and recovery times for ppb levels of concentrations were =180 seconds and ≈150 seconds, respectively. In contrast, conventional nanowire/nanotube sensors reported in the literature as working at room-temperatures had much longer response times in minutes (Leghrib R et al. (2010) “Gas sensors based on multiwall carbon nanotubes decorated with tin oxide nanoclusters,” Sens. and Actuators B: Chemical 145:411-416; Balazsi C et al. (2008) “Novel hexagonal WO3 nanopowder with metal decorated carbon nanotubes as NO2 gas sensor,” Sensors and Actuators B: Chemical 133:151-155; Kuang Q et al. (2008) “Enhancing the photon-and gas-sensing properties of a single SnO2 nanowire based nanodevice by nanoparticle surface functionalization,” J. Phys. Chem. C 112:11539-11544; Lim W et al. (2008) “Room temperature hydrogen detection using Pd-coated GaN nanowires,” Appl. Phys. Lett. 93:072109). Fast response and recovery times indicate fast adsorption and desorption, which is attributed to the enhanced reactivity of the nanoscale TiO₂ clusters.

The responses of two hybrid devices to different concentrations of toluene in air are shown in FIG. 28. FIG. 28, plate (a) shows the change of photocurrent of a 234 nm diameter device when exposed to toluene concentrations from 10000 ppm down to 100 ppm. FIG. 28, plate (b) shows the photocurrent of a sensor device with 170 nm diameter wire for toluene concentrations from 1 ppm to 50 ppb.

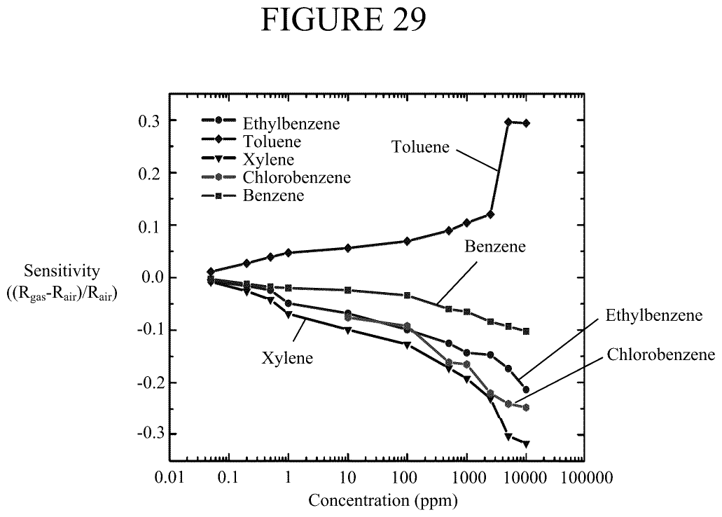

Sensitivity is defined as (R_{gas}−Rₐᵢᵣ)/Rₐᵢᵣ, where R_{gas}, Rₐᵢᵣ are the resistances of the sensor in the presence of the chemical-air mixture and in the presence of air, respectively. The sensitivity plots of a hybrid device for different VOCs tested are shown in FIG. 29. The sensitivity plot emphasizes the ability of these hybrid sensors to reliably detect BTEX (benzene, toluene, ethylbenzene, chlorobenzene, and xylene), which are common indoor and outdoor pollutants with wide detection range (50 ppb to 1%).

### Example 2

The sensing behavior of three NWNC based hybrid sensors was compared: 1) GaN NW coated with TiO₂ NCs (hereafter referred to as GaN/TiO₂ NWNC hybrids); 2) GaN NW coated with TiO₂ and Pt multicomponent NCs (i.e., GaN/(TiO₂—Pt) NWNC hybrids); and 3) GaN NW coated with Pt NCs (i.e., GaN/Pt NWNC hybrids). It was found that sensors with TiO₂—Pt multicomponent NCs on GaN NW were only sensitive to methanol, ethanol, and hydrogen. Higher carbon-containing alcohols (such as n-propanol, iso-propanol, n-butanol) did not produce any sensor response. These sensors had the highest sensitivity towards hydrogen. Prior to the Pt deposition, the GaN/TiO₂ NWNC hybrids did not exhibit any response to alcohols, however they detected benzene and related aromatic compounds such as toluene, ethylbenzene, xylene, and chlorobenzene mixed with air. The GaN/Pt hybrids only showed sensitivity to hydrogen and not to methanol or ethanol. The sensitivity of GaN/Pt hybrids towards hydrogen was lower compared to the GaN/(TiO₂—Pt) hybrids.

### Materials and Methods

GaN NWs utilized for this study were c-axis, n-type (Si-doped), grown by catalyst-free molecular beam epitaxy as described by Bertness K A et al. (2008), supra, J. Crystal Growth 310 (13): 3154-3158. Post-growth device fabrication was done by dielectrophoretically aligning the nanowires on 9 mm×9 mm sapphire substrates. The details of the device fabrication are set forth in Example 1. After fabrication of two-terminal GaN NW devices, the TiO₂ NCs were deposited on the GaN NW surface using RF magnetron sputtering. The deposition was done at 325° C. with 50 standard cubic centimeters per minute (sccm) of Ar flow, and 300 W RF power. The nominal deposition rate was about 0.24 Å/s. Thermal annealing of the complete sensor devices (GaN NW with TiO₂ nanoclusters) was done at 700° C. for 30 seconds in a rapid thermal processing system. For TiO₂—Pt composite NCs, the Pt was sputtered using DC sputtering after annealing of the TiO₂ clusters on GaN NW. The Pt sputtering was done with an Ar flow of 35 sccm, at a pressure of 1.3 Pa and power of 40 W for 10 seconds. For the Pt/GaN devices Pt was sputtered on bare GaN NWs after annealing the ohmic contacts at 700° C. for 30 seconds. Additional lithography was performed to form thick metal bond pads with Ti (40 nm) and Au (200 nm). The device substrates, i.e., the sensor chips, were wire-bonded on a 24 pin ceramic package for the gas sensing measurements.

The microstructure and morphology of the sputtered TiO₂ films used for the fabrication of the sensors were characterized by high-resolution transmission and scanning transmission electron microscopy (HRTEM/STEM), selected-area electron diffraction (SAED), and field-emission scanning electron microscopy (FESEM). For the TEM characterization, the GaN NWs were dispersed on 10 nm thick carbon films supported by Mo-mesh grids, followed by the deposition of TiO₂ NCs and annealing, and subsequent Pt deposition. The samples were analyzed in a FEI Titan 80-300 TEM/STEM microscope operating at 300 kV accelerating voltage and equipped with S-TWIN objective lenses, which provided 0.13 nm (STEM) and 0.19 nm (TEM) resolution by points. The instrument also had a Gatan CCD image acquisition camera, bright-field (BF), ADF and high-angle annular dark-field (HAADF) STEM detectors to perform spot, line profile, and areal compositional analyses using an EDAX 300 kV high-performance Si/Li X-ray energy dispersive spectrometer (XEDS).

The as-fabricated sensors were placed in a custom designed gas chamber for gas exposure measurements. The device characterization and the time dependent sensing measurements were done using an Agilent B1500A semiconductor parameter analyzer. The gas sensing experiments have been performed by measuring the electrical conductance of the devices upon exposure to controlled flow of air/chemical mixture in presence of UV excitation (25 W deuterium bulb operating in the 215 nm to 400 nm range). For all the sensing experiments with chemicals, breathing air (<9 μmol/mol of water) was used as the carrier gas. For the hydrogen sensing we used high-purity nitrogen as the carrier gas. After the sensor devices were exposed to the organic compounds and hydrogen, they were allowed to regain their baseline current with the air-chemical mixture turned-off, without purging or evacuating the test-chamber.

### Results

### Morphological and Structural Characterization of NWNC Hybrids

It was challenging to measure the sizes and shapes of small TiO₂ and Pt particles on the surfaces GaN NWs from greyscale TEM images due to: a) 270 nm to 300 nm thickness of the NWs used in the devices and variations of thickness and curvature across the structure; b) diffraction contrast induced particularly by bending of the wires-even similar particles could appear as having different intensities, while local thickness variations of the carbon support film could result in variable contrast affecting the mean intensity values of the particles; c) overwhelming domination of electron diffraction in SAED from the GaN NW over the diffraction from TiO₂ and Pt nanoparticles. To overcome these problems, TEM imaging was conducted under minimal beam intensity conditions close to the Scherzer defocus at highest available accelerating voltage of 300 kV using both stationary beam (bright-field TEM/SAED, phase-contrast high-resolution TEM) and scanning beam (STEM/XEDS) modes. Areas for analyses were selected near the wire's edges and on the amorphous carbon support film in the vicinity of the NWs.

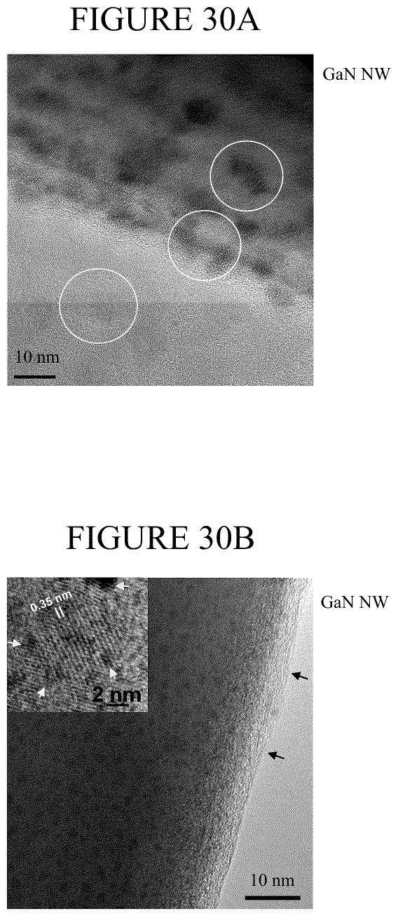

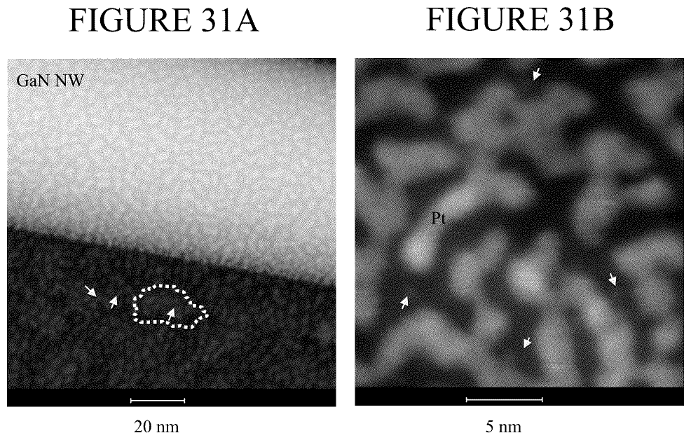

FIG. 30 shows HRTEM micrographs of a GaN NW on a thin amorphous carbon support films with TiO₂ coating, before and after the Pt deposition. The deposited TiO₂ layer formed an island-like film, where 10 nm to 50 nm partially aggregated particles (see FIG. 30, plate (a)) were often interconnected into extended two-dimensional networks. This was consistent with SAED and compositional analyses of deposited TiO₂ films indicating a mixture of polycrystalline anatase and rutile and of the same mixture plus fcc Pt nanoparticles (FIG. 30, plate (b)), respectively. Pt crystalline particles with 1 to 5 nm size were randomly distributed on the surfaces of TiO₂ islands and sometimes were partially coalesced forming elongated aggregates. In the latter case, significant thickness of the GaN NWs made it difficult to visualize TiO₂ deposits due to the limited contrast difference between TiO₂ and GaN and presence of multiple heavy Pt particles. In spite of these limitations, detailed HRTEM and HR-STEM observations revealed 0.35 nm (101) hcp lattice fringes belonging to anatase (see FIG. 30, plate (b), upper left inset) and 0.23 nm to 0.25 nm (111) and 0.20 nm to 0.22 nm (200) fcc lattice fringes belonging to Pt nanocrystallites, respectively, as well as amorphous-like Pt clusters with diameters around 1 nm or less (see FIG. 31, plates (a) and (b)).

In the FIG. 31, HAADF-STEM image shows 1 nm to 5 nm diameter bright Pt nanoparticles and barely visible TiO₂ islands (medium grey) randomly distributed near the edge of the nanowire. The presence of both TiO₂ and Pt nanocrystallites was confirmed by the analysis of selected areas using XEDS nanoprobe capabilities.

### Current-Voltage (I-V) Characteristics of NWNC Hybrids in Dark

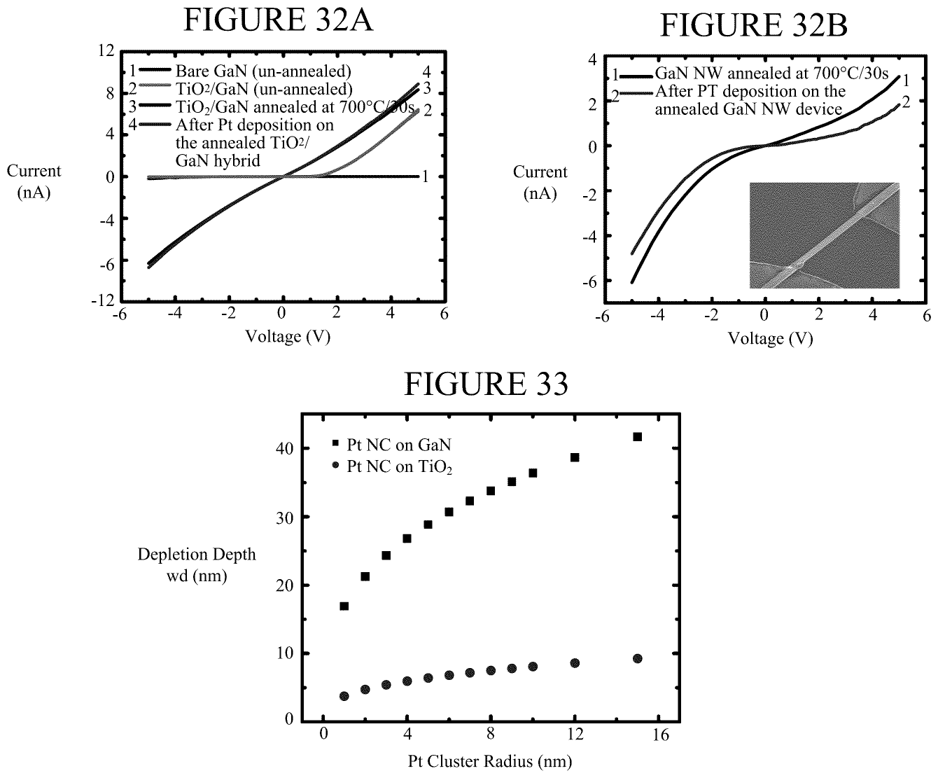

FIG. 32 shows the I-V characteristics of the GaN/(TiO₂—Pt) and GaN/Pt hybrid sensor devices at different stages of processing. A plan-view SEM image of an exemplary sensor device is shown in the inset of FIG. 32, plate (b) for representation purposes. The I-V curves of the as-fabricated GaN NW two-terminal devices were non-linear and asymmetric. A small increase in the positive current after the deposition of TiO₂ nanoclusters (curve 2) can be attributed to decreased surface depletion of the GaN NW due to passivation of surface states, and/or the high temperature deposition (325° C.) of the nanoclusters initiating ohmic contact formation. The devices annealed at 700° C. for 30 s after the deposition of TiO₂ NCs showed significant change in their I-V characteristics with a majority of the devices exhibiting linear I-V curves. Interestingly, Pt NC deposition on TiO₂ coated GaN NWs further increased the conductivity of the nanowire. This is due to the fact that the Pt clusters depleted the TiO₂ clusters by removing free electrons. Increased depletion in the TiO₂ clusters due to Pt would decrease TiO₂ induced depletion in the GaN NW, leading to an increase in the NW current. With the Pt/GaN hybrids, the current decreases followed by the deposition of Pt (see FIG. 32, plate (b)) as expected due to the depletion region formed in the NW under the metal clusters.

The nature of the depletion region formed by the nano-sized metal clusters on a semiconductor may be determined by Zhdanov's model. FIG. 33 shows the calculated zero-bias depletion depth produced in GaN and TiO₂ respectively, as a function of the Pt cluster radius according to Equation (1). For calculating the depletion depth we assumed the effective conduction band density of states in TiO₂ as 3.0×10²¹ cm⁻³ and point-defect related donor concentration as 1.0×10¹⁸ cm⁻³ [43,44]. The electron concentration in the GaN NWs was measured to be 1×10¹⁷ cm⁻³ in a separate experiment.

FIG. 33 indicates that even a single Pt NC of 2 nm radius can significantly deplete a 10 nm (average size) TiO₂ cluster. The effect of TiO₂ depletion on GaN NW is difficult to determine as it could be influenced by numerous factors including interface states and particle geometry. Given the very high density of TiO₂ clusters on the NW surface (see FIG. 31, plate (b)), it is clear that the Pt particles mostly reside on the surfaces of TiO₂ NCs. However, from FIG. 33 we can see that when Pt NCs are directly on GaN, they deplete the carriers in an even larger region in the GaN NW. This qualitatively explains the relatively larger change in current observed when Pt NCs were deposited on bare GaN NWs as compared to the change in current when Pt NC were deposit on the TiO₂-coated NWs.

Comparative Sensing Behavior of GaN/(TiO₂—Pt), GaN/Pt and GaN/TiO₂ NWNC Hybrid Sensors

The photocurrent through the bare GaN NW devices did not change when exposed to different chemicals mixed in air, even for concentrations as high as 3%. In contrast, the TiO₂-coated hybrid devices responded even to the pulses of 20 sccm airflow in the presence of UV excitation. The response of the TiO₂ NC-coated GaN nanowire hybrid sensors to different concentrations of benzene, toluene, ethylbenzene, chlorobenzene, and xylene in air is discussed above. The GaN/TiO₂ hybrids showed no response when exposed to other chemicals such as alcohols, ketones, amides, alkanes, nitro/halo-alkanes, and esters.

Remarkably, after the deposition of Pt nanoclusters on the GaN/TiO₂ hybrids, the sensors were no longer sensitive to benzene and other aromatic compounds, but responded only to hydrogen, methanol, and ethanol. In addition, the GaN/(TiO₂—Pt) hybrids showed no response when exposed to higher carbon-containing (C>2) alcohols such as n-propanol, iso-propanol, and n-butanol. FIG. 5 shows the change of photocurrent of a GaN/(TiO₂—Pt) sensor in the presence of 20 sccm air flow of air mixed with 1000 μmol/mol (ppm) of methanol, ethanol, and water, respectively, and 20 sccm of nitrogen flow mixed with 1000 μmol/mol (ppm) hydrogen. The change in the photocurrent of the sensor when 20 sccm of breathing air is flowing through the test chamber serves as a reference for calculating the sensitivity of the sensors. The sensitivity is defined as (R_{gas}−Rₐᵢᵣ)/Rₐᵢᵣ, where R_{gas} and Rₐᵢᵣ are the resistances of the sensor in the presence of the analyte-air mixture and in the presence of air only, respectively (Rₐᵢᵣ is replaced with R_{nitrogen} for hydrogen sensing experiments).

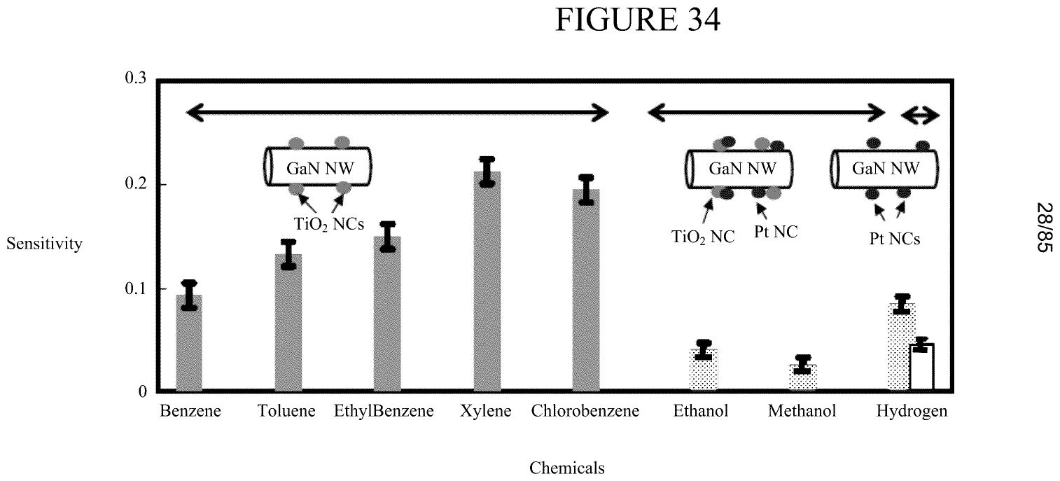

The GaN/TiO₂ hybrids without Pt showed no response to hydrogen and the alcohols. Interestingly, when Pt NC-coated GaN NW hybrids (GaN/Pt) with the same nominal thickness were tested, they showed very limited sensitivity only to hydrogen and not to any alcohols. The comparative summary of the sensing behavior of the three different hybrids are presented in FIG. 34.

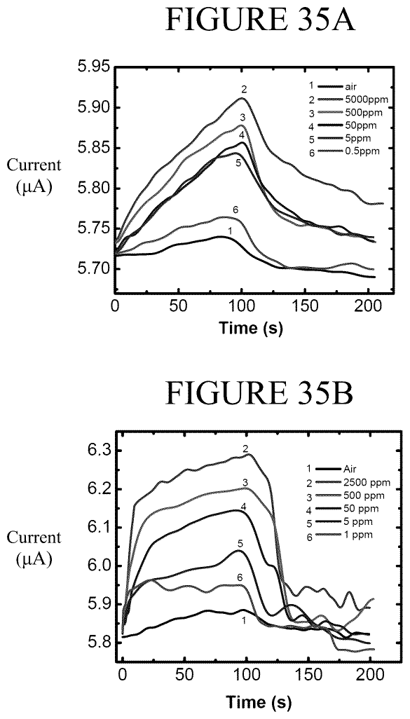

The response of the GaN/(TiO₂—Pt) NWNC sensor to different concentrations of methanol in air is shown in FIG. 35, plate (a). FIG. 35, plate (b) shows the response to different concentrations of hydrogen in nitrogen for the same GaN/(TiO₂—Pt) NWNC sensor device. The sensor response is much higher for hydrogen compared to methanol and ethanol. The response time is also much shorter for hydrogen as compared to methanol, and the sensor photocurrent saturates after initial 20 s exposure.

The response time was defined as the time taken by the sensor current to reach 90% of the response (I_{f}−I₀) when exposed to the analyte. The If is the steady sensor current level in the presence of the analyte, and I₀ is the current level without the analyte, which in our case is in the presence of 20 sccm of air flow. The recovery time is the time required for the sensor current to recover to 30% of the response (I_{f}−I₀) after the gas flow is turned off (see Garzella C et al. (2000) Sensors and Actuators B: Chemical 68:189-196). The response time for hydrogen was ≈60 seconds, whereas the response time for ethanol and methanol was ≈80 seconds. The sensor recovery time for hydrogen was ≈45 seconds and the recovery times for ethanol, methanol was ≈60 seconds and ≈80 seconds, respectively. For comparison, Wang et al. demonstrated a conventional ZnO NW-based hydrogen sensor with a response time of 10 minutes for 4.2% sensitivity (Wang H T et al. (2005) “Hydrogen-selective sensing at room temperature with ZnO nanorods,” Appl. Phys. Lett. 86:243503).

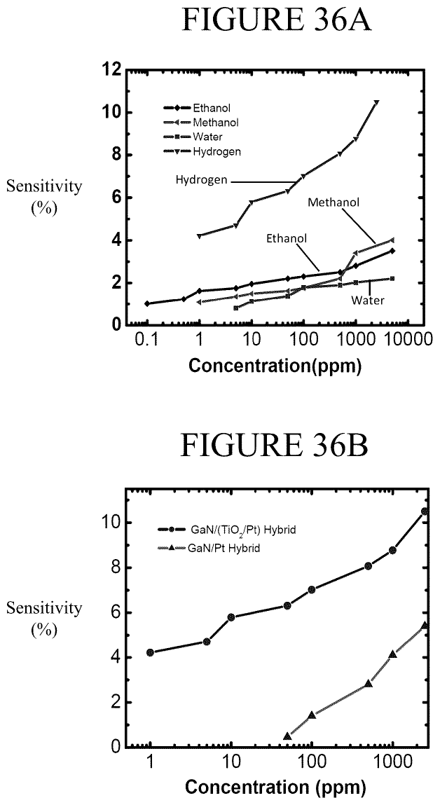

The sensitivity plot of a GaN/(TiO₂—Pt) hybrid device for the various analytes tested is shown in FIG. 36, plate (a). Note that the lowest concentration detected for methanol and hydrogen (1 ppm or μmol/mol) is not the sensor's detection limit, but a system limitation. It can be seen that the sensor is more sensitive to methanol than ethanol for concentrations ≥1000 μmol/mol (ppm), and the relative sensitivity switches for concentrations of 500 μmol/mol (ppm) and below. Similar behavior is observed with twenty unique devices, possibly due to difference in surface coverage of the different alcohols over the concentration range. FIG. 36, plate (b) is a comparative plot showing the sensitivity of GaN/(TiO₂—Pt) and GaN/Pt hybrid sensors to hydrogen in nitrogen. The GaN/Pt hybrid devices showed relatively low sensitivity with detection limit of 50 μmol/mol (ppm), below which the devices stopped responding. The gas exposure time was also increased to 200 seconds for the GaN/Pt devices to obtain increased response compared to 100 seconds for the GaN/(TiO₂—Pt) GaN devices. The sensitivity of the GaN/(TiO₂—Pt) sensors was greater for alcohols and hydrogen when compared with the same concentrations of water in air, which thus enables their use in high-humidity conditions.

Table X and Table XI compare the performance of the sensor devices of the present invention with sensors disclosed in the most recent literature in terms of operation temperature, carrier gas, lower detection limit, and response/recovery times. The comparison indicates that the sensors devices of the present invention exhibit an excellent response to very low concentrations of analytes (100 ppb for ethanol and 1 ppm for hydrogen) at room temperature, with air as the carrier gas. The testing conditions closely resembled real-life conditions, which underlines the significance of the disclosed sensors. The response and recovery times were also lower for the disclosed sensors compared to the other conventional sensors, as shown in Tables X and XI.

TABLE X
Performance of GaN/(TiO₂—Pt) NWNC hybrid sensors
to ethanol in comparison with conventional sensors

| | Response/ | Lower | | | |
|---|---|---|---|---|---|
| | Recovery | Detection | | | Testing |
| | Time | Limit | | Carrier Gas | Temperature |
| Sensor of | 80 s/75 s | 100 ppb with | | air | Room |
| Present | | 1% | | | temperature (RT) |
| Invention | | Sensitivity⁴ | | | |
| CNT¹/SnO₂ core | 1 s/10 s | 10 | ppm | air | 300° C. |
| shell | | | | | |
| nanostructures | | | | | |
| MWCNTs²/ | 20 s/20 s | 18,000 | ppm | air | RT |
| NaClO₄/ | | | | | |
| polypyrrole | | | | | |
| Metal-CNT | ~2 min/ | 500 ppb with | | N₂ in a | RT |
| hybrids | (recovery | sensitivity <1% | | vacuum test | |
| | time not | | | chamber | |
| | reported) | | | | |
| V₂O₅ nanobelts | 50 s/50 s | 5 | ppm | air | 150° C.-400° C. |
| ZnO nanorods | 3.95 min/ | 10 | ppm | Synthetic air | 125° C.-300° C. |
| | 5.3 min | | | | |
| ZnO nanowires | 10 s/55 s | 1 | ppm | air | 220° C. |
| ITO³ nanowires | 2 s/2 s | 10 | ppm | air | 400° C. |
| SnO₂ nanowires | 2 s/2 s | 10 | ppm | air | 300° C. |

¹Carbon nanotubes
²Multiwall carbon nanotubes
³Indium tin oxide
⁴Sensitivity values for sensors with lowest limit similar to disclosed results were compared.

TABLE XI
Performance of GaN/(TiO₂—Pt) NWNC hybrid sensors
to hydrogen in comparison with conventional sensors

| | | | | Testing |
|---|---|---|---|---|
| | Response/ | | | Temper- |
| | recovery times | Lower detection limit | | ature |
| Sensor of Present | 60 s/45 s | 1 ppm with sensitivity | | RT |
| Invention | | of 4% | | |
| CNT films | 5 min/30 s | 10 | ppm | RT |
| SWCNT/SnO₂ | 2 s/2 s | 300 | ppm | 250° C. |
| Pd/CNTs | 5 min/5 min | 30 ppm with sensitivity | | RT |
| | | of 3% | | |
| Pd/Si NWs | 1 hr/50 min | 3 | ppm | RT |
| Pt doped SnO₂ | 2 min/10 min | 100 | ppm | 100° C. |

NWs

The present results indicate the unique ability to tailor the selectivity of NWNC chemical sensors. With infinite combinations of metal and metal-oxide composite clusters available, there is a huge potential for sensor designs targeted for a multitude of applications.

### Example 3

Alcohol sensors using gallium nitride (GaN) nanowires (NWs) functionalized with zinc oxide (ZnO) nanoparticles are demonstrated. These sensors operate at room temperature, are fully recoverable, and demonstrate a response and recovery time on the order of 100 seconds. The sensing is assisted by UV light within the 215-400-nm band and with the intensity of 375 nW/cm² measured at 365 nm. The ability to functionalize an inactive NW surface, with analyte-specific active metal-oxide nanoparticles, makes this sensor suitable for fabricating multianalyte sensor arrays.

### Methods and Materials

Si-doped c-axis n-type GaN NWs were grown using catalyst-free molecular beam epitaxy on Si (III) substrate as described in Bertness K A et al. (2008), supra, J. Cryst. Growth 310 (13): 3154-3158. The NW diameter and length were in the ranges of 250-350 nm and 21-23 μm, respectively. The GaN NWs were detached from the substrate by sonication in isopropanol and dielectrophoretically aligned across the pre-patterned electrodes. The electrodes were fabricated using photolithography followed by deposition of a metal stack of Ti (40 nm)/Al (420 nm)/Ti (40 nm). Thick bottom electrodes ensure the free suspension of the NWs. For the formation of ohmic contacts to the NW ends, the top metal contacts were fabricated using a metal stack of Ti (70 nm)/Al (70 nm)/Ti (40 nm)/Au (40 nm), as described in A. Motayed et al. (2003), supra, J. Appl. Phys. 93 (2): 1087-1094. Rapid thermal anneal (RTA) was performed at 700° C. for 30 seconds in argon atmosphere to promote the formation of ohmic contacts and to reduce the stress in the thick bottom electrodes. Finally, ZnO nanoparticles were sputter deposited on the NW device with an RF power of 300 W in 60 standard cubic centimeters per minute (sccm) of oxygen and 40 sccm of argon gas flow at room temperature. Deposition time of 160 seconds was found to be optimal for the formation of uncoalesced oxide nanoparticles.

The microstructure of the devices was characterized using a scanning electron microscope (SEM) and X-ray diffraction (XRD). Due to the small size of the nanoparticles, the XRD signal from ZnO was not detected. Thus, the analysis was performed on a 300-nm-thick ZnO film sputter deposited on Si (111) substrate with the assumption that the ZnO crystallinity is similar for nanoparticles and for thin films deposited at the identical conditions. Current-voltage characteristics of the devices were also measured to determine the nature of the NW-metal contacts.

For the gas sensing measurements, a device was placed inside the stainless steel chamber with an inlet and an outlet for the analyte vapors. The chamber, with a volume of 0.73 cm³, has a quartz window on the top to facilitate exposure of the device to UV light. The wavelength of the light bulb was confined to the range of 215-400 nm; the intensity recorded at 365 nm was 3.75 nW/cm². The sensor baseline was established at a constant flow of 40 sccm of breathing air under illumination. For sensing experiments, 40 sccm of the mixture of the breathing air and analyte vapor was passed through the chamber. All sensing measurements were performed in the presence of UV light and 5-V dc voltage bias applied across the device terminals. Negligible or no chemiresistive response was observed for all the chemicals in the absence of the illumination.

### Results and Properties

FIG. 6, plate (a) shows a SEM image of a device with a single GaN NW suspended across the metal electrodes. FIG. 6, plate (b) shows the ZnO nanoparticles on the facets of a GaN NW. The current-voltage characteristics of the device measured before and after RTA are shown in FIG. 6, plate (c). As shown in FIG. 6, plate (d), XRD reveals that the sputter-deposited ZnO is crystalline and highly (0002) textured.

Referring to FIG. 8 sensor response to air and nitrogen was evaluated. FIG. 8, plate (a) shows the device response to the different flow rates of breathing air. As seen therein, device conductance decreases upon exposure to the breathing air, and the decrease is proportional to the flow rate. Opposite behavior (i.e., an increase in conductivity) is observed when the device is exposed to nitrogen gas as seen in FIG. 8, plate (b).

Referring to FIG. 7, sensor response to alcohols and other analytes was evaluated. When exposed to alcohol vapors (methanol, ethanol, n-propanol, isopropanol, n-butanol, and isobutanol), the devices showed an increase in conductivity with maximum sensitivity toward methanol. FIG. 7 shows the device response to 500-μmol/mol (ppm) methanol vapor in breathing air.

For the isomers of an alcohol, the sensitivity decreases with branching in the carbon chain. Hence, as shown in FIG. 7 (inset, bottom left), the sensitivity toward isobutanol is less than that toward n-butanol. As shown in FIG. 7 (inset, bottom right), the devices show a negligible response to possible interfering chemicals such as benzene and hexane, whereas the sensitivity toward 100 μmol/mol (ppm) of ethanol is similar to the sensitivity toward 1000 μmol/mol (ppm) of acetone. Ethanol vapor concentration down to 100 nmol/mol (ppb) was successfully detected, and the detection of even lower concentrations is possible with alternative measurement setup.

### Example 4

A hybrid chemiresistive architecture, utilizing nanoengineered wide-bandgap semiconductor backbone functionalized with multicomponent photocatalytic nanoclusters of metal-oxides and metals was demonstrated. These sensors operated at room-temperature via photoenabled sensing.

### Etching of Semiconductor Nanostructures

For real-time nanosensors, successful etching of semiconducting nanostructures, which is characterized by smooth surfaces with minimal sub-surface damage and appropriate side-wall profiles, is desired. This requires overcoming the strong chemical bond energy in widegap semiconductors, and also adjusting the process conditions to overcome inherent defects in epitaxially grown films on non-native substrates using heteroepitaxy. Otherwise, an un-optimized etching process may result in surface morphologies that include pits and/or pillars.

An Inductively Coupled Plasma-Reactive Ion Etching (ICP-RIE) process with Cl₂/Ar/N₂ chemistry is provided, with an etch rate of about 100 nm/min for GaN. The dry etching process may be optimized using X-ray photoelectron spectroscopy (XPS), scanning electron microscopy (SEM), photoconductivity measurements, and photoluminescence (PL) measurements.

### Fabrication Detail

Prior to dry etching, semiconductor wafer surfaces are treated with standard RCA cleaning procedures. As a mask for selective etching, a 500-nm-thick SiO₂ film is deposited by standard plasma-enhanced chemical vapor deposition (PECVD). Etching patterns are defined by deep UV lithography using a proximity aligner capable of generating 300 nm feature sizes. Electron beam deposition of Ni (˜20 nm) followed by lift-off is carried out to complete the formation of mask for the SiO₂ etch.

Direct metal-masking of the semiconductor is not done in order to avoid un-intentional doping of the metal during the etch process. The ICP-RIE etching is performed using the following procedure. GaN etch is accomplished using ICP etching with a Cl₂/N₂/Ar (25:5:2) gas mixture under a pressure of 5 mTorr with varying ICP etching power and radio frequency (RF) power. For nitrides, Chlorine-based etches are used because it has been shown to produce vertical sidewalls due to the ion assisted etching mechanism with smooth profiles. Temperature of the etch is a parameter that provides control of the sidewall angle. With low-temperature etch, the sub-surface damage may also be controlled.

Each sample is treated with a standard RCA clean before the activation annealing, the etching, and the measurements. Etching profile and surface morphology may be investigated by SEM. The surface chemical properties of semiconductor after the etch is characterized using an XPS system and PL measurements performed at room temperature. The electrical properties of etched semiconductor backbone are characterized photocurrent measurements. Photocurrent intensity is a direct measure of the surface recombination, i.e., higher photocurrent intensity will indicate less surface defect non-radiative recombination, hence less sub-surface damage. For GaN, Ti/Al/Ti/Au (70 nm/70 nm/50 nm/50 nm) ohmic electrodes are formed at both ends of the backbone nanostructures and then annealed at temperatures from 500 C to 800 C for ˜1 min. The nanodevices are then functionalized with different metal and metal-oxide nanoclusters using reactive sputtering.

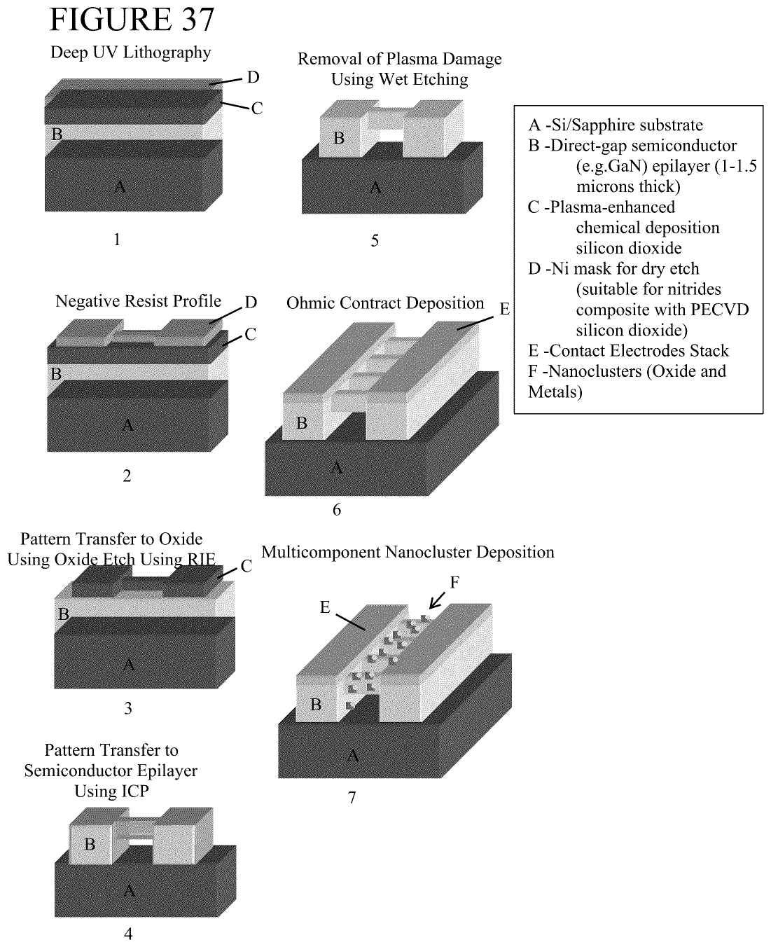

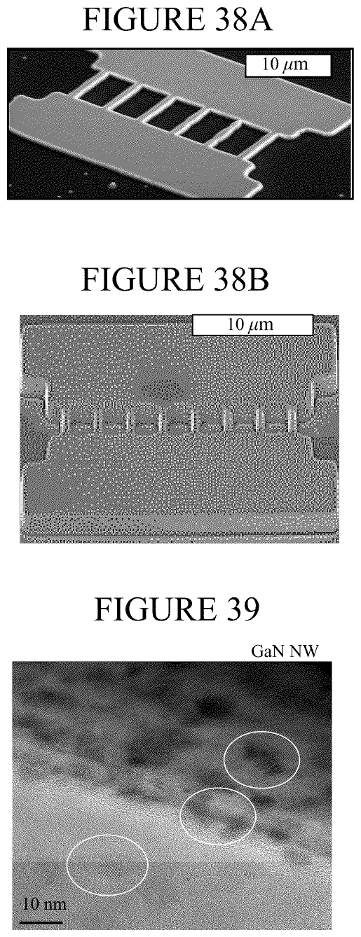

A schematic representation of an exemplary fabrication flow for semiconductor-nanocluster based gas sensors according to the present invention is shown in FIG. 37. As shown, the fabrication flow provides for parallel architecture, with multiple parallel sections. The multi-analyte arrays can be created on one single chip (10 mm×10 mm) by depositing clusters of different components on different micro-scale devices. This is possible due to low-temperature sputtering process used for the cluster deposition. An array of multiple sensors (e.g. for detecting NOₓ, SOₓ, COₓ, NH₃, and H₂O) may be fabricated all on one single chip. FIG. 38 shows exemplary inter-digitated GaN devices on Si and sapphire substrates formed using top-down processes (e.g., such as shown in FIG. 37).

### Example 5

Protection against explosive-based terrorism may be achieved by large-scale production of nano-sensor arrays that are inexpensive, highly sensitive and selective with low response and recovery times. In this study, the selective response of GaN nanowire/TiO₂ nanocluster hybrids to nitroaromatic explosives, including trinitrotoluene (TNT), dinitrotoluene (DNT), nitrotoluene (NT), dinitrobenzene (DNB) and nitrobenzene (NB) at room temperature is demonstrated. The sensors detected between 0.5 ppb and 8 ppm TNT with good selectivity against interfering compounds such as toluene. The sensitivity of 1 ppm of TNT is =10% with response and recovery times of =30 seconds.

N-type (Si doped) GaN nanowires functionalized with TiO₂ nanoclusters were utilized for selectively sensing nitro-aromatic explosive compounds. GaN is a wide-bandgap semiconductor (3.4 eV) with unique properties. Its chemical inertness and capability of operating in extreme environments (high-temperatures, presence of radiation, extreme pH levels) is highly desirable for sensor design. TiO₂ is a photocatalytic semiconductor with bandgap energy of 3.2 eV (anatase phase). The TiO₂ nanoclusters were selected to act as nanocatalysts to increase the sensitivity, lower the detection time, and enable the selectivity of the structures to be tailored to a target analyte (e.g., the most common explosives, trinitrotoluene (TNT) and other nitro-aromatics).

### Materials and Methods

GaN nanowires were grown by Molecular Beam Epitaxy method as described in Bertness K A et al. (2008), supra, J. Crystal Growth 310 (13): 3154-3158. The nanowires are aligned on a pre-patterned substrate using dielectrophoresis. Details of the device fabrication are reported in Aluri G S et al. (2011) “Highly selective GaN-nanowire/TiO₂-nanocluster hybrid sensors for detection of benzene and related environment pollutants,” Nanotechnology 22 (29): 295503. After fabrication of two-terminal GaN NW devices, the TiO₂ NCs were deposited on the GaN NW surface using RF magnetron sputtering. The deposition was done at 325° C. with 50 standard cubic centimeters per minute (sccm) of Ar flow, and 300 W RF power. The nominal deposition rate was about 0.24 Å/s. Thermal annealing of the complete sensor devices (GaN NW with TiO₂ nanoclusters) was done at 700° C. for 30 seconds in a rapid thermal processing system. The device substrates, i.e., the sensor chips, were wire-bonded on a 24 pin ceramic package for the gas sensing measurements.

The microstructure and morphology of the sputtered TiO₂ films used for the fabrication of the sensors were characterized by high-resolution transmission and scanning transmission electron microscopy (HRTEM/STEM), selected-area electron diffraction (SAED), and field-emission scanning electron microscopy (FESEM). For the TEM characterization, the GaN NWs were dispersed on 10 nm thick carbon films supported by Mo-mesh grids, followed by the deposition of TiO₂ NCs and annealing and subsequent Pt deposition. The samples were analyzed in a FEI Titan 80-300 TEM/STEM microscope operating at 300 kV accelerating voltage and equipped with S-TWIN objective lenses, which provided 0.13 nm (STEM) and 0.19 nm (TEM) resolution by points. The instrument also had a Gatan CCD image acquisition camera, bright-field (BF), ADF and high-angle annular dark-field (HAADF) STEM detectors to perform spot, line profile, and areal compositional analyses using an EDAX 300 kV high-performance Si/Li X-ray energy dispersive spectrometer (XEDS).

The as-fabricated sensors were placed in a custom designed gas chamber for gas exposure measurements. Detailed description of the experimental setup and experimental conditions is provided in Aluri G S et al. (2011), supra, Nanotechnology 22 (29): 295503. The device characterization and the time dependent sensing measurements were done using an Agilent B1500A semiconductor parameter analyzer. The gas sensing experiments were performed by measuring the electrical conductance of the devices upon exposure to controlled flow of air/chemical mixture in presence of UV excitation (25 W deuterium bulb operating in the 215 nm to 400 nm range). For all the sensing experiments with chemicals, breathing air (<9 μmol/mol of water) was used as the carrier gas. After the sensor devices were exposed to the aromatic compounds, they were allowed to regain their baseline current with the air-chemical mixture turned-off, without purging or evacuating the test-chamber.

### Results

### Morphological and Structural Characterization of NWNC Hybrids

TEM imaging was conducted under minimal beam intensity conditions close to the Scherzer defocus at highest available accelerating voltage of 300 kV using both stationary beam (bright-field TEM/SAED, phase-contrast high-resolution TEM) and scanning beam (STEM/XEDS) modes. Areas for analyses were selected near the wire's edges and on the amorphous carbon support film in the vicinity of the NWs. FIG. 39 shows HRTEM micrographs of a GaN NW on a thin amorphous carbon support films with TiO₂ coating. The deposited TiO₂ layer formed an island-like film, where 10 nm to 50 nm partially aggregated particles (circled areas in FIG. 39) were often interconnected into extended two-dimensional networks. This was consistent with SAED and compositional analyses of deposited TiO₂ films indicating a mixture of polycrystalline anatase and rutile phases. Despite the limited contrast difference between TiO₂ and GaN, detailed HRTEM and HR-STEM observations revealed 0.35 nm (101) hcp lattice fringes belonging to anatase.

### Current-Voltage (I-V) Characteristics of NWNC Hybrids

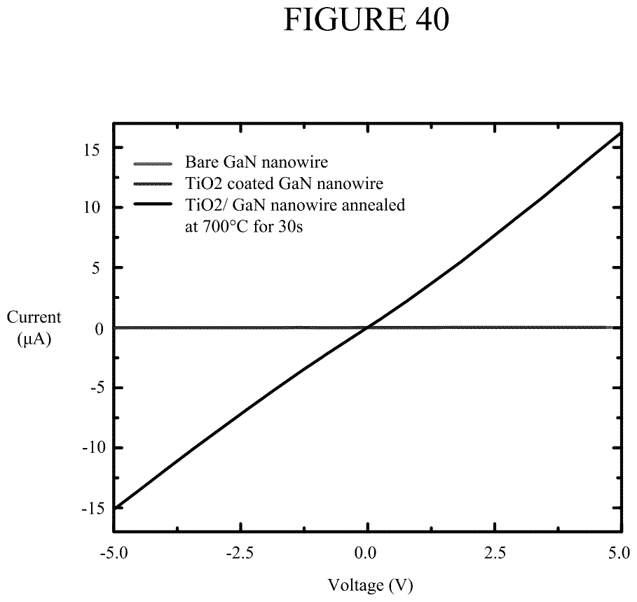

Referring to FIG. 40, I-V characteristics of a GaN NW two-terminal device at different stages of processing are shown. The I-V curves of the as-deposited devices were non-linear and asymmetric (with a low current of 35 nA). However, the current increased (to a 100 nA) with the deposition of TiO₂ nanoclusters. This may be attributed to decreased surface depletion of the GaN NW due to passivation of surface states, and/or the high temperature deposition (325° C.) of the nanoclusters initiating ohmic contact formation. The devices annealed at 700° C. for 30 seconds showed significant changes in their I-V characteristics with a majority of the devices exhibiting linear I-V curves. This is consistent given low resistance ohmic contacts to the nitrides require annealing at 700° C.-800° C.

### Sensing Behavior of GaN/TiO₂ NWNC Hybrid Sensors

The photocurrent through the bare GaN NW devices did not change when exposed to different chemicals mixed in air, even for concentrations as high as 3%. In contrast, the TiO₂-coated hybrid devices responded even to the pulses of 20 sccm airflow in the presence of UV excitation. The response of the TiO₂ NC-coated GaN nanowire hybrid sensors to different concentrations of benzene, toluene, ethylbenzene, chlorobenzene, and xylene in air is discussed above. The GaN/TiO₂ hybrids showed no response when exposed to other chemicals such as alcohols, ketones, amides, alkanes, nitro/halo-alkanes, and esters.

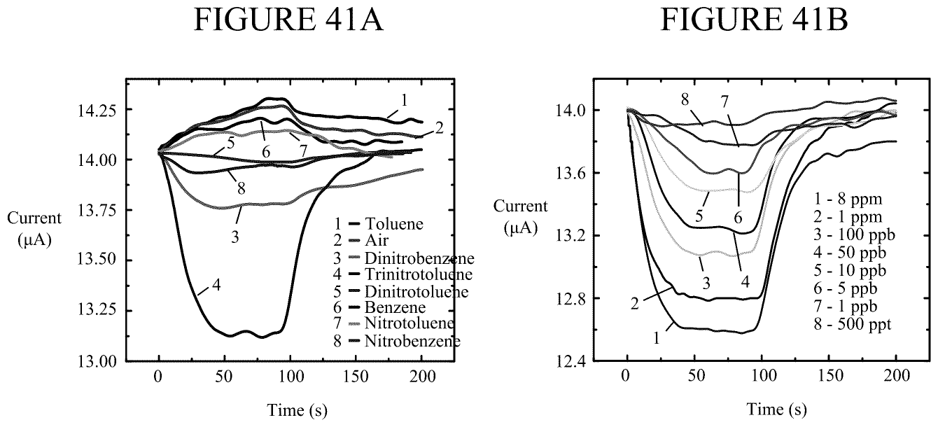

The response of the TiO₂ coated hybrid devices when exposed to a concentration of 100 ppb of the aromatics and nitro-aromatics in air can is shown in FIG. 41, plate (a). The photocurrent for these sensors increased with respect to air when exposed to toluene vapors, whereas for every other aromatic compound the photocurrent decreased relative to air. The response is observed to increase with the increase in the number of nitro groups attached to the aromatic compound. The response of the hybrid device to different concentrations of TNT in air from 8 ppm down to as low as 500 ppt is shown in FIG. 41, plate (b). The response time is defined as the time taken by the sensor current to reach 90% of the response (I_{f}−I₀) when exposed to the analyte. The I_{f} is the steady sensor current level in the presence of the analyte, and I₀ is the current level without the analyte, which in this case is in the presence of air. The recovery time is the time required for the sensor current to recover to 30% of the response (I_{f}−I₀) after the gas flow is turned off. The response and recovery times of the nano-devices to different concentrations of TNT are ≅30 seconds. The response and recovery times of the rest of the compounds varied from ≅60 seconds to ≅75 seconds.

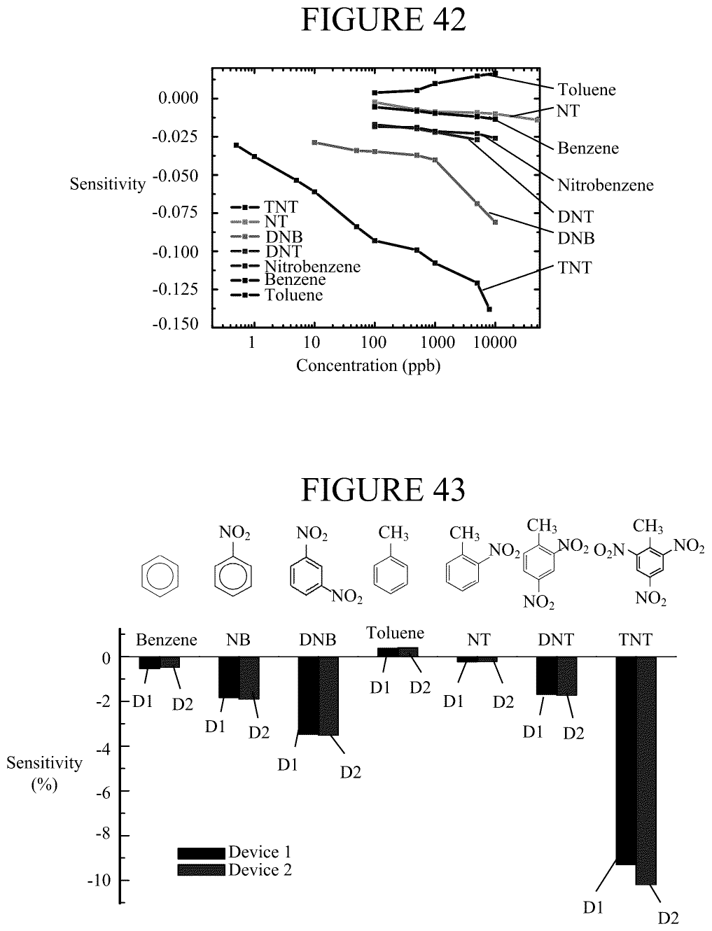

The sensitivity is defined as (R_{gas}−Rₐᵢᵣ)/Rₐᵢᵣ, where R_{gas} and Rₐᵢᵣ are the resistances of the sensor in the presence of the chemical-air mixture and in presence of air, respectively. The sensitivity plot of a hybrid device for the different aromatics and nitro-aromatics tested is shown in FIG. 42. The sensitivity ((R_{gas}−Rₐᵢᵣ)/Rₐᵢᵣ) for 1 ppm of TNT is ≅10%. The devices exhibit a very highly sensitive and selective response to TNT when compared to interfering compounds like toluene. Toluene shows an increase in response with respect to air, whereas TNT shows a decrease when compared to air. The plot identifies the sensor's ability to sense wide concentration ranges of the indicated chemicals. The sensitivity of two different devices (device 1-D1; device 2-D2) to the different aromatic compounds can be seen in FIG. 43.

As discussed above, oxygen vacancy defects (Ti³⁺ sites) on the surface of TiO₂ are the “active sites” for the adsorption of species like oxygen, water, and organic molecules. In the presence of UV excitation with an energy above the bandgap energy of anatase TiO₂ (3.2 eV) and GaN (3.4 eV), electron-hole pairs are generated both in the GaN NW and in the TiO₂ cluster. Photogenerated holes in the nanowire tend to diffuse towards the surface due to surface band bending. This effect of separation of photogenerated charge carriers results in a longer lifetime of photogenerated electrons, which in turn enhances the photoresponse of the nanowire devices in general. Since the nitro-aromatic compounds are highly electronegative, they tend to attract electrons from other molecules through charge transfer. This charge transfer between the adsorbed species on the TiO₂ nanocluster, and the nitro groups in the nitro-aromatic compounds increases the width of the depletion region in the nanowire device, reducing the current.

The potential of the disclosed nanostructure-nanocluster hybrids for next-generation nano-sensors having the capability to detect explosive compounds quickly and reliably is clearly demonstrated. The GaN/TiO₂ nanowire nanocluster hybrid devices tested detected trace amounts of aromatic and nitro-aromatic compounds in air at room temperature with very low response and recovery times (=30 seconds). The nitro-aromatic explosives like TNT are selectively detectable even for concentrations as low as 500 ppt.

### Example 6

Nitrogen dioxide (NO₂) sensors using gallium nitride (GaN) nanowires (NWs) functionalized with titanium dioxide (TiO₂) nanoclusters are demonstrated. Exemplary sensor fabrication methodologies are described above (e.g., see Example 1 & FIG. 19).

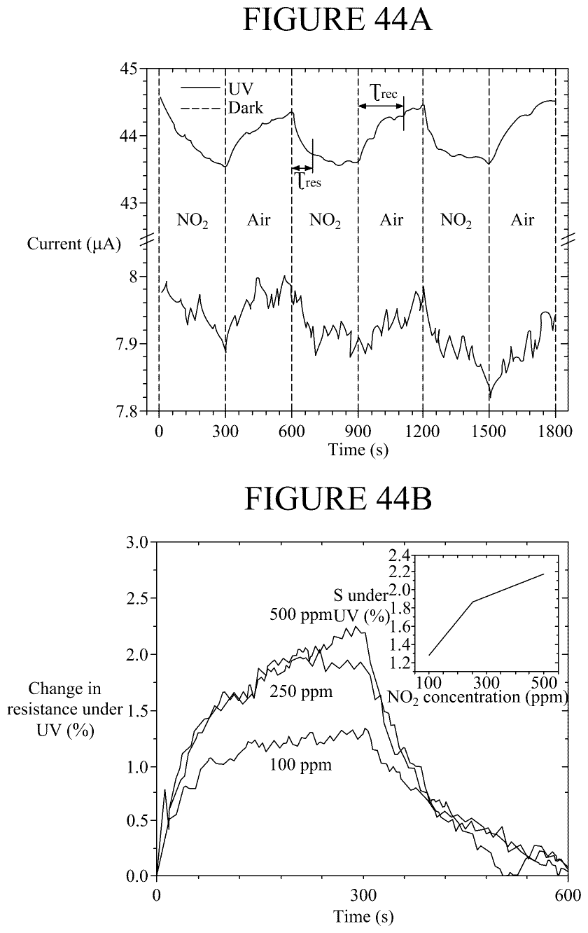

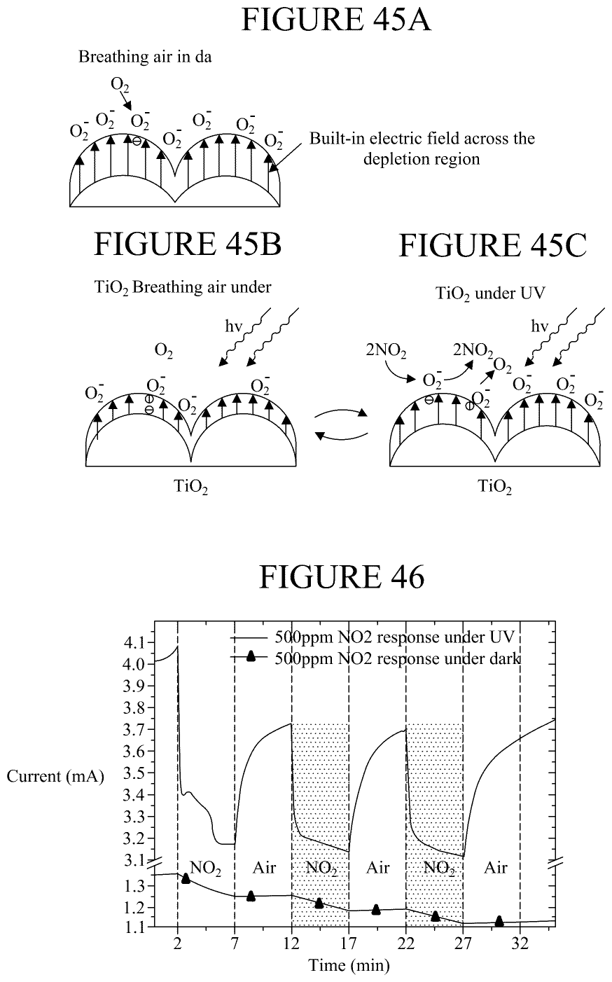

FIG. 44, plate (a) illustrates the dynamic responses of the TiO₂ based sensor exposed to 250 ppm NO₂ mixed with breathing air under UV illumination and dark, and at room temperature. For each cycle, the gas exposure time was 300 s. FIG. 44, plate (b) illustrates change in resistance under UV at mixtures of 100 ppm, 250 ppm, and 500 ppm with breathing air, with the inset showing the measured responses under UV as a function of NO₂ concentrations with uncertainty. Sensitivity S is presented by (I_{g}−Iₐ)×100/I_{α}, wherein I_{g} is the device current in the presence of an analyte in breathing air and I_{α} is the current in pure breathing air, both measured 300 s after the flow is turned on. FIG. 45 illustrates schematically an NO₂ gas sensing mechanism of the TiO₂ sensor under UV illumination and at room temperature. FIG. 45, plate (a) shows the mechanism in a dark environment with breathing air in. FIG. 45, plate (b) shows the mechanism under UV illumination in breathing air. FIG. 45, plate (c) shows the mechanism under UV illumination with a mixture of NO₂ and breathing air.

The response of the TiO₂ based sensor exposed to 500 ppm NO₂ under UV illumination and under dark at room temperature is shown in FIG. 46. As described, the UV illumination allows for efficient photodesorption of adsorbed oxygen and hydroxyl species, thus introducing additional oxide surface sites or receptors for adsorption of target molecules. Photocatalytic reactions then occur between the adsorbed target molecules and the photo carriers in the oxide sites, leading to a modification of the surface potential and semiconductor backbone current change (transduction). The photodesorption of the adsorbed target molecules and reaction species leads to a reversal of photocurrent to baseline (recovery).

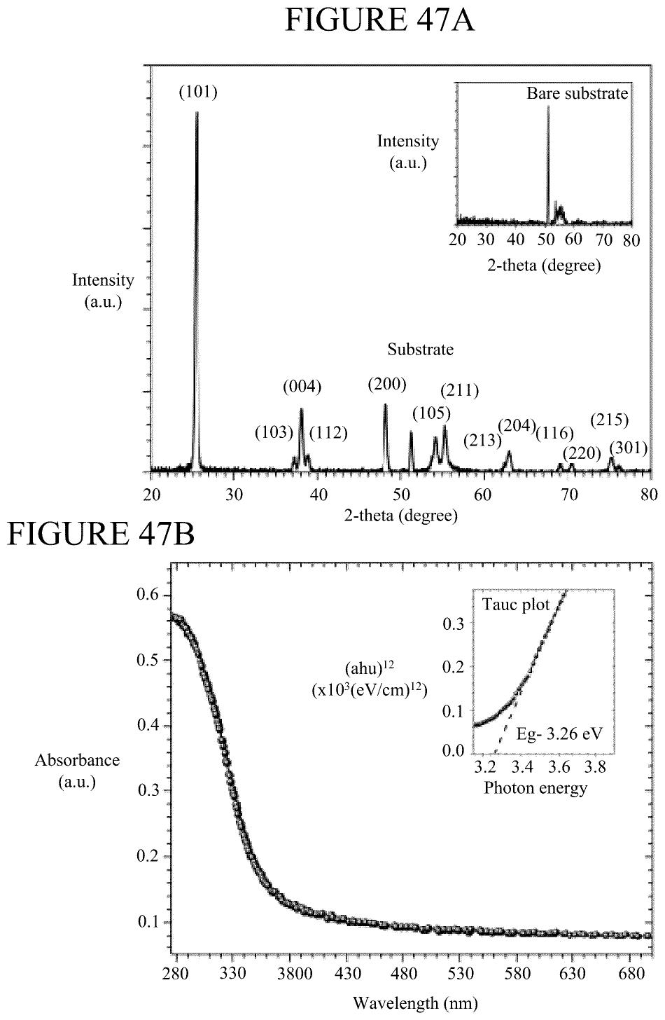

A GIXRD scan of thermally processed ultrathin TiO₂ film is shown in FIG. 47, plate (a). Optical properties (bandgap) are illustrated in FIG. 47, plate (b).

### Example 7

Carbon dioxide (CO₂) sensors using gallium nitride (GaN) nanowires (NWs) functionalized with tin oxide and copper oxide (SnO₂—CuO) nanoclusters are demonstrated. Exemplary sensor fabrication methodologies are described above.

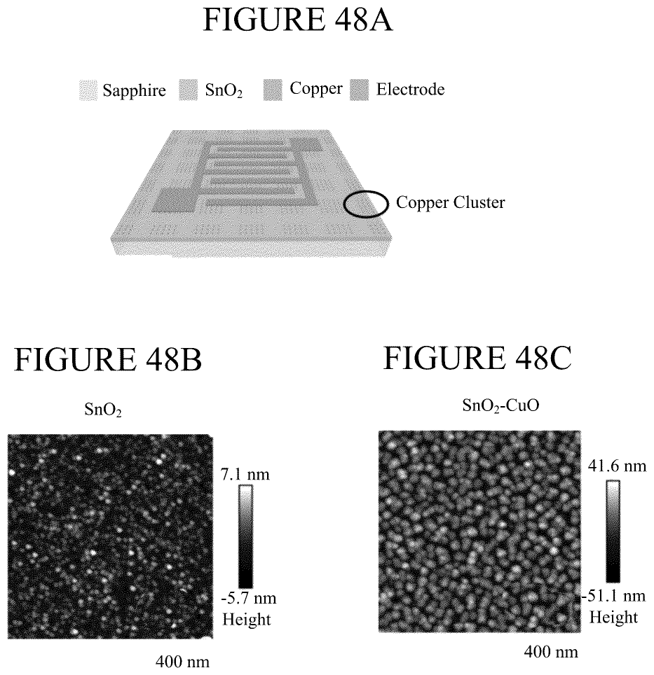

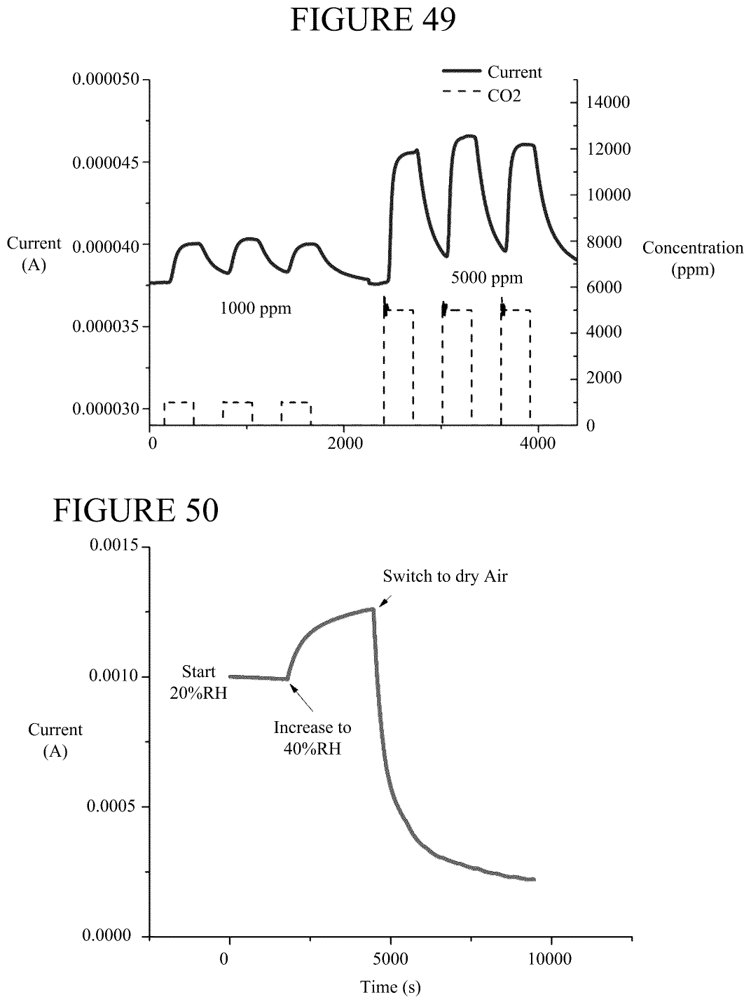

FIG. 48, plate (a) illustrates schematically a SnO₂—Cu nanocluster CO₂ sensor, including an electrode disposed on a sapphire substrate, and GaN NWs functionalized with SnO₂ nanoclusters and SnO₂—CuO nanoclusters. AFM images of the SnO₂—Cu nanocluster CO₂ sensor are shown in FIG. 48, plates (b) and (c). FIG. 49 illustrates the dynamic response of the SnO₂—Cu based sensor exposed to CO₂ at room temperature for various concentrations. FIG. 50 illustrates graphically the response of the SnO₂ based sensor at different relative humidity (RH) concentrations at room temperature.

### Single Package Optically-Activated Gas Sensors

As described in the Background section, it would be desirable to provide systems, methods and devices which address the deficiencies of, e.g., currently manufactured and packaged optically-activated gas sensors. Prior to describing embodiments associated with single package gas sensors, examples of chips, sensing elements and sensor structures which can be used inside of the single package gas sensors will first be described.

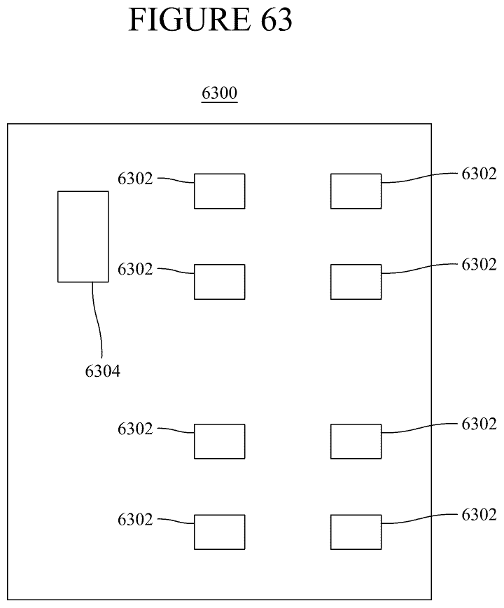

According to an embodiment, chip-scale gas sensors can be two-terminal photoconductors, surface functionalized with nano-scale photocatalytic material. An example of a sensor 6300 used for gas sensing is shown in FIG. 63. This sensor 6300 includes eight individually addressable microsensors 6302 as well as various calibration and compensation elements 6304. The sensor 6300 can have, for example, a trapezoidal cross-section which can have a top-width of ˜250 nm and is formed by top-down etching. Various combinations and quantities of the individually addressable microsensors 6302 can be used as desired depending upon the application the sensor 6300 is to be used in. For more information regarding the chips (or dies) including materials and the manufacturing of the chips, the interested reader is directed to the various documents incorporated herein by reference.

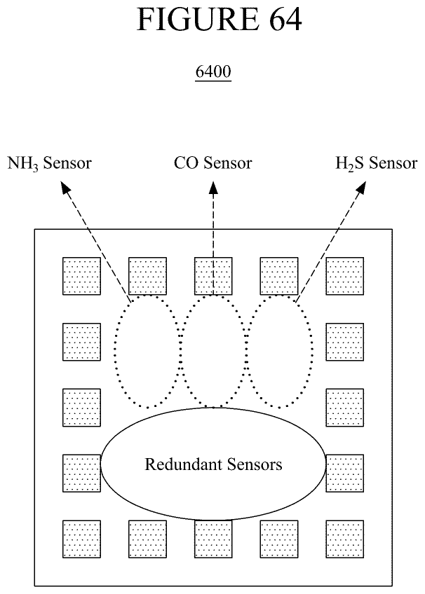

According to another embodiment, a multi-gas sensor for detecting the presence of multiple gases or compounds can also be used in a single-package sensor. An example of a multi-gas sensor which can be used in a single-package sensor is shown in FIG. 64. FIG. 64 shows a die 6400 which has a plurality of sensors for detecting the presence of different compounds, more specifically there is a NH₃ sensor 6402, a CO sensor 6404 and a H₂S sensor 6406. Additionally, there can be more than one sensor of each type as represented by the redundant sensors 6408.

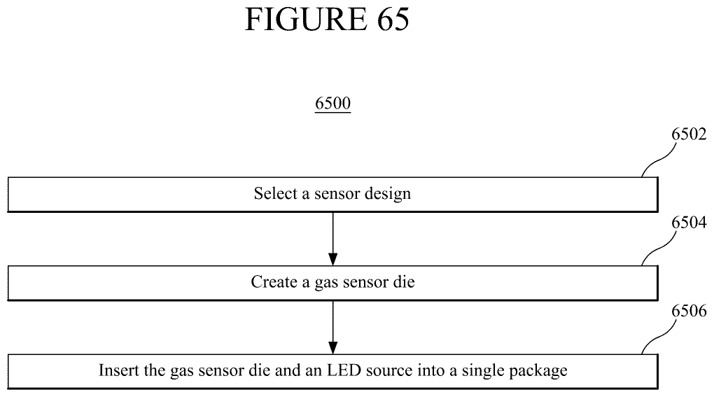

As described above, there are many different options for what type of materials to create the gas sensors from depending upon the desired end use of the gas sensors. An overview of an example of a process resulting in a single package gas sensor is now described with respect to the flow diagram 6500 shown in FIG. 65. Initially, in step 6502, a sensor design is selected based upon the desired end use. Using, for example, the processes described in the '862 patent or the other related, referenced pending patent applications, a wafer is processed to include etching and functional material deposition to create a gas sensor die in step 6504. In step 6506, the gas sensor die and an LED source are inserted into a single package.

Of particular interest in this application, are embodiments associated with various facets of the single package to include, but not be limited to, integration, disposition of components, filtering, assembly, materials and packaging. Embodiments addressing these areas are described below in more detail.

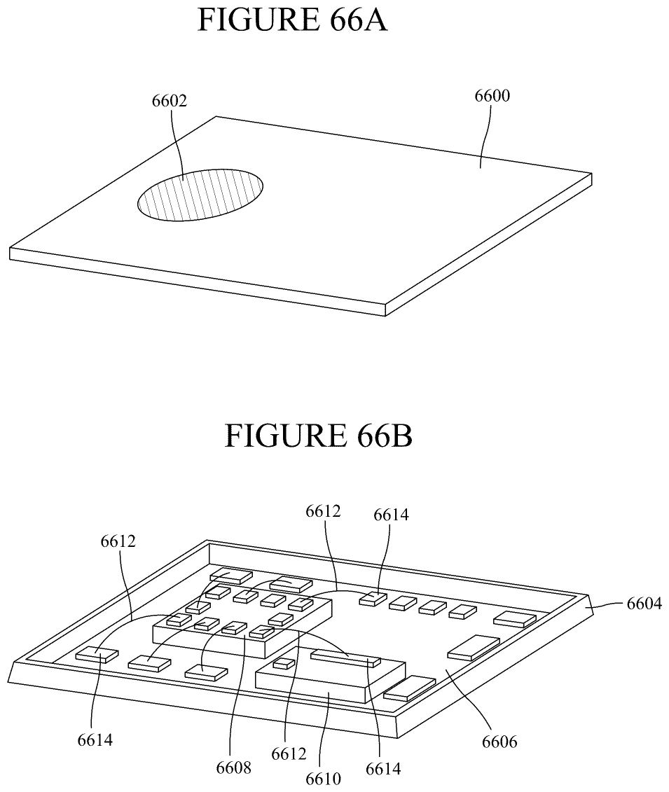

According to an embodiment, an example of a single gas sensor package prior to assembly completion is illustrated in FIGS. 66(a) and 66(b). FIG. 66(a) shows a lid 6600 with an optional filter 6602. FIG. 66(b) shows an exterior housing 6604 in which there is a sensor die 6606 which acts as a base upon which other components are located. Disposed on the sensor die 6606 there is a gas sensor die 6608, and an LED die 6610. which can be a UV light source of varying wavelength. Additionally, in various places on the various dies, there are a plurality of electrical connections 6612 for connecting the components which need power to a plurality of electrical pads 6614.

When the LED die 6610 is a UV LED die, various options exist with respect to power output, power usage, size and emitted wavelength for use depending upon the application. For example, for one application the LED die emits light with a peak wavelength of 365 nm. However, for other application, different wavelengths of emitted light can be preferred. Additionally, multiple LED dies which emit different wavelengths of light can be disposed on a single sensor die 6606 in support of, for example, activating gas sensor dies which are designed to detect different types of gases. This can provide data which can improve such features of the single package gas sensor as accuracy, detection limit, detection speed, etc. According to another embodiment, the intensity of the emitted light can be varied in a real time fashion based on, for example, input from the user of the gas sensor.

According to an embodiment, regarding the wavelength of the emitted light from the LED die 6610, it is too be understood that varying wavelengths can be used depending upon the composition of the material of the photoactivatable portion of the gas sensor die 6608, e.g., the emitted light can be in the range of ultraviolet light (100 nanometers (nm)) to roughly the end of the infrared spectrum (approximately 1 millimeter (mm)). Additionally, according to an embodiment, a desired diffusion of the emitted light from the LED die 6610 is where the maximum photon flux is reflected on the sensor die for the lower amount of optical power emission from the LED.

According to another embodiment, one can package more LED dies 6610 and also more gas sensor dies 6608 as well as providing the LED dies 6610 with different wavelength emission capabilities on the same package giving the ability to package different types of gas sensors on single package. Alternatively, a single package can include LED dies 6610 of different sizes and power requirements. The LED die 6610 can include a built-in Zener diode for both electrostatic discharge protection and reverse bias protection.

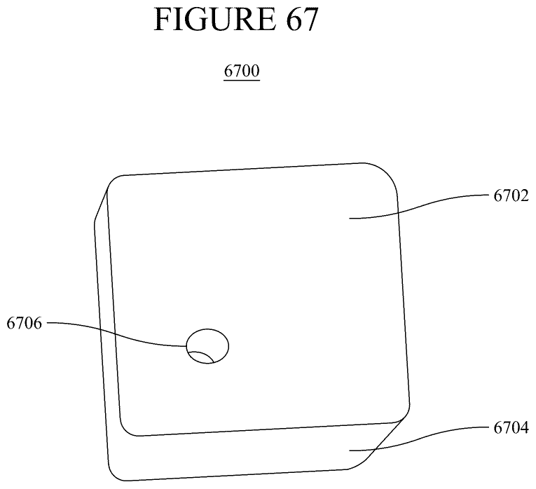

According to an embodiment, FIG. 67 shows an image of a single package gas sensor 6700, which includes the gas sensor die and the UV LED die (not shown in this image as they are located within the housing). The single package gas sensor 6700 as shown has a lid 6702, a housing 6704 and at least one gas ingress location 6706 on the lid 6702. According to an embodiment, the gas ingress location 6706 can be fitted with a filter to reduce dust, for use in humid conditions, condensing humidity as well as providing general protection for the internal dies. According to an embodiment, the filter can also be an active filter in that the filter provides filtering of different molecules to provide more selectivity of the sensing device. For example, the gas sensor is designed to detect CO₂ but the gas sensor has some cross sensitivity to CO, the filter can be designed to filter CO while not impeding or perhaps minimally impeding the CO₂. Additionally, according to an embodiment, the lid 6702 is made of a material which can reflect, at least internally to the gas sensor, light emitted from LED die 6610. The lid 6702 can be flat or have a dome shape. Examples of such a material for the lid 6702 include aluminum, steel and tin. Examples of materials from which the housing 6704 can be made include plastic, Teflon, ceramics, various metals and combinations of these items, e.g., a Teflon coating on a metal.

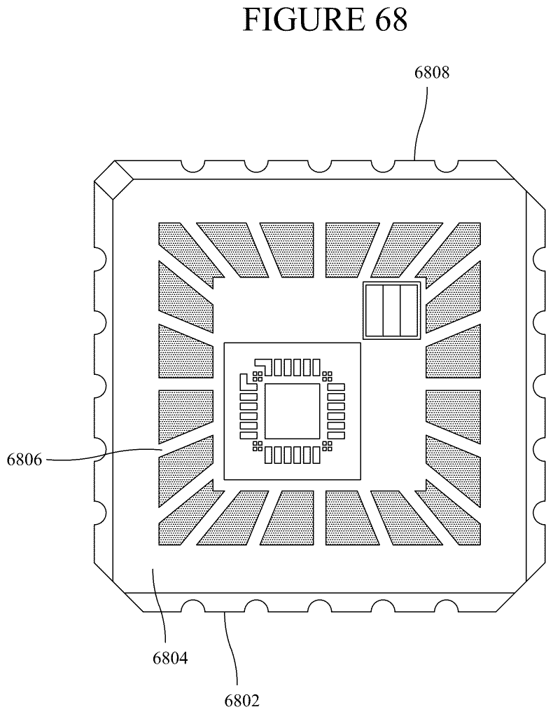

FIGS. 68-70 show additional sketches of examples of portions of the single package gas sensor. More specifically, FIG. 68 shows the housing 6802, the sensor die 6804, the gas sensor die 6806 and the UV LED die 6808. In this example, the location of the UV LED die 6808 relative to the gas sensor die 6806 is selected to represent the location of the UV LED die 6808 such that when powered to emit UV light, the UV light is reflected off of the lid such that the gas sensor die 6802, and their associated individually addressable microsensors 6302 (not shown in FIG. 68), are activated by the emitted, reflected UV light.

According to an embodiment, gas sensor die 6806 (or in some cases multiple gas sensor dies) are present and activated by receiving UV light. This light is typically reflected off of the lid prior to the light being received by the gas sensor die 6806. Activating, in this context, describes the active sites of the gas sensor die 6806 are enabled to detect a target analyte only when receiving the UV light. Regarding a placement relationship of the LED die 6808 and the gas sensor die 6806, the LED die 6808 can be placed next to the gas sensor die 6806, at a corner of the gas sensor die 6806, mounted directly on top of the gas sensor die 6806 or at another location which provides for the desired emission, reflection and diffusion of the light for activation of the gas sensor die 6806.

The LED die 6808 can be further mounted using “flip-chip” bonding, where in the LED die is flipped on its bottom surface and mounted directly on the sensor die, allowing to LED to excite the catalytic surface directly, without the use of reflecting lid, further simplifying the packaging, making the package smaller and lower cost as the entire package can be made from plastic. Further, the relative position of the place of the LED die 6808 to the gas sensor die(s) 6806 can depend on many factors, such as, shape and size of the package, lid material, gas sensor die 6806 size and shape thickness, LED power and LED die 6808 size, and thickness, and can be tailored depending on the application also.

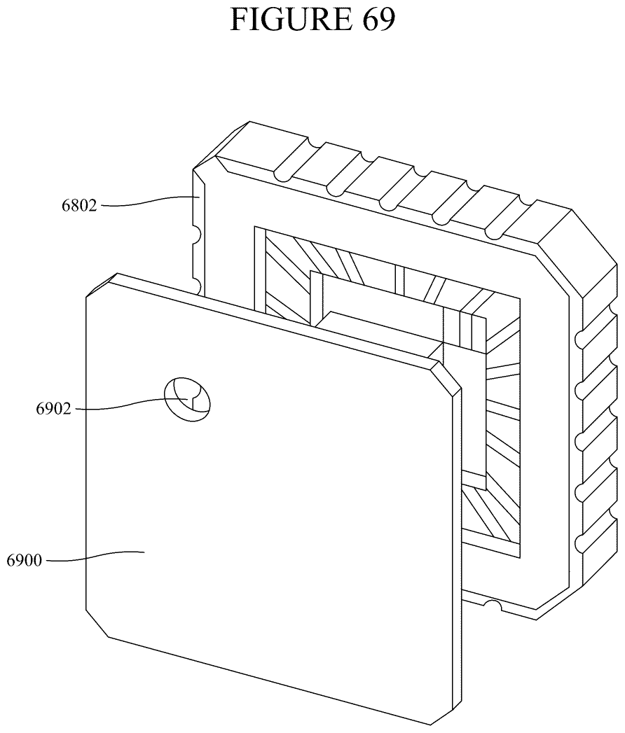

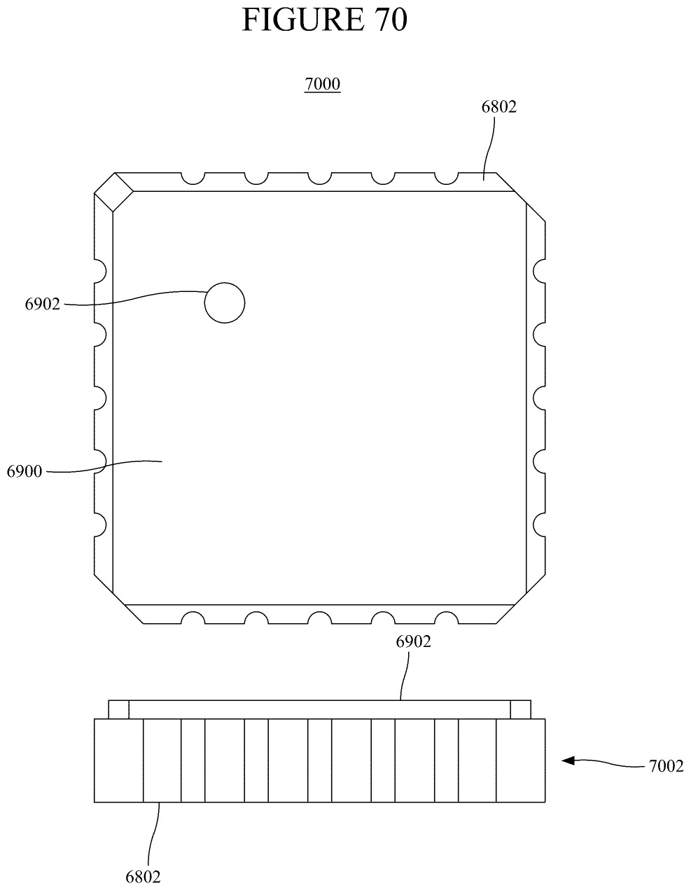

Additionally, while not shown in FIG. 68 there can be a plurality of gas sensor dies 6806 all of which would be activated by the reflected UV light. FIG. 69 shows a lid 6900 with a gas ingress point 6902 and the housing 6802 prior to the lid 6900 and the housing 6802 being assembled together. FIG. 70 shows the single gas sensor package 7000 and a view of the single gas sensor package's cross section 7002. The lid 6900 and the housing 6802 can be attached, for example, through the use of an adhesive. However, other attachment methods can also be used as desired.

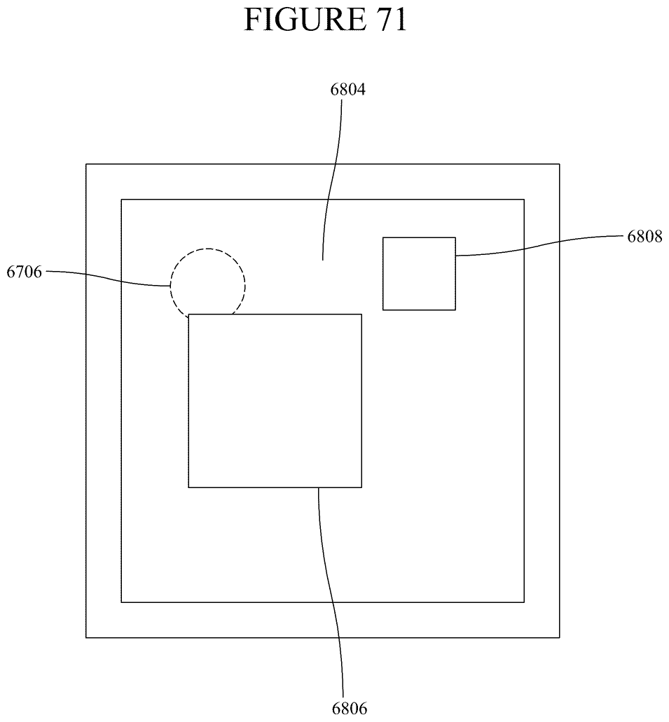

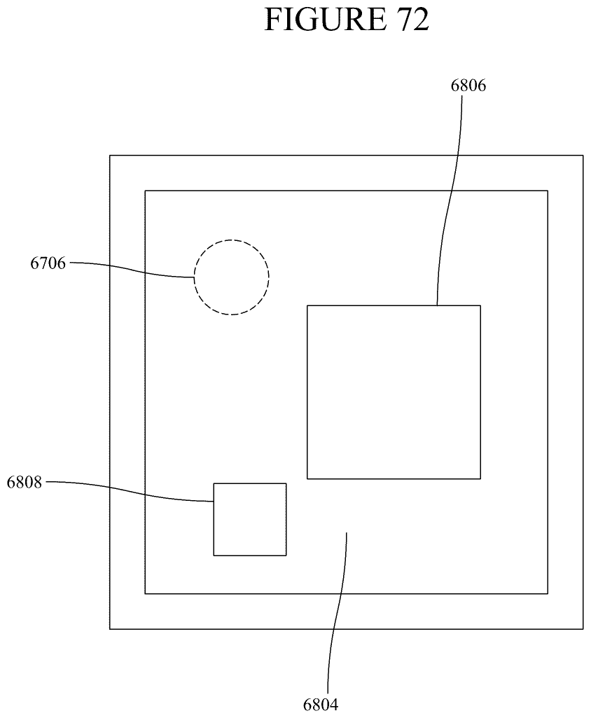

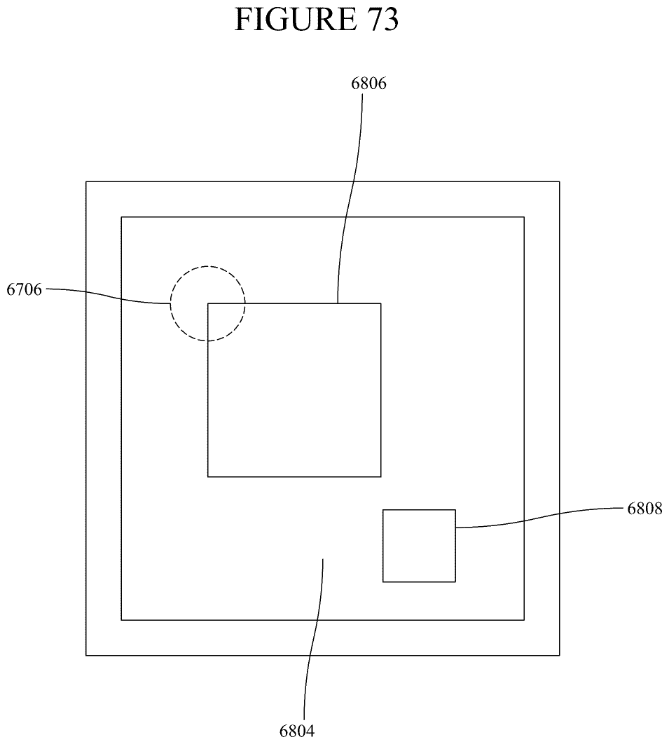

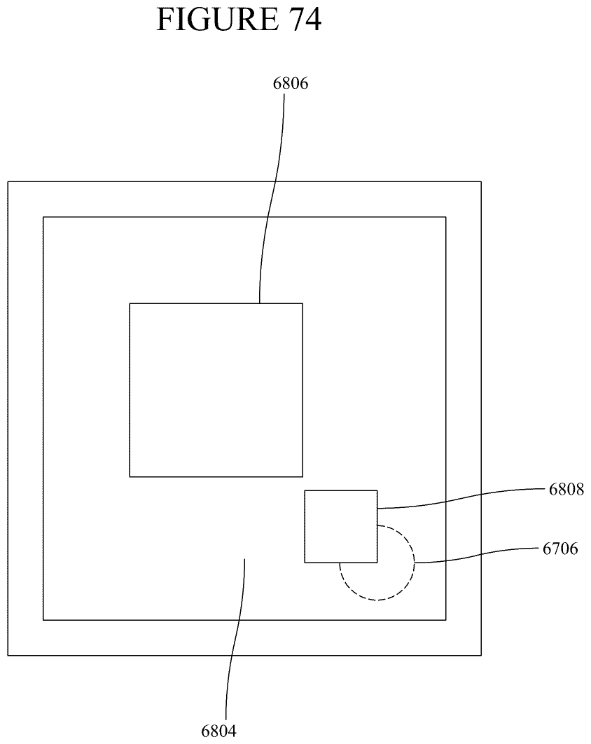

According to an embodiment, the relative position of the LED die(s) 6808 and the gas sensor die(s) 6806 are selected to allow for a desired diffusion of the reflected light. This occurs when the desired diffusion occurs where a maximum, or near maximum, photon flux is reflected onto the gas sensor die 6806 using a lowest, or relatively low, effective amount of optical power emission from the at least one UV LED. Physically this can be implemented as shown in FIGS. 71 and 72. FIGS. 71 and 72 show placement the LED die 6808 and the gas sensor die 6806 on the sensor die 6804 as well as a representation of the gas ingress location 6706. In FIGS. 71 and 72 it is noted that when looking down on the sensor die 6804, for increasing or maximizing photon flux reflected onto the gas sensor die 6806, the placement of the gas ingress location 6706 is in such a place as to not be overtop of either the gas sensor die 6806 or the LED die 6808 as this can reduce reflectivity. FIGS. 73 and 74 show undesirable setups as the gas ingress location 6706 placement overlaps either the gas sensor die 6806 or the LED die 6808 when looking down upon the gas sensor 6804. Further, according to an embodiment, these concepts can also be implemented, or implemented as best able, when a plurality of one or more of the gas sensor dies 6806, the LED dies 6808 and the gas ingress locations are present.

As described above, embodiments describe a gas sensor die and a UV LED both disposed on a same, single chip. The gas sensor die used can be a single gas sensor or a multiple gas sensor on a single chip. Various different gas sensors can be used within the described packaging. For example, one product can be a NO₂ and CO₂ sensor on one chip as an indoor air quality monitoring device. For another example, another product could be a Cl₂ and HCl sensor for toxic industrial chemical detection. Other sensor examples include, but are not limited to, H2/Methane for explosive gas sensing, H₂S/CO for wastewater treatment, NH₃ for cold storage monitoring and H2 for fuel cell based applications. Additionally, other sensing options, as described above and in the incorporated by reference documents, can also be used in the single sensor package. Advantages include, but are not limited to, providing gas sensing on a chip-scale package, reduced costs compared to traditional sensors, sensors which can selectively detect compounds without being effected by the presence of other interfering gases, sensors which are robust with respect to temperature and humidity changes, sensors which have reduced degradation when exposed to corrosive gases and the ability to provide multi-gas detection on a single chip device.

According to an embodiment, while this description of the packaging has focused on single package gas sensors, it is too be understood that the single packaging concept can be implemented with other types of sensor, particularly where a sensor and a light (or other type of emitter) source are to be in a single package. For example, some alternative applications can include UV/photodetection, radiation detection, temperature sensing, humidity sensing. Similar architecture and modifications can also be used for detection of specific molecules in a liquid medium, such as, mercury, lead, other heavy metals in water or other liquid mediums. According to another embodiment, a modified packaging architecture with sensor(s) can also be used for detection of biomarkers in blood, detection of biological agents, e.g., viruses and bacteria, in a liquid medium as well as detection of chemical species in a liquid medium such as water, blood and other biological medium.

According to an embodiment, a control logic can be implemented by a processor, microprocessor and the like, for managing the power consumption of the LED die 6806, as well as the entire single package gas sensor 7000, as well as response time and recovery times. LED intensity for some detectable gases is related to response and recovery times. This means that higher intensity LED light can make the sensor sense faster and to also refresh faster which, in some scenarios, could lead to being able to sense smaller amounts of the desired chemical in atmosphere that is lower than its traditional detection limit. However, as increasing the LED intensity increases total power consumption, so embodiments also allow for real time LED intensity variation by an operator to optimize the single package gas sensor 7000 to the user's needs.

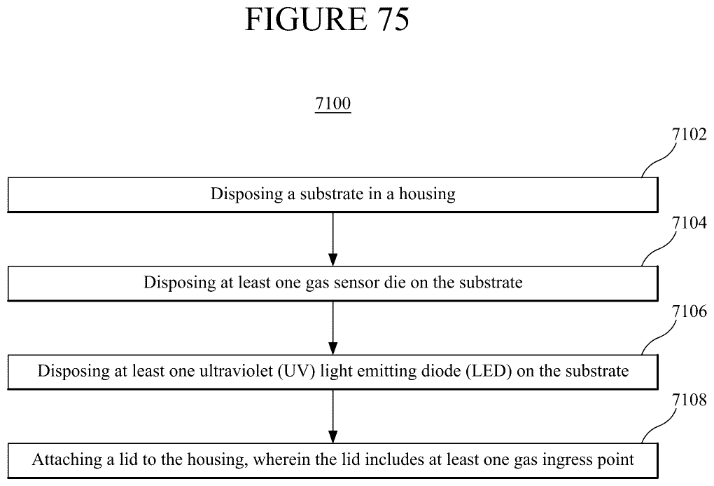

According to an embodiment, there is a method for assembling a single package gas sensor as shown in the flowchart 7100 of FIG. 75. In step 7102, disposing a substrate in a housing; in step 7104, disposing at least one gas sensor die on the substrate; in step 7106, disposing at least one ultraviolet (UV) light emitting diode (LED) on the substrate; and in step 7108, attaching a lid to the housing, wherein the lid includes at least one gas ingress point.

### Application Specific Integrated Circuit (ASIC) Controllers for Gas Sensors

Embodiments describe using an ASIC as a controller for controlling operation of the afore-described sensors (or other sensors), optionally in a single package (as described above). One of the reasons that a controller is needed for gas sensors such as those described above is that the fabricated bridge sensors will have an unknown baseline resistance level which can vary by thousands of kΩ. This causes an initial offset in the differential bridge voltage which is then amplified by the analog front end of the chip. A change in sensor output will thus be limited if the offset is large and causes the signal to saturate, providing less accurate gas concentration readings. Thus according to these embodiments, the ASIC is designed to automatically calibrate out any offset such that the sensor output starts at mid-rail or zero.

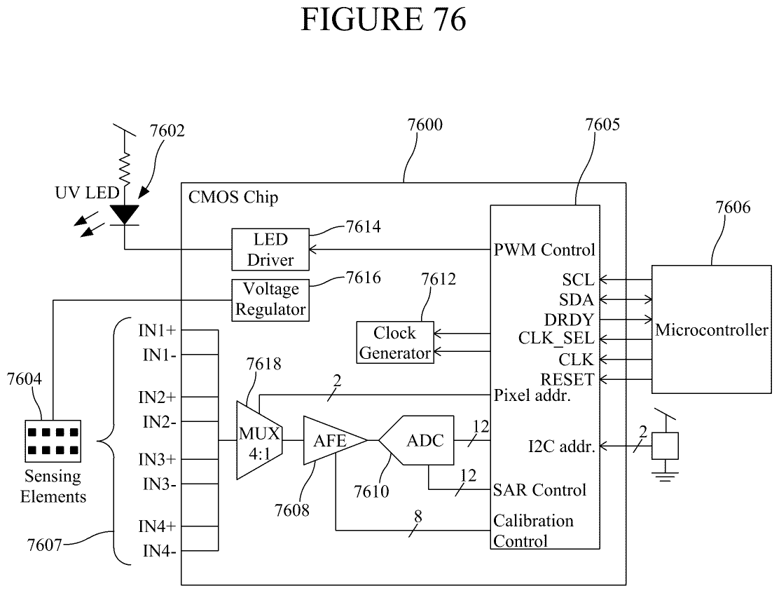

A block diagram of an ASIC controller according to an embodiment is shown in FIG. 76. Therein, the ASIC 7600 is electrically connected to a UV LED 7602 and gas sensing elements 7604, e.g., wherein the UV LED 7602 provides UV light onto the gas sensing elements 7604 to actuate the gas sensing elements 7604 as described above. Internal to the chip 7600, control of the components is handled by an internal controller 7605 which is disposed on the chip 7600. Optionally, as described in more detail below, the ASIC 7600 may also be connected to an external microcontroller 7606 to control on-chip operation. According to this embodiment, an ASIC 7600 includes a number of components, for example, an analog front-end (AFE) which includes offset cancellation elements and amplification of bridge sensor outputs, a successive approximation analog-to-digital converter (ADC) 7610, a clock generator 7612, an LED driver 7614, and sensor supply voltage regulator 7616. The functionality of these components will be described in more detail below. As will be appreciated by those skilled in the art, ASIC 7600 will also include other elements which are not shown in FIG. 76, e.g., other miscellaneous circuits including on-chip bias generators and buffers, and digital logic.

In operation, voltage regulator 7616 sources power to the gas sensing element 7604 (also sometimes referred to herein as “chemiresistors”. Periodically, the LED driver 7614 drives the UV LED 7602 to stimulate the chemiresistors 7604, under control of the PWM control signal generated by internal controller 7605 to control the duty cycle of the LED driver signal, and then the resistance of those sensing elements 7604 upon exposure to the UV light is measured. To this end, the LED driver 7614 can include a constant-current driver (not shown) which is designed to sink 10 mA of current and an amplifier (not shown) which is used to modulate the gate voltage of a PMOS such that its drain voltage matches a predefined reference level. The resulting gate voltage is buffered to a large PMOS which acts as the LED current sink in series with a large NMOS transistor that switches on and off with the PWM signal. A PWM driver is used to increase the transition-time of the PWM signal to increase stability. In this embodiment, there are four bridge pairs 7607 of chemiresistors with plus and minus channel outputs from each bridge pair (i.e., IN1+/IN1−, IN2+/IN2−, IN3+/IN3− and IN4+/IN4− in FIG. 76) via which the resistance of each gas sensor in the set of four is measured.

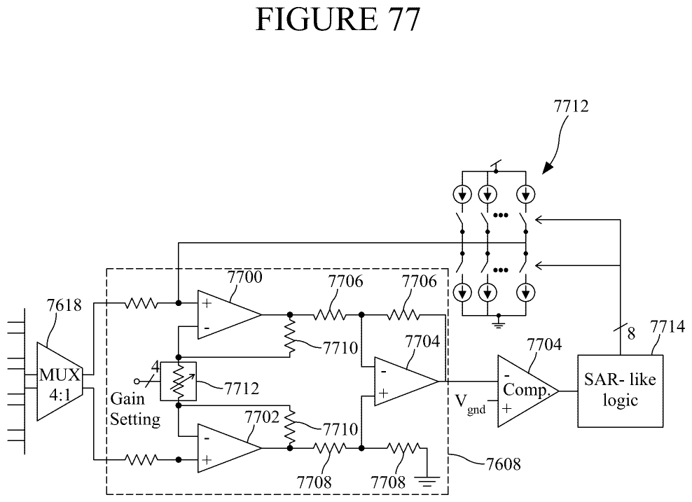

Under the control of the internal controller 7605 via the Pixel addr. signal line, one of the four channels is selected by multiplexer 7618 for input to the amplifier 7608 in the analog front end of the chip 7600. The gain level for the amplifier 7608 is set by the calibration control signal from internal controller 7605 to provide calibration of the gas sensors' outputs as will be described in more detail below. A more detailed view of this portion of the ASIC 7600's circuitry according to an embodiment is provided in FIG. 77. Therein, a 3 op amp instrumentation amplifier topology was chosen for amplifier 7608 due to its small size, simplicity, and ability to achieve relatively low gains. As seen in dotted block 7608, it consists of three op amps 7700, 7702, 7704, three pairs of resistors 7706, 7708, 7710, and a bank of four resistors 7712 that can be programmed to set the overall amplifier gain. The four pairs of gas sensor inputs are multiplexed using an analog mux 7618 and fed into the instrumentation amplifier 7608.

As mentioned previously, the amplifier 7608's output needs to be calibrated at the start of a measurement cycle. This is because the fabricated gas sensors 7604 will have varying initial resistances which lead to an unknown amplifier output. So in order to calibrate each sensor, the ASIC 7600 evaluates the output of the amplifier 7608 when the sensor is not exposed to any gas and marks that output as the zero-start level. Depending on the final use case, the ASIC 7600 can then adjust the zero-start level to be near 0 V, or at the middle of the supply rails (Vad/2 V). Choosing this target value is done by setting Vgnd (at the positive input of the comparator 7710 seen in FIG. 77). The actual adjustment is made by switching on and off a bank of current sources 7712 at the Vref node of the instrumentation amplifier 7608. These switches are controlled using digital logic 7714 that uses a SAR-like binary search algorithm to iteratively switch on the current sources 7712 from largest to smallest. The decision to keep or discard each current source is set by the comparator's output. As such the comparator 7710 is designed to discriminate small differences in its inputs and able to operate near the supply rails.

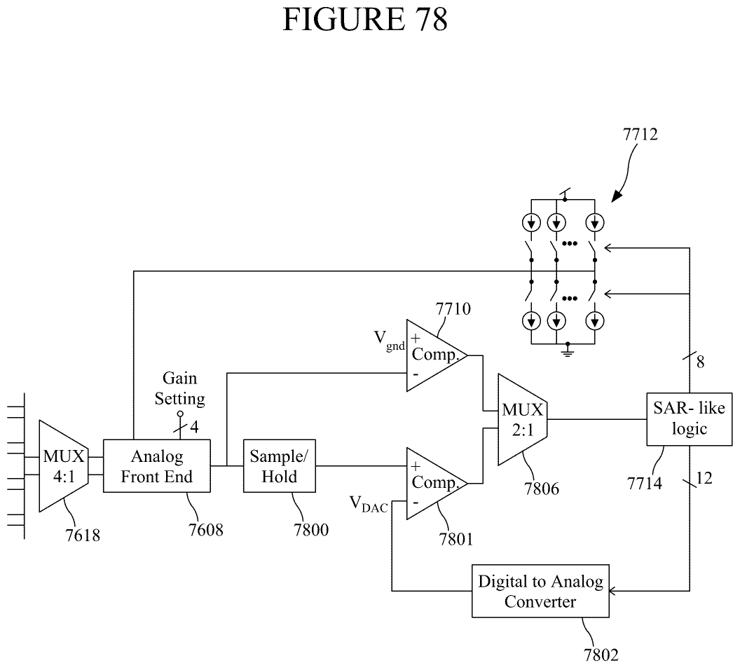

The output of the analog front end 7608 is digitized, according to this embodiment, using a 12 bit successive approximation analog-to-digital converter (ADC) 7610. As seen in FIG. 78, which extends on elements illustrated in FIG. 77, the signal (stored in sample and hold 7800) being digitized is compared at comparator 7801 to the output of a digital to analog converter (DAC) 7802 whose value is controlled by the SAR logic 7714. The output of the DAC 7802 is changed in a binary search fashion by logic 7714 until it converges to the value of the input signal. Multiplexer 7806 switches between the aforementioned evaluation of the current source decisions and the comparison between the ADC/DAC signals toward the logic block 7714.

Regarding the internal controller 7605, this controller can operate in either an internal control mode or an external control mode (if the optional external controller is used). When operating in internal control mode, after calibration, the internal controller 7605 waits a specified sampling interval, activates the PWM control signal to activate the LED driver 7614 and UV LED 7602, waits for a ramp-up duration, then takes a measurement of each of the four inputs, flags microcontroller to read, then goes to a wait mode until the next sampling interval. When operating in external control mode, after calibration, the internal controller 7605 goes into a wait mode, an external trigger can be sent by the external microcontroller 7606 to initiate a read of all four inputs and the measurements are saved into registers. The external controller 7604 can then read the registers through the I2C bus.

As can be seen in FIG. 76, other than the UV LED 7602 which is used to activate the sensing elements 7604, the sensing elements 7604 themselves and the optional external microcontroller 7606, all of the components needed to control the UV LED 7602 and to calibrate the sensing elements 7604 are disposed on a single chip 7600. Regarding the optional microcontroller 7606, if provided, this element enables the ASIC 7600 to receive instructions externally for calibrating the amplifier 7608 and operating the UV LED 7602. Providing an external microcontroller 7606 enables, for example, the gas sensors to 7604 to be queried for gas related data at a time and manner which is controlled by an external entity. The arrows shown in FIG. 76 between the external microcontroller 7606 and the internal controller 7605 represents signals conveyed between the two controllers via, for example, an I2C bus. For example, the SCL and SDA signals represent the clock and data signals transmitted by the external microcontroller 7606 to the internal controller 7605 via the I2C bus. The DRDY signal flags the internal controller 7605 to read the data. The CLK_SEL signal allows the microcontroller 7606 to instruct the ASIC 7600 to either use its internally generated clock signal (via clock generator 7612) or to use the clock signal sent by microcontroller 7606 for clocking the various electrical components on the chip. Lastly, the RESET signal enables microcontroller 7606 to reset the values used by chip 7600 to baseline values.

From the foregoing, it will be appreciated that some embodiments include the following features: (a) each of the four sensor inputs has individual calibration settings since it is unlikely that multiple sensors will have identical initial characteristics and, therefore, during a readout, the calibration settings are loaded separately for each sensor; (b) each of the four sensor inputs has individual gain settings; and (c) each of the four sensor inputs has its own individual zero-level setting.

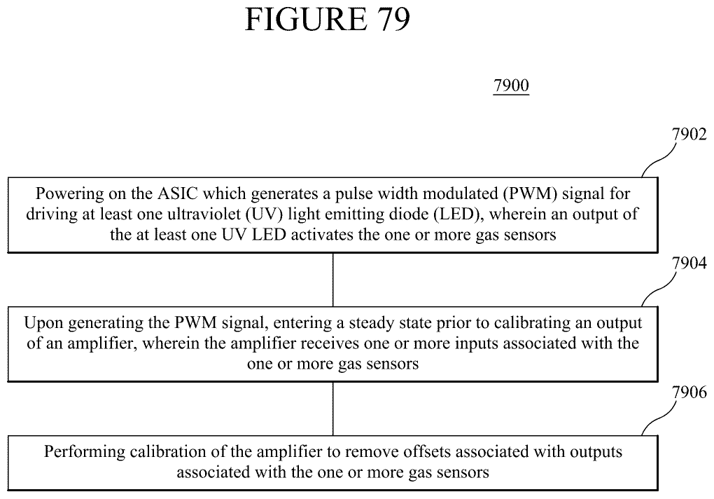

In addition to physical chips or ASICs, embodiments can also be expressed as methods, an example of which is provided in the flow chart of FIG. 79. Therein, a method 7900 for operating an Application Specific Integrated Circuit (ASIC) controlling one or more gas sensors includes the steps of powering on the ASIC 7902 which generates a pulse width modulated (PWM) signal for driving at least one ultraviolet (UV) light emitting diode (LED), wherein an output of the at least one UV LED activates the one or more gas sensors; upon generating the PWM signal, entering a steady state 7904 prior to calibrating an output of an amplifier, wherein the amplifier receives one or more inputs associated with the one or more gas sensors; and performing calibration 7906 of the amplifier to remove offsets associated with outputs associated with the one or more gas sensors.

### Reconfiguring Gas Sensing Devices to Detect New Gases

According to other embodiments, a gas sensing device can be reconfigured to enable it to detect one or more new (i.e., different) gases than it was previously configured to detect.

For example, there can be a gas sensor device which includes 5 gas sensors (S0, S1, S2, S6 and S8) with the device being currently calibrated (and/or configured) to detect and classify two gases, CH₄ and Cl₂. This gas sensor device can then be recalibrated (and/or configured) to detect and classify successively three gases, CH₄, Cl₂ and H₂S. The methods and systems which allow this change in detection from one or more gases to two or more gases are described below.

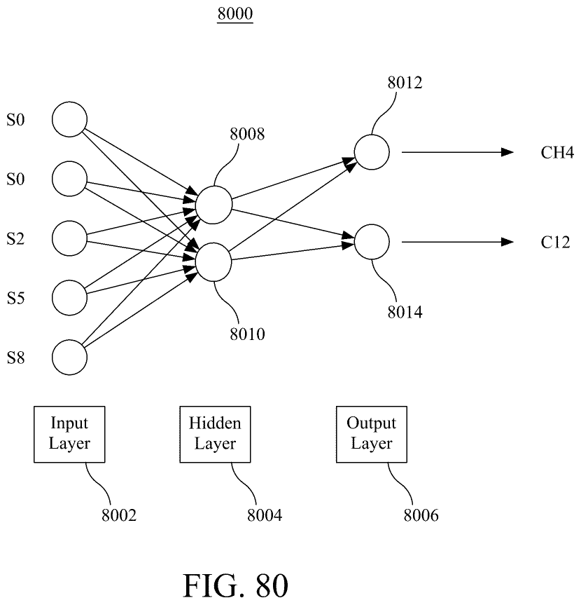

Initially, according to an embodiment, training occurs for the gas sensor to detect and classify Cl₂ and CH₄. FIG. 80 shows a neural network architecture 8000 which is used to detect and classify Cl₂ and CH₄. The neural network architecture 8000 includes five inputs representing the five gas sensors, S0, S1, S2, S6 and S8, as the input layer 8002, a hidden layer 8004 with two neurons 8008 and 8010 and an output layer 8006 which includes two neurons 8012 and 8014 for the two gases to be detected and classified. The first step with respect to the training is to collect the data for training the algorithm for the detection and identification of the two gases. To collect the data, the five sensor array is exposed to different gas concentrations of Cl₂ and CH₄ at different temperatures and relative humidity. Table XII represents the gas sensor's response when exposed to the different analytes. The data shown in Table XII is used to train the algorithm.

TABLE XII

| S0 | S1 | S2 | S6 | S8 | Gas Label |
|---|---|---|---|---|---|
| −0.14404 | 0.063258 | −0.0643 | 0.011754 | 0 | Cl2 |
| −0.03661 | 0.010371 | 0 | 0.000524 | 0 | Cl2 |
| −0.042 | 0.011965 | 0 | 0 | 0 | Cl2 |
| −0.07942 | 0.032559 | 0 | 0.006375 | 0 | Cl2 |
| −0.083 | 0.046961 | −0.00195 | 0.01059 | 0 | Cl2 |
| −0.2788 | 0.05075 | −0.00511 | 0 | 0 | Cl2 |
| −0.03747 | 0.036089 | 0 | 0.006375 | 0 | Cl2 |
| −0.09072 | 0.038797 | 0 | 0.007715 | 0 | Cl2 |
| 0 | 0 | 0 | 0 | 0.015807 | CH4 |
| 0 | 0 | 0 | 0 | 0.011807 | CH4 |
| 0 | 0 | 0 | 0 | 0.018807 | CH4 |
| 0 | 0 | 0 | 0 | 0.006674 | CH4 |

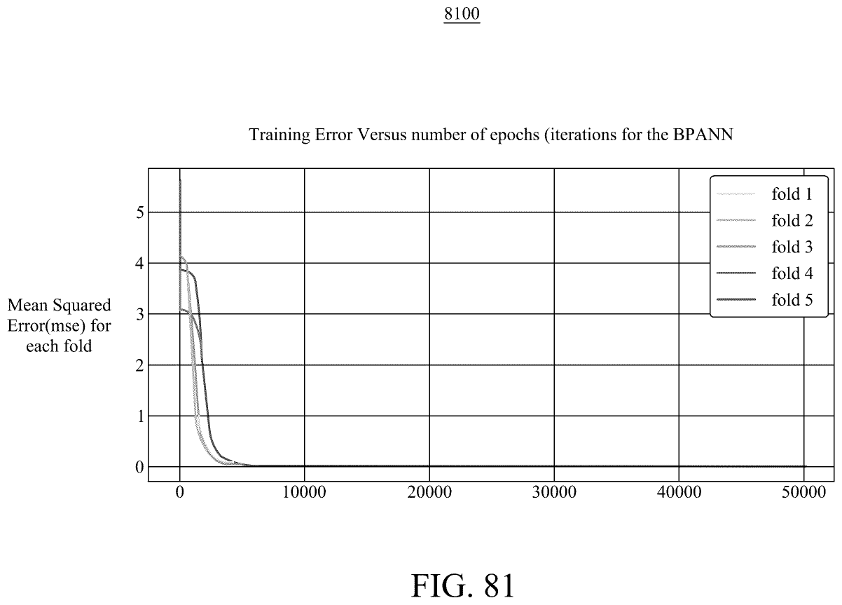

Further, regarding the training, FIG. 81 shows a graph 8100 which represents the variation of the mean square error of the cost function which describes the performance of the backpropagation artificial neural network (BPANN) algorithm as a function of the number of iterations. The mean square error in FIG. 81 converges to low values as a function of the number of iterations which can be interpreted as the minimization of the cost function where the weights of the neural network architecture remain unchanged (or at least substantially unchanged). Further each fold shown in FIG. 81 represents an equal portion of the data set used, e.g., in five folds each fold includes twenty percent of the data. Equation 13 includes the training coefficients obtained from the training and which are used as parameters in a function to detect and classify the two gases in real-time.

$${Θ}_{1} = ( \begin{matrix}3.9 & - 0.75 & 0.86 & 0.08 & 1.56 & - 1.42 \\ 32.23 & - 11.96 & 0.27 & - 0.76 & 12.39 & 0.4\end{matrix} ) \qquad ( 13 )$$

$${Θ}_{2} = ( \begin{matrix}1.66 & 23.59 & - 11. \\ - 1.43 & - 23.59 & 10.96\end{matrix} )$$

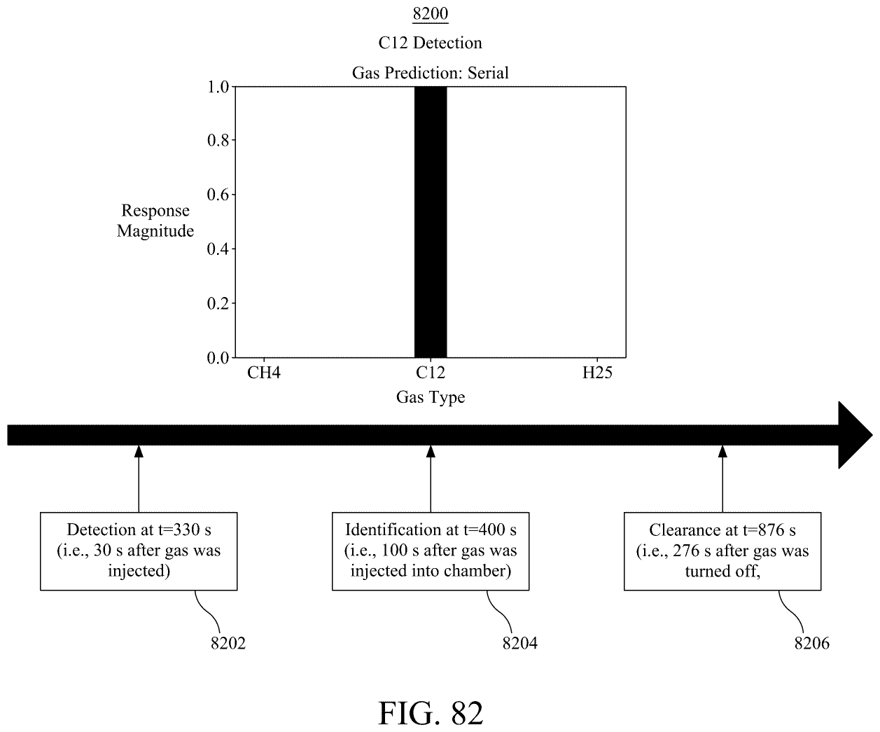

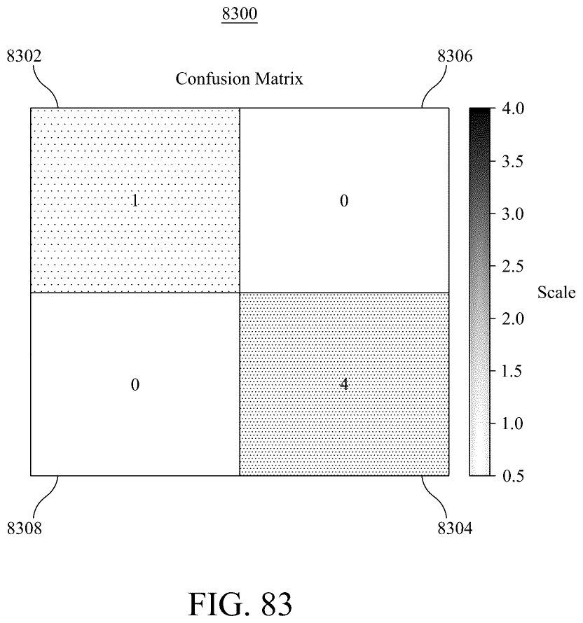

According to an embodiment, the results obtained for the detection of Cl₂ and the classification of Cl₂ with CH4 are shown in FIGS. 82 and 83, respectively. More specifically, FIG. 82 includes a sequence 8200 for detecting Cl₂ including the steps of detection 8202, identification 8204 and clearance 8206. With detection 8202 occurring 30 seconds after the gas was injected into the chamber, identification 8204 occurring 100 seconds after the gas was injected into the chamber and clearance 8206 occurring 276 seconds after the gas was turned off. With respect to FIG. 83, this shows a confusion matrix 8300 for Cl₂ classification with CH₄, wherein the confusion matrix 8300 provides a visualization of the performance of the algorithm with quadrant 8302 representing true positives, quadrant 8304 representing true negatives, quadrant 8306 representing false negatives and quadrant 8308 representing false positives.

Now that is has been described how the gas sensor was originally trained to detect two gases, how to train the same gas sensor for three gases, e.g., Cl₂, CH₄, and H₂S, i.e., including a new gas, will be described. According to an embodiment, the process for training three gases as compared to two gases for the gas sensor has numerous same and/or similar processes. As such, not every step will be repeated in this example.

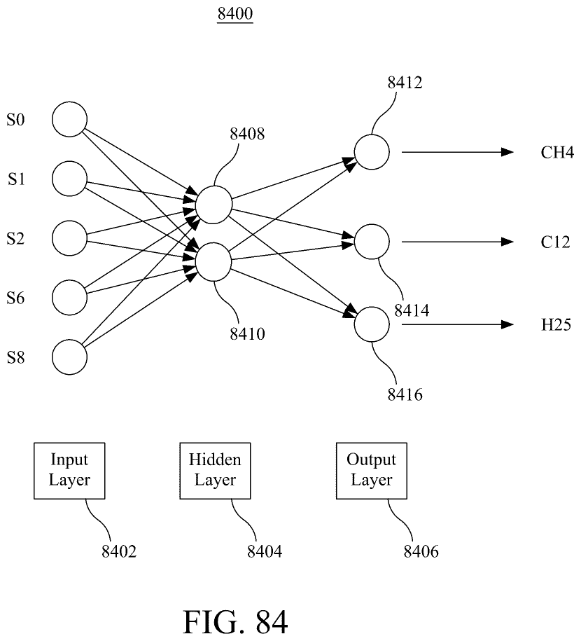

Initially, according to an embodiment, training occurs for the gas sensor to detect and classify Cl₂, CH₄ and H₂S. FIG. 84 shows a neural network architecture 8400 which is used to detect and classify the three gases. The neural network architecture 8400 includes five inputs representing the same five gas sensors, S0, S1, S2, S6 and S8, as the input layer 8402, a hidden layer 8404 with two neurons 8408 and 8410 and an output layer 8406 which includes three neurons 8412, 8414 and 8416 for the three gases to be detected and classified. The first step with respect to the training is to collect the data for training the algorithm for the detection and identification of the three gases. To collect the data, the five sensor array is exposed to different gas concentrations of Cl₂, CH₄ and H₂S at different temperatures and relative humidity. Table XIII represents the gas sensor's response when exposed to the different analytes. The data shown in Table XIII is used to train the algorithm.

TABLE XIII

| S0 | S1 | S2 | S6 | S8 | Gas Label |
|---|---|---|---|---|---|
| −0.14404 | 0.063258 | −0.0643 | 0.011754 | 0 | Cl2 |
| −0.03661 | 0.010371 | 0 | 0.000524 | 0 | Cl2 |
| −0.042 | 0.011965 | 0 | 0 | 0 | Cl2 |
| −0.07942 | 0.032559 | 0 | 0.006375 | 0 | Cl2 |
| −0.083 | 0.046961 | −0.00195 | 0.01059 | 0 | Cl2 |
| −0.2788 | 0.05075 | −0.00511 | 0 | 0 | Cl2 |
| −0.03747 | 0.036089 | 0 | 0.006375 | 0 | Cl2 |
| −0.09072 | 0.038797 | 0 | 0.007715 | 0 | Cl2 |
| −0.02632 | 0 | 0 | 0 | 0.000328 | H2S |
| −0.02777 | 0 | 0 | 0 | 0.000386 | H2S |
| −0.02507 | 0 | 0 | 0 | 0.000344 | H2S |
| −0.04576 | 0 | 0 | 0 | 0.000343 | H2S |
| −0.03775 | 0 | 0 | 0 | 0.000278 | H2S |
| −0.02728 | 0 | 0 | 0 | 0.000394 | H2S |
| 0 | 0 | 0 | 0 | 0.015807 | CH4 |
| 0 | 0 | 0 | 0 | 0.011807 | CH4 |
| 0 | 0 | 0 | 0 | 0.018807 | CH4 |
| 0 | 0 | 0 | 0 | 0.006674 | CH4 |

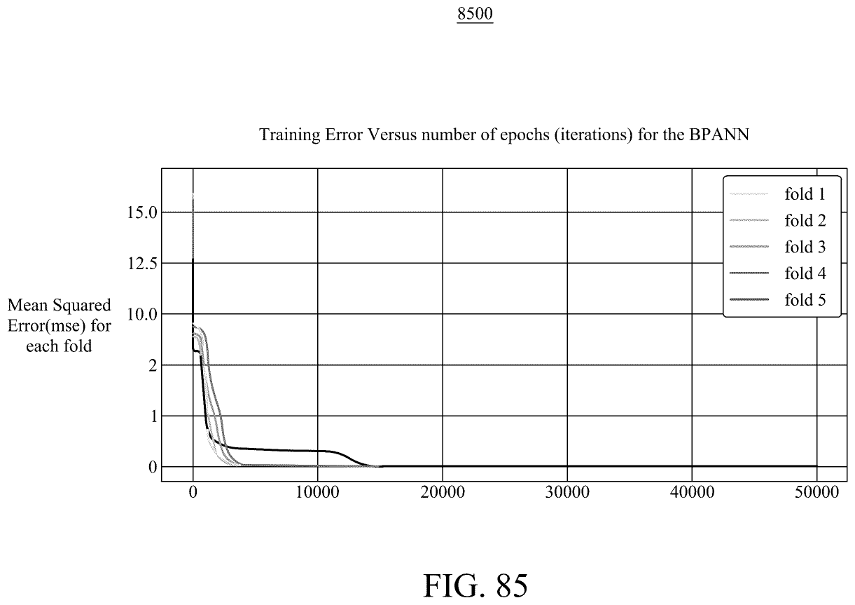

Further, regarding the training, FIG. 85 shows a graph 8500 which represents the variation of the mean square error of the cost function which describes the performance of the backpropagation artificial neural network (BPANN) algorithm as a function of the number of iterations. The mean square error in FIG. 85 converges to low values as a function of the number of iterations which can be interpreted as the minimization of the cost function where the weights of the neural network architecture remain unchanged (or substantially unchanged). Equation 14 includes the training coefficients obtained from the training and which are used as parameters in a function to detect and classify the three gases in real-time.

$${Θ}_{1} = ( \begin{matrix}- 28.25 & 16.71 & - 20. & 11.53 & - 9.33 & - 0.79 \\ - 8.02 & - 2.55 & 27.37 & - 12.96 & - 5. & 1.\end{matrix} ) \qquad ( 14 )$$

$${Θ}_{2} = ( \begin{matrix}26.25 & - 14.76 & - 7.25 \\ - 17.82 & - 20.37 & 13.56 \\ - 22.68 & 17.26 & 1.85\end{matrix} )$$

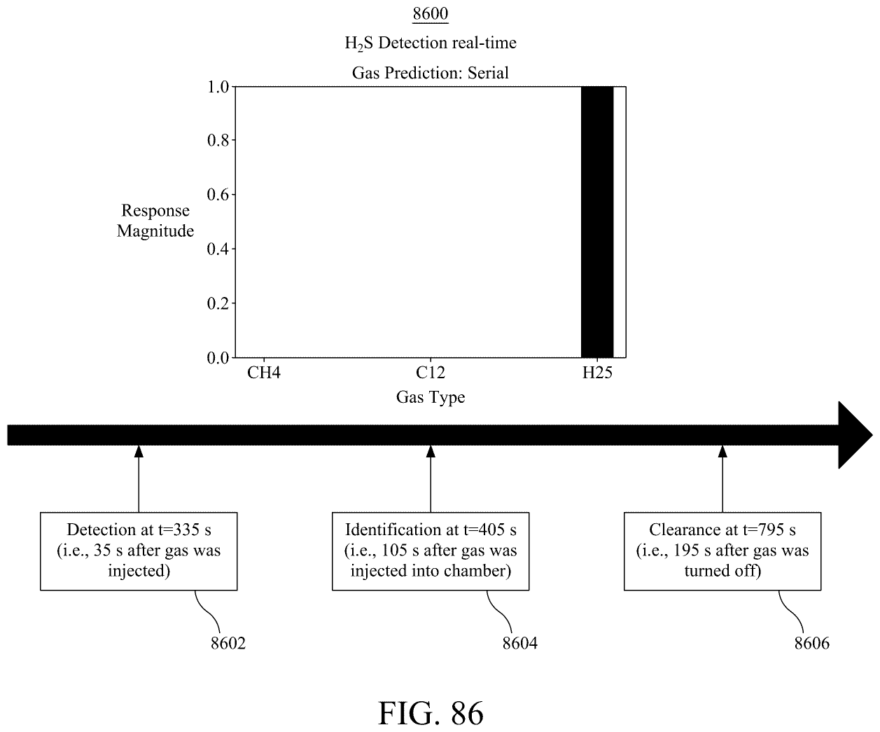

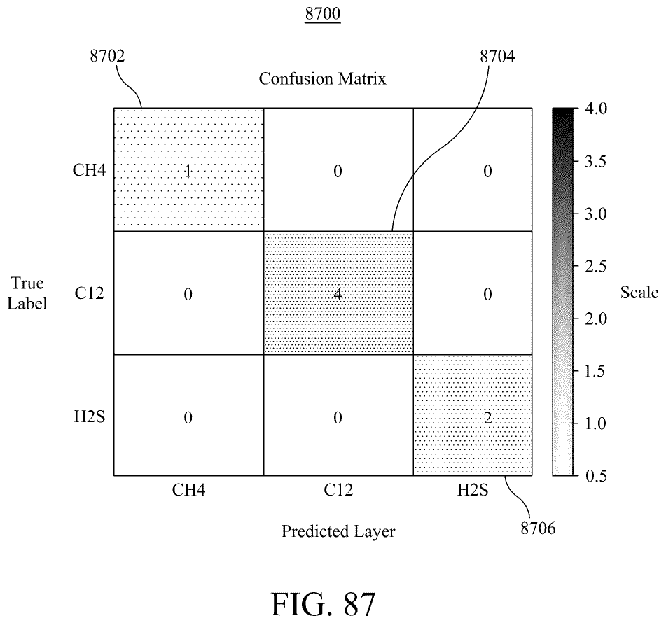

According to an embodiment, the results obtained for the detection of H₂S and the H₂S classification with CH₄ and Cl₂ are shown in FIGS. 86 and 87, respectively. More specifically, FIG. 86 includes a sequence 8600 for detecting H₂S including the steps of detection 8602, identification 8604 and clearance 8606. With detection 8602 occurring 35 seconds after the gas was injected into the chamber, identification 8604 occurring 105 seconds after the gas was injected into the chamber and clearance 8606 occurring 195 seconds after the gas was turned off. With respect to FIG. 87, this figure shows a confusion matrix 8700 for H₂S classification with CH₄ and Cl₂, wherein the confusion matrix 8700 provides a visualization of the performance of the algorithm by quadrant.

The three quadrants of primary interest 8702, 8704 and 8706 called the diagonal elements of the confusion matrix, represent the number of times for which the predicted gas is equal to the gas target. Specifically, for one exposure of CH₄ (8702)., the gas was identified correctly as CH₄. For four successive exposures of Cl₂ (8704), the algorithm accurately predicted the analyte as Cl₂. Finally, two exposures of H₂S (8706) were correctly identified as H₂S. In summary, there were no misclassification which is translated by the presence of the zeros in the off-diagonal elements of the confusion matrix.

The confusion matrix is just a visualization of the performance of the algorithm. It shows the number of the times the gas was correctly/incorrectly identified represented by the diagonal and off diagonal elements, respectively. Since it a visualization tool, its dimension always matches the number of elements to classify. Here, we are classifying three gases, so the dimension of the matrix is 3×3. If we were to classify two gases, the dimension would be 2×2.

According to an embodiment, it can be seen by comparing Equations (13) and (14) that the matrices associated with 02 are different. This difference, i.e., going from a 2×3 matrix to a 3×3 matrix, represents a difference in the weights (also known in the art as parameters) associated with reconfiguring the gas detection device from two gases to three gases in the information sent to the gas sensing device to be used in the gas sensing device's updating calibration.

The above-described examples associated with reconfiguring gas sensing devices to detect new gases show an example of going from detecting two gases to detecting three gases using the same five sensor array. It is to be understood that these quantities of gases and sensors are not intended to be limiting and that various combinations are potentially available depending on the gases to be detected and the makeup of the sensor array in the gas sensing device. For example, there can be a situation where it is desirable and possible to go from detecting a single gas to two gases, or from two gases to four gases, etc.

According to an embodiment, it is to be understood, that not all sensor functional nanoparticles can detect all gases, but that with enough different sensors in the device it is reasonable to expect that some reconfiguring can occur. Indeed the choice of functional nanoparticles can be made either (a) in a premeditated fashion to make sure you can add, e.g., H₂S later or (b) opportunistically to hope that you can reprogram the device for a desired new gas to be sensed.

According to an embodiment, the gas sensing system can be either a fixed gas sensing device or a wearable gas sensing device. The fixed gas sensing device can be a gas sensing device that remains in a fixed location to detect the chemicals in the air, e.g., placing the gas sensing device in an area where the output gases need to be monitored, or a wearable gas sensing device which a person can attach and wear on their clothing for monitoring as they move to various locations. Either gas sensing device can be, for example, the single package gas sensor described above. Alternatively, other designs can be used. Additionally, the type of gas sensing device used can influence what additional gases can be sensed as the type of sensor, i.e., the construction and materials selected for the sensor, can impact the types of gases for which the sensor array can be programmed to detect.

According to an embodiment, there are various ways the calibration process can be performed. For example, the gas sensor device can be returned to a location where the new calibration information can be uploaded to the device. Alternatively, with the appropriate communication setup, the data can be transmitted from a location to one or more gas sensing devices at their location of operation. For a third example, the data can be uploaded from a central device, which performed the initial training to generate the output weights/parameters for the recalibration, to a mobile device which can then be distributed to the locations where it is desirable to update the remote gas sensing devices and have the mobile device transmit the calibration information to the remote gas sensing devices to enable them to sense new/different gases.

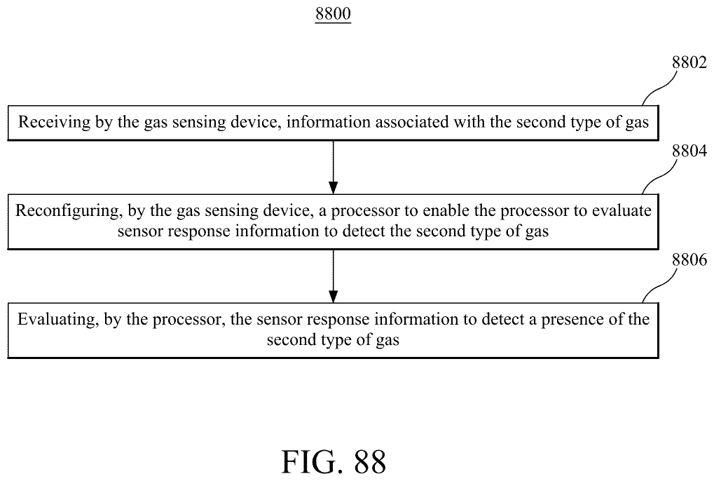

According to an embodiment, there is a method 8800 for reconfiguring a gas sensing device which is configured to detect a first type of gas to enable the gas sensing device to detect a second type of gas, as shown in FIG. 88. The method includes: in step 8802, receiving, by the gas sensing device, information associated with the second type of gas; in step 8804, reconfiguring, by the gas sensing device, a processor to enable the processor to evaluate sensor response information to detect the second type of gas; and in step 8806, evaluating, by the processor, the sensor response information to detect a presence of the second type of gas.

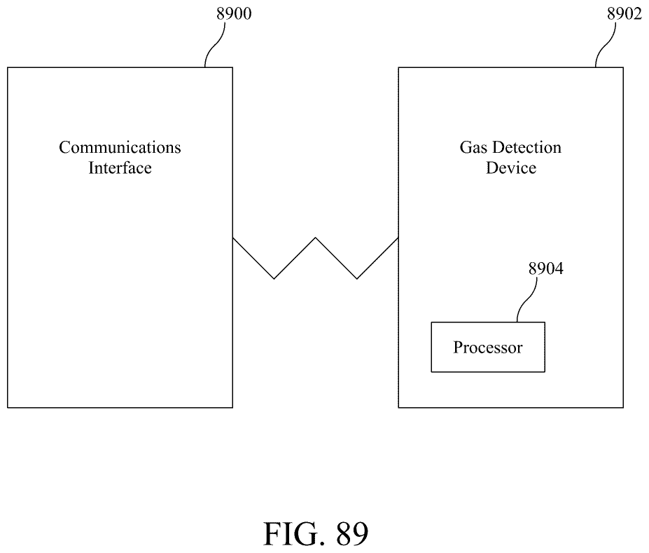

The foregoing method embodiment can include the use of various structures for performing the described steps. For example, as shown in FIG. 89, a system for reconfiguring a gas sensing device which is configured to detect a first type of gas to enable the gas sensing device to detect a second type of gas includes a communications interface 8900 configured to receive information associated with the second type of gas, e.g., the parameters described above. The gas detection device 8902 can include a processor 8904 configured to perform a reconfiguration to enable the gas detection device 8902 to evaluate sensor response information to detect a presence of the second type of gas. The processor 8904 can be included within the gas detection device 8902 or can also be remote therefrom. The communications interface 8900 and 8902 can communicate wirelessly (as shown) or via wireline.

### System for Detecting Conditions in an Environment Around a Device

According to other embodiments, there can be one or more devices configured to determine environmental conditions, e.g., atmospheric conditions, associated with a plurality of use cases that generate measurable characteristics. For example, the use cases can include, but are not limited to, wildfires, air pollution monitoring and air monitoring for a variety of gases which can then be implemented in a variety of places, e.g., smart cities, airports, railways, offices, forests, other outdoor environments, and areas where monitoring the environment could be useful for public safety. Initially embodiments are described with respect to a device, but further embodiments describe using one or more devices which can be connected to be a system for monitoring various environment conditions, collecting and interpreting associated environmental data, transmitting the data, evaluating the data and generating alerts based on rulesets, as well as transmitting and displaying such alerts on, for example, a user device. A user device can include such devices as computers with displays, mobile telephones, other smart devices and the like.

According to an embodiment, the device configured to determine environmental conditions is also known as a “ChemNode” sensing device. The ChemNode sensing device can be used as a part of a multimodal, distributed, early warning and threat detection platform for detecting a variety of threats and environmental conditions. For example, as described above, the ChemNode sensing device can be configured for fire detection, including localization and tracking. Further, environmental sensing capabilities can include, but are not limited to, NOₓ, O₃, SO₂, where x is an integer, as well as various particle sizes of Particulate Matter (PM) to include PM1.0, PM2.5, PM4.0 and PM10. System features, which can be varied and which are described in more detail below, can include solar power, a low power secure radio network, multimodal sensor data fusion, a rapid situational awareness with an Artificial Intelligence (AI) enabled cloud backend, integration with weather station data and Application Program Interfaces (APIs) associated with an alerting engine.

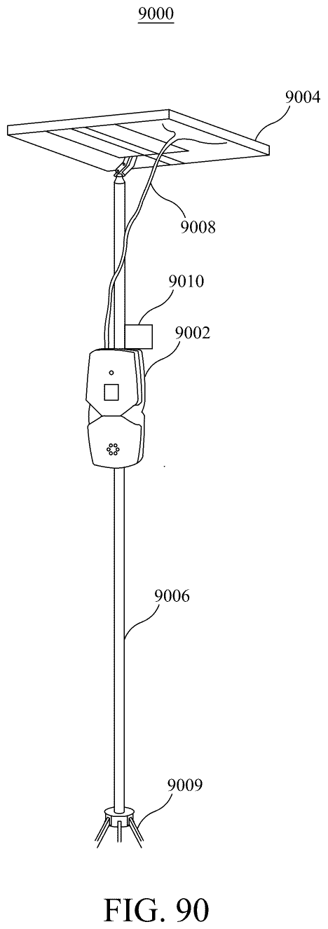

According to an embodiment, there is a device 9000, as shown in FIG. 90. Device 9000 (ChemNode sensing device) can include a sensor package 9002, a solar panel 9004 and a radio 9010. FIG. 90 also shows a stand 9006 to which the sensor package 9002, the solar panel 9004 and the radio 9010 are mounted. The stand or tripod 9006 can also be configured to stand on or otherwise be mounted to a surface, e.g., ground 9009 or a portion of a building. While shown in FIG. 90 as a single piece, the stand 9006 can be formed in a variety of manners to include multiple pieces to allow the stand 9006 to be deployed in a plurality of locations. For example the stand 9006 can be placed outdoors, or a shortened version of the stand 9006 can be mounted indoors. Further, while not shown separately in FIG. 90, a battery can be included as a part of the solar panel 9004, as a portion of the sensor package 9002 or as its own entity which can then also be attached to the stand 9006. Further a power cable 9008 can attach the solar panel 9004 to the sensor package 9002 or to one or more batteries.

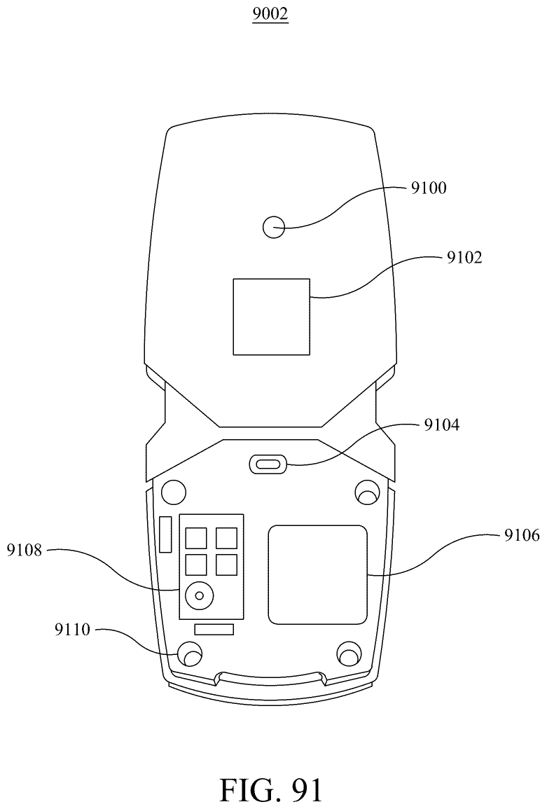

According to an embodiment, FIG. 91 shows the sensor package 9002 which is now described in more detail. According to an embodiment, the sensor package 9002 can include one or more of the following features: an infrared array sensor 9100, an e-paper screen 9102, a universal serial bus (USB) 9104, a particle monitor 9106, a gas sampling feature 9108 and a multiple gas sensor array 9110 which can be configured to detect one or more gases in the environment. The infrared array sensor 9100 can be an off the shelf infrared camera or other type of infrared detector with various sized viewing angles, e.g., a 60 degree viewing angle. The infrared array sensor 9100 can be used to identify point sources of heat that are within the unit line of sight. The e-paper screen 9102 can be an ultra-low power screen which can be used to interface with the sensor package 9002 on-site. The USB 9104 can be a USB-C as well as representing other desired types of connectivity functions. Further, the device 9000 and/or sensor package 9002 can be configured to include another camera which may be configured to capture images in a visible light spectrum. The e-paper screen 9102 can also allow for the use of QR codes. For example, a companion application running on a mobile phone, or other device, can be used to onboard the device in a system by using the QR code displayed on the screen, which can contain the serial number of the device and other details. Also, the e-paper screen 9102 can show working conditions such as battery power, and what communication network is currently connected, as well as describing failure codes for assistance with field diagnostics.

According to an embodiment, the particle monitor 9106 can be a high sensitivity particle monitor which is used to obtain particle quantities from the local environment. This information can support detection of wildfire smoke/smolder based on checking particle thresholds against an absolute or relative particle measure. The particle monitor 9106 can obtain data both rapidly and with a useful sensitivity while maintaining a good correlation to reference TSI mass concentration. The gas sampling feature 9108 and the gas sensor array 9110 can work together to capture and measure/detect various gas concentrations in the environment around the sensor package 9002. More specifically, an array of chemical sensors for gases like NOₓ, Os and SOₓ can be used to improve wildfire detection as compared to only attempting to detect a single gas type. According to an embodiment, the semiconductors used in the gas sensor array 9108, combined with desired programming (which can in some cases be modified at a future time to alter the gas combinations to be detected) determine what gases the gas sensor array is currently configured to detect. According to an embodiment, the combination of the various types of sensing devices and monitors are used in the gas sensor package 9002 to create a novel manner for detecting wildfires and/or other desired atmospheric/environment conditions.

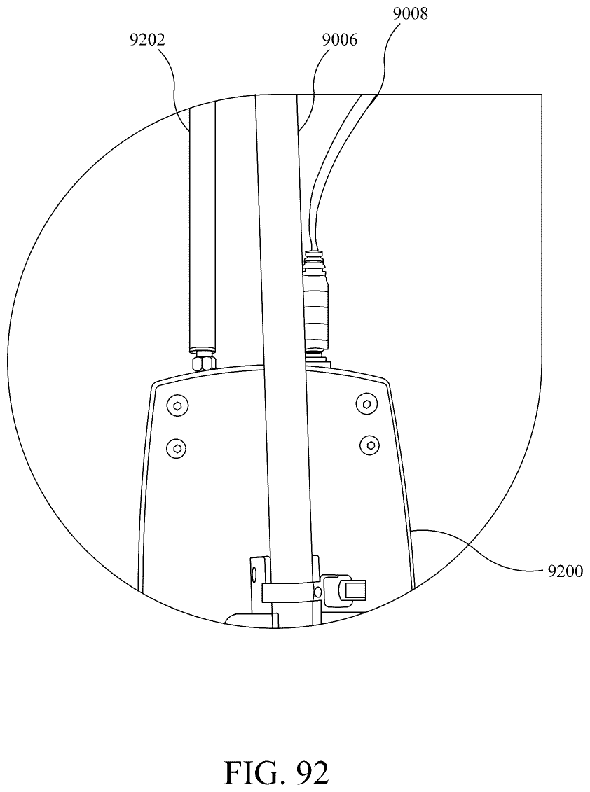

According to an embodiment, the device 9000 can have a radio as a portion of the device, with the radio 9200 shown in FIG. 92. FIG. 92 shows the radio 9200, an antenna 9202, the stand 9006 and the power cable 9008. The radio 9200 can either be a stand-alone radio function or it can be integrated with another portion of the device, such as the sensor package 9002. While using the term “radio” herein to describe the radio 9200, it is too be understood by those skilled in the art that the term “radio” is being broadly used herein to also include other devices which perform a similar function for transmitting and/or receiving information, e.g., optical transceivers, etc., For example, the radio 9200 can be a low-power radio with a gateway that can support multiple types of backhaul for use in areas without cellular connectivity, or the radio 9200 could be a device that uses cellular or satellite connectivity. The radio 9200 can be a device which is capable of operating using one or more radio access technologies (RATs). The radio 9200 can also be configured to operate in the Industrial, Scientific, and Medical (ISM) frequency bands, which are designated radio frequency bands as defined by the International Telecommunication Union (ITU) Radio Regulations. The most common everyday uses of the ISM bands are for low-power and short range telecommunications, such as WiFi, Bluetooth and the like for ease of communications to a gateway device.

According to an embodiment, regarding powering the Chemnode sensing device, in conditions when it is not feasible or desirable to have a traditional power system setup, the device 9000 can include a solar panel 9004 and a battery 9010. The solar panel 9010 can be a seventeen-Watt solar panel charger and integrated with a 30,000 milliampere hour (mAH) battery which can operate the device 9000 for up to seven days without sunlight. Alternatively, other combinations of types of solar panels 9004 and batteries 9010 could be used as desired based on the intended operating conditions.

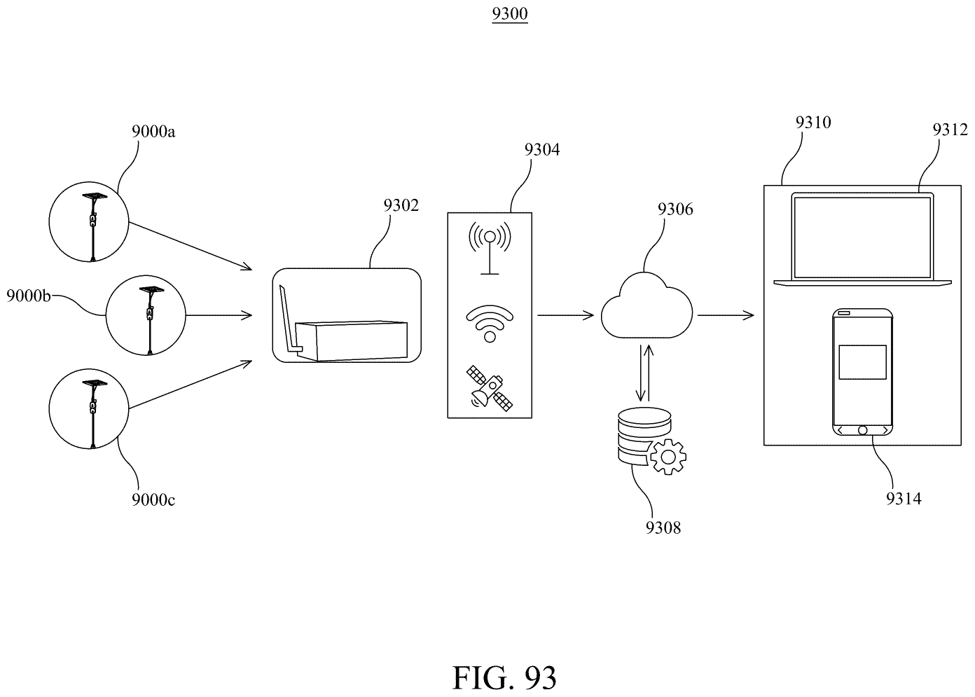

According to an embodiment, FIG. 93 shows a system 9300 which can support a process of detecting environmental conditions in an environment, e.g., wildfires/smoke, transmitting information, storing and processing information, transmitting the processed information and displaying the processed information in a variety of manners. Initially, a plurality of devices 9000a, 9000b, 9000c collect data associated with environmental conditions. This information is then transmitted to a gateway 9302 or other suitable device for further transmission, which tends to be of a longer range, to block 9304 which represents a longer distance type of communication device(s), e.g., Long Term Evolution (LTE) network, WiFi and a satellite backhaul. The information is then forwarded to a cloud backend 9306 which can include some form of artificial intelligence (AI). Depending on the whether the cloud backend 9306 includes the Al and depending on the capabilities of the Al, the received information can be processed to generate processed information which includes such things as map displays, alerts and transforming the sensed environmental information into a readable, usable format, e.g., percentages of one or more gases in the atmosphere per device 9000. Alternatively, the received information can be transmitted to a database 9308 (server) which can include and use a real-time analytics engine to perform the same functions as the functions previously described with the Al associated with the cloud backend 9306. The database 9308 and the cloud backend 9306 can be hosted on one or more servers. This processed information is then sent to one or more end user devices 9310 (which include a user interface) which can include, for example, a laptop 9312 and/or a user equipment 9314.

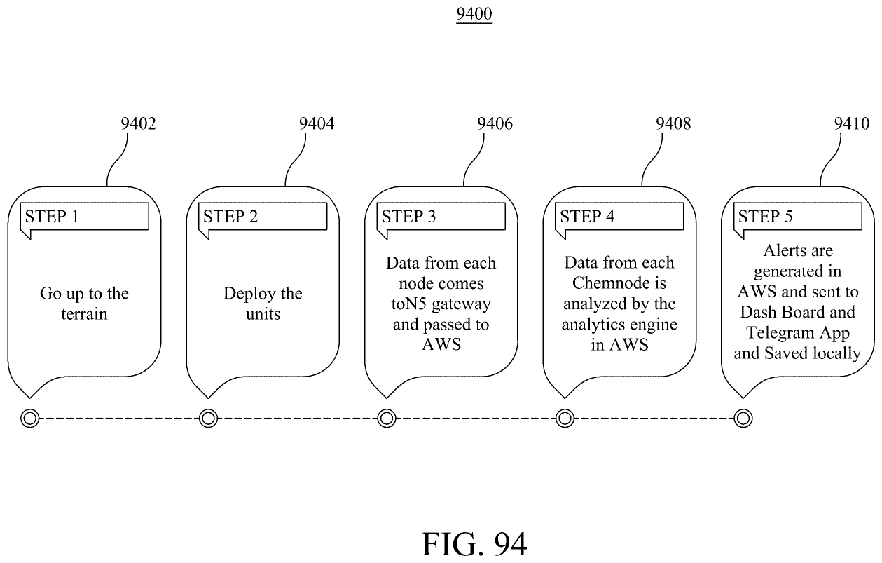

According to an embodiment, a test example implementing a set of devices 9000 will now be described with respect to FIGS. 94-97. Prior to discussing this example in detail with respect to FIGS. 94-97, a brief summary of the test is provided. Initially, a controlled burn test was performed using a system including eight ChemNode sensor systems (devices 9000) in communication with a cloud-based data processing capability and an alerting platform were deployed to detect and track fire ignitions during a controlled burn event. The devices 9000 were self-powered using solar panel(s) and included long range radios for allowing the devices 9000 to operate and communicate in remote environments. The system was able to detect fires in approximately 15 minutes in this test, however, wind speed, distance and burn progression speed could impact future results. The progress of the burn could be tracked over time in a map dashboard as fires were started and extinguished in different areas over time. Various data, both environmental and system associated, was gathered by the system and the devices 9000.

According to an embodiment, FIG. 94 shows the steps performed by a method 9400 in the controlled burn test when using the device(s) 9000 and the above described system 9300. In step 9402, the devices 9000 (i.e., ChemNode sensing devices) were delivered to the desired terrain. In step 9404 the devices were deployed. In step 9406, data/information from each device 9000 was transmitted to an N5 gateway (however, other commercially available gateways or the like could be used) and transmitted to the cloud-based data processing capability, e.g., an Amazon Web Service (AWS) function. In step 9408, the data from each device 9000 was analyzed by the analytics engine in the AWS function. In step 9410, alerts were generated in the AWS function and sent to a dashboard and telegram application on a user equipment (or equivalent device) for possible display and saved locally.

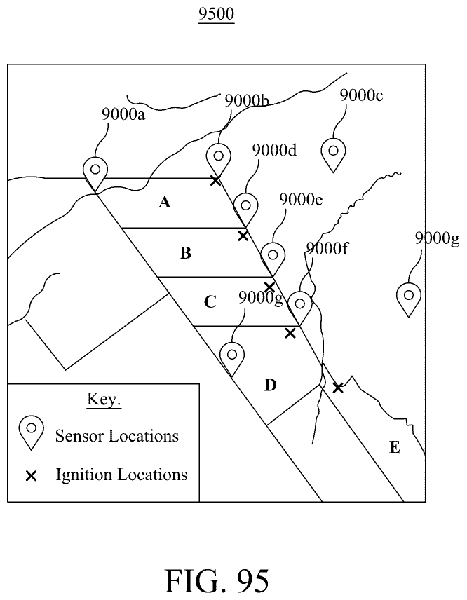

According to an embodiment, an example of an alert map 9500 is shown in FIG. 95. This alert map 9500 represents at least a portion of the controlled burn test area to include a plurality of burn zones A, B, C, D, E, device locations 9000a, 9000b, 9000c, 9000d, 9000e, 9000f and 9000g, and four ignition locations represented by an “x”. Alert map 9500 can also be used to depict a dashboard map representing the burn locations and device 9000a-g placement which can be displayed on the one or more end user devices 9310. It is to be understood that various formats for such dashboard displays can be created and displayed as desired.

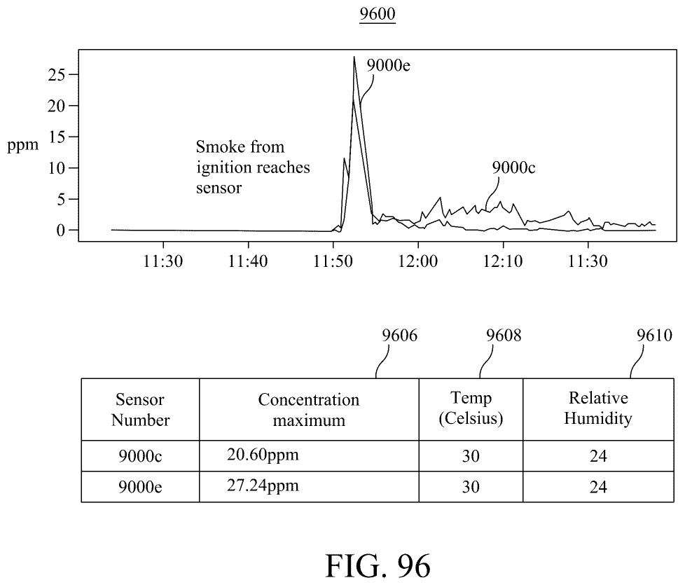

According to an embodiment, FIG. 96 shows an exemplary timing chart 9600 showing timing from ignition until smoke is detected as well as other potential information of interest. For example, timing chart 9600 includes graphical data 9602 associated with device 9000c and graphical data 9604 associated with device 9000e representing when smoke from one or more ignitions reaches and is detected by the respective sensor(s) on the device 9000c, 9000e. Also, FIG. 96 shows a concentration maximum in ppm 9606, a temperature 9608 and a relative humidity 9610 for each device.

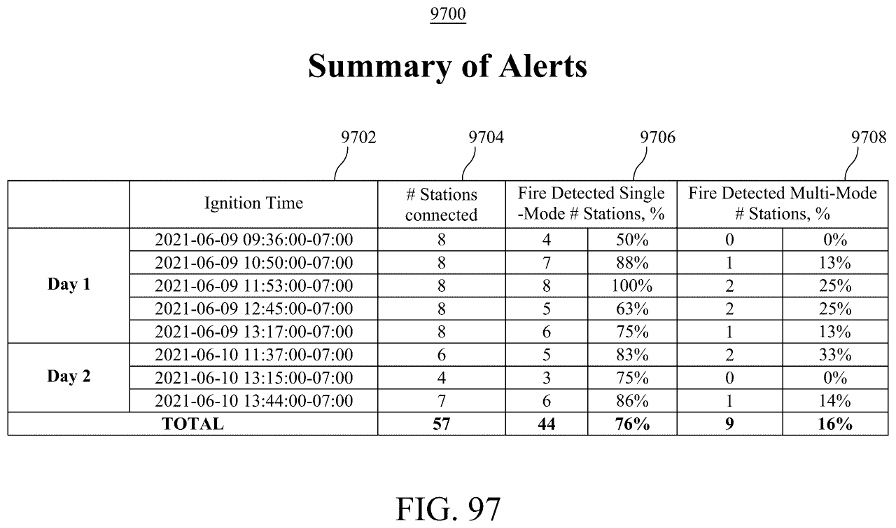

According to an embodiment, real-time data can be displayed on the end user devices 9310. For example, the data can be displayed in real-time for an area of interest, per device 9000a-9000g, or even in some cases by sensor type. Alternatively, nothing can be displayed until an alert is generated and transmitted by the system. The system can configure when such alerts should be generated or the alerts can be generated based on user inputs. Further, according to an embodiment, all or any subset of the generated data can be delivered in either a text or graphical format for use by the end users. This could be delivered in a summary format as shown in FIG. 97. In this example, the summary of alerts 9700 includes ignition time 9702, the number of stations connected 9704, the percentage of single devices 9000 detecting a fire 9706 and when a fire is detected from multiple devices 9000 by either a quantity of devices or the percentage of devices 9708. Other formats and data can be used in the summary of alerts as desired based on detection goals and/or end user desires. Other alert types can be tailored to the sensor modality. For example, a smoke alert from a PM sensor, a flame alert from an IR sensor. Additionally, additional sensors can provide local meteorological data such as wind speed, wind direction, temperature, humidity, etc., which can be useful for fighting a fire.

According to an embodiment, the device 9000 can include one or more functionalities to include gas sensor array(s), an IR camera and a particulate sensor module. Further the device 9000 can be connected to a cloud based real-time data analytics engine and a visualization dashboard as described above. The device 9000 can provide multimodal sensing which reduces false alarms and enables accurate detection in different climate and weather conditions. Unique software defined detection capability can allow for upgrading a chemical detection library with, for example, advancement in wildfire research. Further, the IR detection can occur both in day and night. The real time alert generation with a mapping capability can include Application Program Interfaces (APIs) for 3^{rd} party integration. Additionally, one or more of the gas sensor arrays can be made to be at least partially selective to ozone such that the sensor array element can be trained via an algorithm to detect and quantify an amount of ozone.

Embodiments described above have primarily focused on a single unit or a relatively small number of deployed devices 9000. It is too be understood that varying numbers can be deployed, e.g., 10, 50, 100, 500, 1000 or more, as desired. Hybrid deployment strategies can also reduce the total deployment cost, e.g., two variants of the device 9000 can be crated. For example, a higher priced variant could have all of the described functionalities, and a lower priced variant could have fewer functionalities. Varying combinations can be deployed to meet a variety of sensing needs over varying sizes and types of deployment areas.

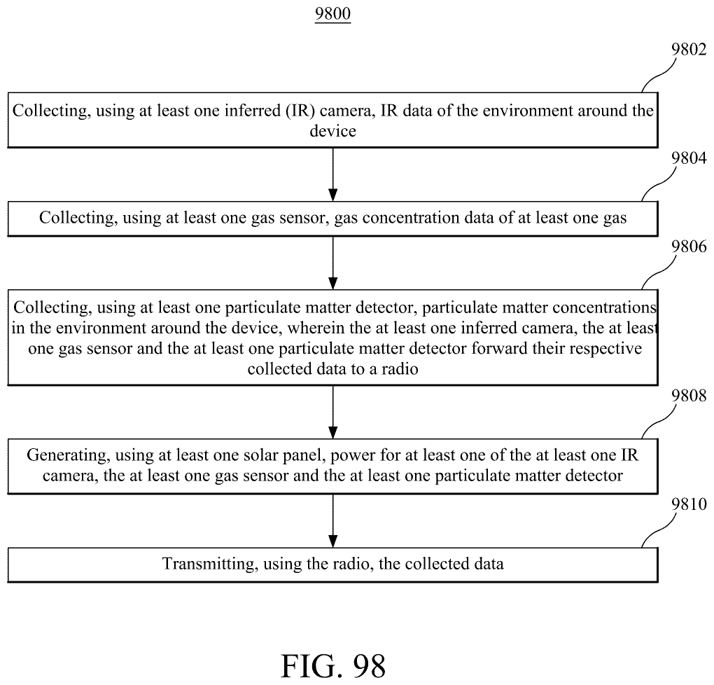

According to an embodiment, there is a method 9800 for collecting and transmitting information associated with conditions in an environment around a device as shown in FIG. 98. The method includes: at step 9802, collecting, using at least one infrared (IR) camera, IR data of the environment around the device; at step 9804, collecting, using at least one gas sensor, gas concentration data of at least one gas; at step 9806, collecting, using at least one particulate matter detector, particulate matter concentrations in the environment around the device, wherein the at least one infrared camera, the at least one gas sensor and the at least one particulate matter detector forward their respective collected data to a radio; at step 9808, generating, using at least one solar panel, power for at least one of the at least one IR camera, the at least one gas sensor and the at least one particulate matter detector; and at step 9810, transmitting, using the radio, the collected data.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

Although the features and elements of the present embodiments are described in the embodiments in particular combinations, each feature or element can be used alone without the other features and elements of the embodiments or in various combinations with or without other features and elements disclosed herein. The methods or flow charts provided in the present application may be implemented in a computer program, software, or firmware tangibly embodied in a computer-readable storage medium for execution by a general-purpose computer or a processor.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

1. A device configured to collect and transmit information associated with conditions in an environment around the device, the device comprising:
at least one infrared camera configured to collect IR data of the environment around the device;
at least one gas sensor configured to collect gas concentration data of at least one gas;
at least one particulate matter detector configured to collect particulate matter concentrations in the environment around the device, wherein the at least one infrared camera-, the at least one gas sensor and the at least one particulate matter detector forward their respective collected data to a radio;
at least one solar panel configured to generate power for at least one of the at least one IR camera, the at least one gas sensor and the at least one particulate matter detector; and
the radio configured to transmit the collected data.

2. The device of claim 1, wherein the at least one gas sensor is at least one of NOₓ, O₃ and SOₓ, wherein x is an integer.

3. The device of claim 1, wherein the device further comprises:
a tripod which is configured to have the at least one gas sensor, at least one solar panel and the radio attached to the tripod, wherein the tripod can be placed on or mounted to a surface.

4. The device of claim 1, further comprising:
a battery connected to the solar panel.

5. The device of claim 1, further comprising:
a second camera, wherein the second camera is configured to capture images in a visible light spectrum.

6. The device of claim 1, wherein the information collected from the environment around the device is information associated with at least one of air pollution, chemical gasses in air and wildfires.

7. A system for collecting data, processing the data, generating alerts and displaying the alerts in a real-time or near real-time basis associated with atmospheric content testing, the system comprising:
at least one infrared (IR) camera configured to collect IR data of the environment around the device;
at least one gas sensor configured to collect gas concentration data of at least one gas;
at least one particulate matter detector configured to collect particulate matter concentrations in the environment around the device, wherein at least one infrared camera, at least one gas sensor and at least one particulate matter detector forward their respective collected data to a radio, wherein the radio is configured to transmit the collected data to at least one server;
at least one solar panel configured to generate power for at least one IR camera, at least one gas sensor and at least one particulate matter detector;
at least one server configured to process the received data according to preconfigured instructions and to generate alerts based on a ruleset based on the processed data then to transmit the alerts; and
a user interface configured to receive and display the alerts.

8. At least one server configured to process received data according to preconfigured instructions and to generate alerts based on a ruleset using the processed data and then to transmit the alerts, wherein the received data is associated with at least one of air pollution, chemical gasses in air and wildfires.

9. At least one server of claim 8, wherein the preconfigured instructions and the ruleset can be updated to reflect a change in the generated alerts.

10. At least one server of claim 9, wherein the preconfigured instructions and the ruleset are configured to generate alerts based on a quantity of SOₓ, wherein x is an integer.

11. At least one server of claim 10, wherein the preconfigured instructions and the ruleset are updated to generate alerts based on a quantity of NOₓ, wherein x is an integer.

12. An integrated sensor suite configured to collect and transmit information associated with atmospheric conditions, the integrated sensor suite comprising:
a plurality of sensors configured to capture the information associated with the atmospheric conditions, wherein the atmospheric conditions include air temperature, at least one gas concentration and a particulate matter count.

13. The integrated sensor suite of claim 12, wherein the gas concentration is at least one of NOₓ, Os and SOₓ, wherein x is an integer.

14. The integrated sensor suite of claim 12, wherein the plurality of sensors is attached to a tripod.

15. The integrated sensor suite of claim 12, further comprising:
at least one solar panel configured to generate power for the plurality of sensors.

16. The integrated sensor suite of claim 12, further comprising:
a camera, wherein the camera is configured to capture images in a visible light spectrum.

17. The integrated sensor suite of claim 12 further comprising:
a communications interface configured to transmit the information associated with the atmospheric conditions to at least one server configured to process the received information associated with the atmospheric conditions according to preconfigured instructions and to generate alerts based on a ruleset using the processed data and then to transmit the alerts to a user device.

18. A method for collecting and transmitting information associated with conditions in an environment around a device, the method comprising:
collecting, using at least one infrared camera, IR data of the environment around the device;
collecting, using at least one gas sensor, gas concentration data of at least one gas;
collecting, using at least one particulate matter detector, particulate matter concentrations in the environment around the device, wherein the at least one infrared camera, at least one gas sensor and at least one particulate matter detector forward their respective collected data to a radio;
generating, using at least one solar panel, power for at least one IR camera, at least one gas sensor and at least one particulate matter detector; and
transmitting, using the radio, the collected data.

19. The method of claim 18, wherein at least one gas sensor is at least one of NOₓ, O₃ and SOₓ, wherein x is an integer.

20. The method of claim 18, wherein the device is attached to a tripod.

21. The method of claim 18, further comprising:
connecting a battery to the solar panel.

22. The method of claim 18, further comprising:
capturing, using a second camera, images in a visible light spectrum.

23. The method of claim 18, wherein the information collected from the environment around the device is information associated with at least one of air pollution, chemical gasses in air and wildfires.
